(19) United States
(12) Patent Application Publication
(10) Pub. No.: US 2026/0337195 A1
(43) Pub. Date: Oct. 29, 2026

(54) SOLID FORMS OF AN ALPHA4-BETA7 INTEGRIN ANTAGONIST AND COMPOUNDS THEREOF

(71) Applicant: Gilead Sciences, Inc., Foster City, CA (US)

(72) Inventors: Kassibla E. Dempah, San Francisco, CA (US); Jesse M. Jacobsen, Seattle, WA (US); Yasamin Moazami, Seattle, WA (US); Jeffrey D. Ng, South San Francisco, CA (US); Bing Shi, Redwood City, CA (US); Todd A. Wenderski, Hayward, CA (US)

(21) Appl. No.: 19/550,567

(22) Filed: Feb. 26, 2026

Related U.S. Application Data
(60) Provisional application No. 63/764,870, filed on Feb. 28, 2025.

(51) Int. Cl. C07D 471/04; A61K 31/519; A61K 31/5377

(52) U.S. Cl. CPC C07D 471/04; A61K 31/519; A61K 31/5377

(57) ABSTRACT

Disclosed herein are solid forms and compounds which act as inhibitors or modulators of α4β7 integrins. The disclosed also relates to pharmaceutical compositions containing one or more of the solid forms or compounds disclosed herein, as well as methods for using the solid forms or compounds in treatment of conditions mediated by α4β7 integrin. The disclosure also relates to methods for obtaining such solid forms or compounds

### CROSS-REFERENCE TO RELATED APPLICATIONS

This application claims the benefit under 35 U.S.C. § 119(e) of U.S. Provisional Application No. 63/764,870, filed on Feb. 28, 2025, which is hereby incorporated herein by reference in its entirety for all purposes

### FIELD

The present disclosure relates to solid forms and compounds of alpha4-beta7(α4β7) integrin antagonists, and pharmaceutical formulations and therapeutic uses thereof.

### BACKGROUND

Integrins are heterodimeric cell surface proteins involved in numerous cellular processes including cell-cell and cell-extracellular matrix interactions. α4β7 integrin is expressed on the surface of lymphocytes and recognizes the extracellular ligand mucosal addressing cell adhesion molecule-1 (MAdCAM-1) and governs lymphocyte trafficking to and retention in gut tissues. Interaction of α4β7 integrin with MAdCAM-1 has been implicated as an important contributor to the chronic intestinal inflammation that is a hallmark of ulcerative colitis (UC) and Crohn's disease (CD).

Compounds that bind to α4β7 integrin can act as modulators of α4β7 integrin. α4β7 integrin inhibitors can be useful for treating and/or prophylaxis of UC and CD through binding and inhibiting lymphocyte trafficking and retention in gut tissues. One compound useful for inhibiting α4β7 integrin is a compound of Formula I:

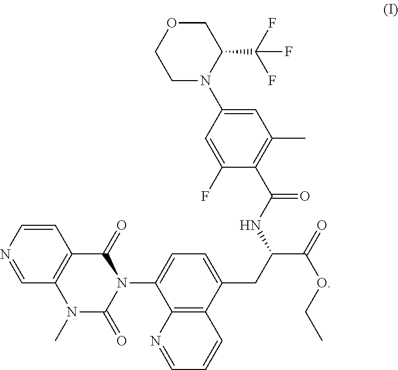

Although there are known inhibitors of α4β7 integrins, what is desired in the art are physically stable forms, such as a compound of Formula I, or pharmaceutically acceptable salts thereof, with desired properties such as good physical and chemical stability, good aqueous solubility, and good bioavailability. For example, pharmaceutical compositions are desired that address challenges of stability, variable pharmacodynamics responses, drug-drug interactions, pH effects, food effects, and/or bioavailability.

Accordingly, there is a need for stable forms of a compound of Formula I with suitable chemical and physical stability for the formulation, therapeutic use, manufacturing, and storage of the compound.

A solid form may have properties such as bioavailability, stability, purity, and/or manufacturability at certain conditions that may be suitable for medical or pharmaceutical uses.

### SUMMARY

In some embodiments, the present disclosure is directed to solid forms of Formula I:

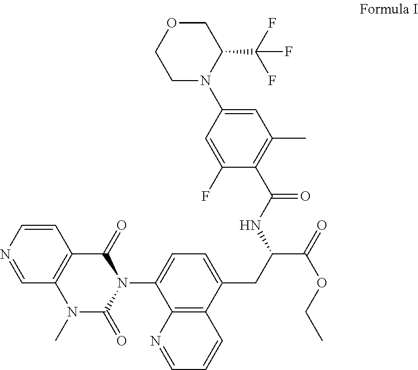

and pharmaceutically acceptable salts, solvates and hydrates thereof.

These forms are useful, for example, for treating human patients suffering from an inflammatory disease or condition, such as inflammatory bowel disease (IBD), such as ulcerative colitis (UC) or Crohn's disease (CD), metabolic dysfunction-associate steatohepatitis (MASH), primary sclerosing cholangitis (PSC), graph-versus-host-disease (GVHD), alcoholic hepatitis, alcoholic steatohepatitis (ASH), autoimmune hepatitis, alcoholic hepatitis, and systemic lupus erythematosus (SLE). The solid forms of the present disclosure can be useful for preparing a medicament for treating an inflammatory disease. The solid forms of the present disclosure can be used to inhibit α4β7 integrin.

In some embodiments, the present disclosure is directed to a Formula I freebase Form I.

In some embodiments, the present disclosure is directed to a Formula I freebase acetophenone solvate.

In some embodiments, the present disclosure is directed to a Formula I freebase anisole solvate.

In some embodiments, the present disclosure is directed to a solid form of a compound of the following formula:

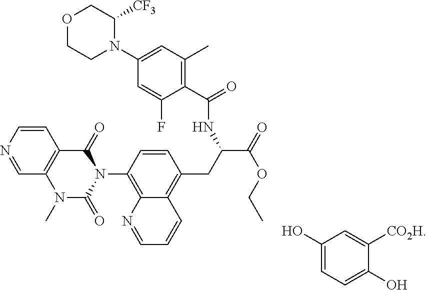

In some embodiments, the present disclosure is directed to a Formula I gentisate Form I.

In some embodiments, the present disclosure is directed to a solid form of a compound of the following formula:

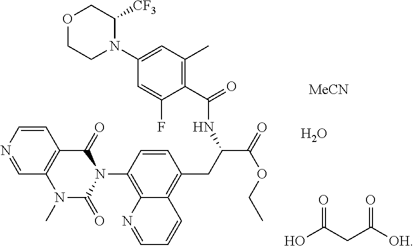

In some embodiments, the present disclosure is directed to a Formula I mono-malonate monohydrate acetonitrile solvate.

In some embodiments, the present disclosure is directed to solid form of a compound of the following formula:

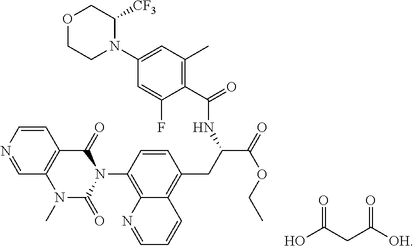

In some embodiments, the present disclosure is directed to a Formula I mono-malonate Form I.

In some embodiments, the present disclosure is directed to a Formula I mono-malonate Form II.

In some embodiments, the present disclosure is directed to solid forms of a compound of the following formula:

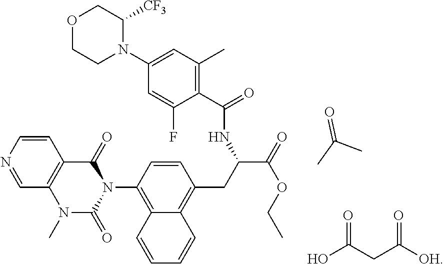

In some embodiments, the present disclosure is directed to a Formula I mono-malonate acetone solvate.

In some embodiments, the present disclosure is directed to solid forms of a compound of the following formula:

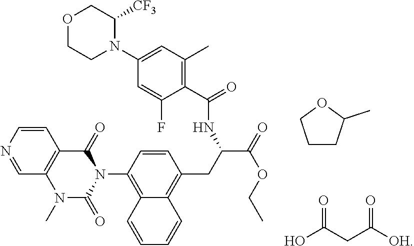

In some embodiments, the present disclosure is directed to a Formula I mono-malonate 2-methyltetrahydrofuran solvate.

In some embodiments, the present disclosure is directed to a solid form of a compound of the following formula:

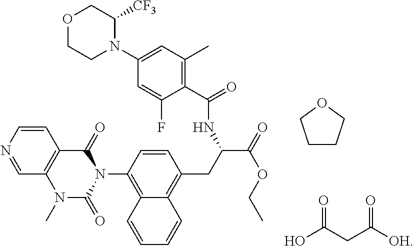

In some embodiments, the present disclosure is directed to a Formula I mono-malonate tetrahydrofuran solvate.

In some embodiments, the present disclosure is directed to a solid form of a compound of the following formula:

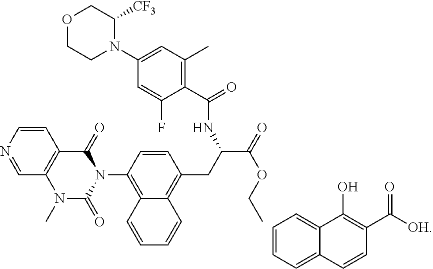

In some embodiments, the present disclosure is directed to a Formula I xinafoate Form I.

In some embodiments, the present disclosure is directed to a Formula I xinafoate Form II.

In some embodiments, the present disclosure is directed to a solid form of a compound of the following formula:

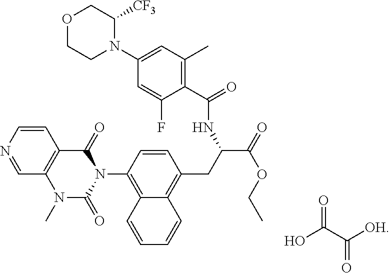

In some embodiments, the present disclosure is directed to a Formula I oxalate.

In some embodiments, the present disclosure is directed to a compound of the following formula:

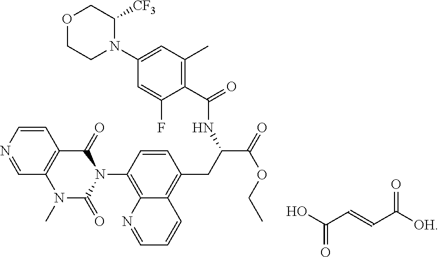

In some embodiments, the present disclosure is directed to a Formula I fumarate Form I.

In some embodiments, the present disclosure is directed to a Formula I fumarate Form II.

In some embodiments, the present disclosure is directed to a Formula I fumarate Form III.

In some embodiments, the present disclosure is directed to a compound of the following formula:

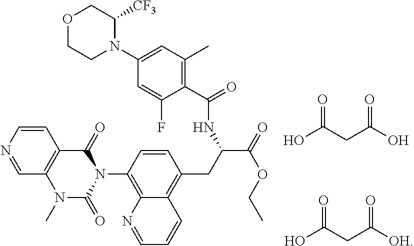

In some embodiments, the present disclosure is directed to a Formula I bis-malonate.

In some embodiments, the present disclosure is directed to a compound of the following formula:

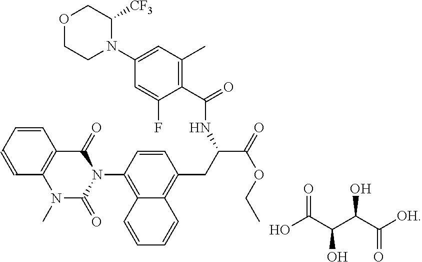

In some embodiments, the present disclosure is directed to a Formula I L-tartrate.

In some embodiments, the present disclosure is directed to solid forms of a compound of the following formula:

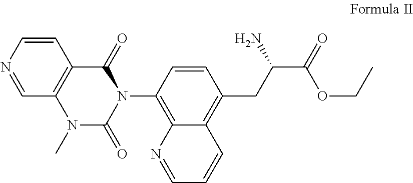

and a pharmaceutically acceptable salt thereof.

In some embodiments, the present disclosure is directed to a Formula II freebase Form I.

In some embodiments, the present disclosure is directed to a Formula II freebase Form II.

In some embodiments, the present disclosure is directed to a Formula II freebase Form III.

In some embodiments, the present disclosure is directed to a Formula II freebase Form IV.

In some embodiments, the present disclosure is directed to a Formula II freebase Form V.

In still other embodiments, the present disclosure is directed to compounds useful in modulating an α4β7 integrin. In some embodiments, the present disclosure is directed to a compound, or pharmaceutically acceptable salt, which is;

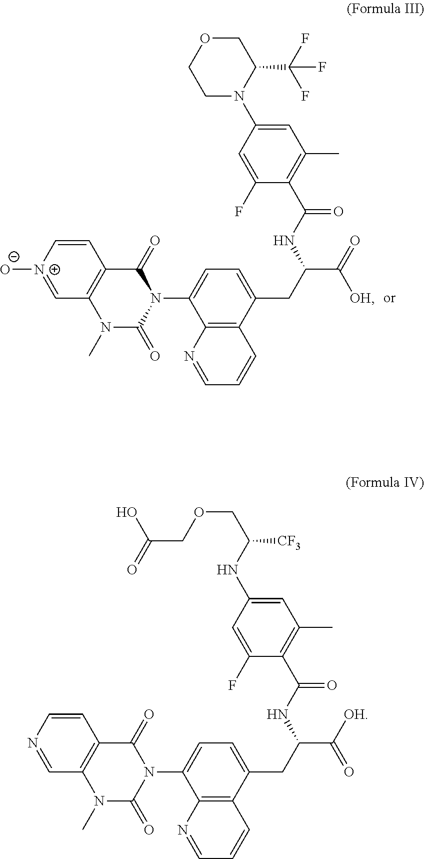

In some embodiments, the present disclosure is directed to a pharmaceutical composition comprising a compound which is

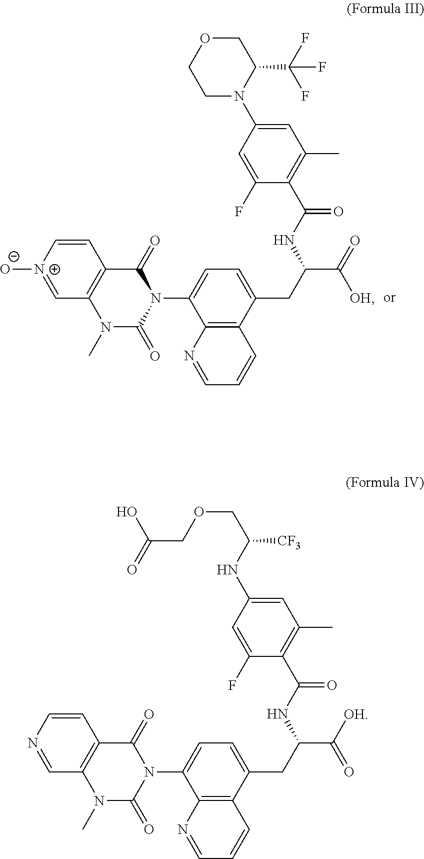

or a pharmaceutically acceptable salt thereof, and a pharmaceutically acceptable excipient.

In some embodiments, the present disclosure is directed to a compound of

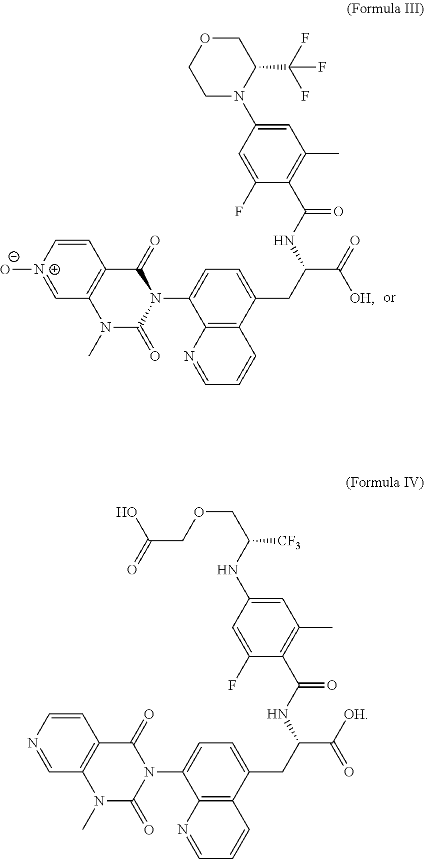

or a pharmaceutically acceptable salt thereof, for use in medical therapy.

In some embodiments, the present disclosure is directed to a method for treating a disease or condition associated with α4β7 integrin is disclosed, wherein the method comprises administrating to a subject an effective amount of a compound, wherein the compound is

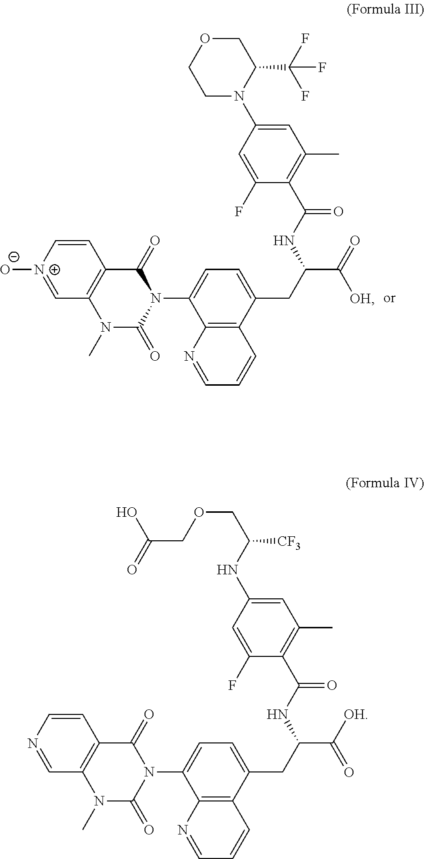

or a pharmaceutically acceptable salt thereof.

In some embodiments, the present disclosure is directed to a compound of the following formula:

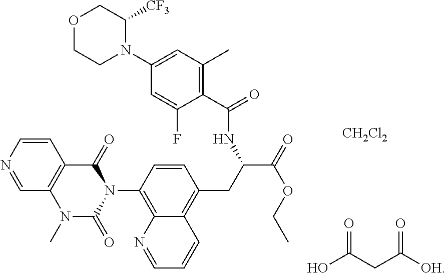

In some embodiments, the present disclosure is directed to a Formula I mono-malonate dichloromethane solvate.

In some embodiments, the present disclosure is directed to a compound of the following formula:

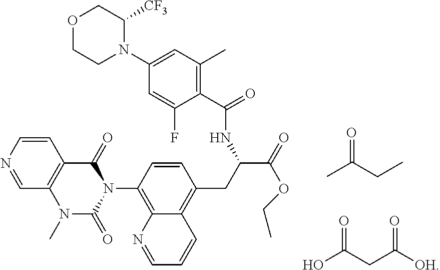

In some embodiments, the present disclosure is directed to a Formula I mono-malonate methyl ethyl ketone solvate.

### BRIEF DESCRIPTION OF THE FIGURES

FIG. 1 shows an XRPD pattern of Formula I freebase Form I.

FIG. 2 shows a DSC thermogram of Formula I freebase Form I.

FIG. 3 shows a TGA thermogram of Formula I freebase Form I.

FIG. 4 shows an XRPD pattern of Formula I freebase acetophenone solvate.

FIG. 5 shows a DSC thermogram of Formula I freebase acetophenone solvate.

FIG. 6 shows a TGA thermogram of Formula I freebase acetophenone solvate.

FIG. 7 shows a XRPD pattern of Formula I freebase anisole solvate.

FIG. 8 shows a DSC thermogram of Formula I freebase anisole solvate.

FIG. 9 shows a TGA thermogram of Formula I freebase anisole solvate.

FIG. 10 shows an XRPD pattern of Formula I gentisate Form I.

FIG. 11 shows a DSC thermogram of Formula I gentisate Form I.

FIG. 12 shows a TGA thermogram of Formula I gentisate Form I.

FIG. 13 shows an XRPD pattern of Formula I mono-malonate monohydrate acetonitrile solvate.

FIG. 14 shows a DSC thermogram of Formula I mono-malonate monohydrate acetonitrile solvate.

FIG. 15 shows a TGA thermogram of Formula I mono-malonate monohydrate acetonitrile solvate.

FIG. 16 shows an XRPD pattern of Formula I mono-malonate Form I.

FIG. 17 shows a DSC thermogram of Formula I mono-malonate Form I.

FIG. 18 shows a TGA thermogram of Formula I mono-malonate Form I.

FIG. 19 shows an XRPD pattern of Formula I mono-malonate Form II.

FIG. 20 shows a DSC thermogram of Formula I mono-malonate Form II.

FIG. 21 shows a TGA thermogram of Formula I mono-malonate Form II.

FIG. 22 shows an XRPD pattern of Formula I mono-malonate acetone solvate.

FIG. 23 shows an XRPD pattern of Formula I mono-malonate 2-methyltetrahydrofuran solvate.

FIG. 24 shows an XRPD pattern of Formula I mono-malonate tetrahydrofuran solvate.

FIG. 25 shows an XRPD pattern of Formula I xinafoate Form I.

FIG. 26 shows an XRPD pattern of Formula I xinafoate Form II.

FIG. 27 shows a DSC thermogram of Formula I xinafoate Form II.

FIG. 28 shows a TGA thermogram of Formula I xinafoate Form II.

FIG. 29 shows an XRPD pattern of Formula I oxalate.

FIG. 30 shows a DSC thermogram of Formula I oxalate.

FIG. 31 shows a TGA thermogram of Formula I oxalate.

FIG. 32 shows an XRPD pattern of Formula I fumarate Form I.

FIG. 33 shows a DSC thermogram of Formula I fumarate Form I.

FIG. 34 shows a TGA thermogram of Formula I fumarate Form I.

FIG. 35 shows an XRPD pattern of Formula I fumarate Form II.

FIG. 36 shows a DSC thermogram of Formula I fumarate Form II.

FIG. 37 shows a TGA thermogram of Formula I fumarate Form II.

FIG. 38 shows an XRPD pattern of Formula I fumarate Form III.

FIG. 39 shows an XRPD pattern of Formula I bis-malonate Form I.

FIG. 40 shows a DSC thermogram of Formula I bis-malonate Form I.

FIG. 41 shows a TGA thermogram of Formula I bis-malonate Form I.

FIG. 42 shows an XRPD pattern of Formula I L-tartrate.

FIG. 43 shows a DSC thermogram of Formula I L-tartrate.

FIG. 44 shows a TGA thermogram of Formula I L-tartrate.

FIG. 45 shows an XRPD pattern of Formula II Form I.

FIG. 46 shows an XRPD pattern of Formula II Form II.

FIG. 47 shows a DSC thermogram of Formula II Form II.

FIG. 48 shows a TGA thermogram of Formula II Form II.

FIG. 49 shows an XRPD pattern of Formula II Form III.

FIG. 50 shows an XRPD pattern of Formula II Form IV.

FIG. 51 shows an XRPD pattern of Formula II Form V.

FIG. 52 shows an XRPD pattern of Formula I mono-malonate dichloromethane solvate.

FIG. 53 shows an XRPD pattern of Formula I mono-malonate methyl ethyl ketone solvate.

FIG. 54 shows the binding of Formula III and Formula IV to the α4β7 integrin receptor in human CD 4 T_{memory} cells.

FIG. 55 shows the binding of Formula III and Formula IV to the α4β7 integrin receptor in human CD 8 T_{memory} cells.

FIG. 56 shows a DSC thermogram of Formula II freebase Form IV.

FIG. 57 shows an XRPD pattern of Formula II freebase Form VI.

FIG. 58 shows a DSC thermogram of Formula II freebase Form VI.

FIG. 59 shows an XRPD pattern of Formula II freebase Form VII.

FIG. 60 shows an XRPD pattern of Formula II freebase Form VIII.

FIG. 61 shows an XRPD pattern of Formula II freebase Form IX.

FIG. 62 shows an XRPD pattern of Formula II freebase Form X.

### DETAILED DESCRIPTION

In the following description, certain specific details are set forth in order to provide a thorough understanding of various embodiments of the disclosure. However, one skilled in the art will understand that the disclosure may be practiced without these details. The description below of several embodiments is made with the understanding that the present disclosure is to be considered as an exemplification of the claimed subject matter and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### DEFINITIONS

Unless the context requires otherwise, throughout the present specification and claims, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments that reference throughout this specification to “a compound” include the crystalline, salt, co-crystal, freebase, hydrate, solvate, and/or amorphous forms of the formulas and/or compounds disclosed herein. Thus, the appearance or the phrase “a compound of Formula I” can include Formula I freebase Form I; Formula I freebase acetophenone solvate; Formula I freebase anisole solvate; Formula I gentisate Form I; Formula I mono-malonate monohydrate acetonitrile solvate; Formula I mono-malonate Form I; Formula I mono-malonate From II; Formula I mono-malonate acetone solvate; Formula I mono-malonate 2-methyltetrahydrofuran solvate; Formula I mono-malonate tetrahydrofuran solvate; Formula I xinafoate Form I; Formula I xinafoate Form II; Formula I oxalate; Formula I fumarate Form I; Formula I fumarate Form II; Formula I fumarate Form III; Formula I bis-malonate; and/or Formula I L-tartrate. Furthermore, the appearance of the phrase “a compound of Formula II” can include Formula II freebase Form I; Formula II freebase Form II; Formula II freebase Form III; Formula II freebase Form IV; Formula II freebase Form V; Formula II freebase Form VI; Formula II freebase Form VII; Formula II freebase Form VIII; Formula II freebase Form IX; and/or Formula II freebase Form X.

The disclosure disclosed herein is also meant to encompass all pharmaceutically acceptable compounds of Formula I being isotopically labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labeled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase, or dosage requirements may be reduced. Thus, heavier isotopes may be preferred in some circumstances.

Substitution with positron emitting isotopes, such as ¹¹C, ¹⁸F, ¹⁵ and ¹³N, can be useful in Positron Emission Topography (PET) studies for examining substrate receptor occupancy. Isotopically labeled compounds of Formula I can generally be prepared by conventional techniques known to those skilled in the art or by processes analogous to those described in the Examples as set out below using an appropriate isotopically labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, and/or emulsifier, or a combination of one or more of the above which has been approved by the United States Food and Drug Administration as being acceptable for use in humans or domestic animals.

A “pharmaceutical composition” refers to a formulation of a compound of the disclosure (e.g., a compound of Formula I) and a medium generally accepted in the art for the delivery of the biologically active compound to mammals, e.g., humans. Such a medium includes all pharmaceutically acceptable excipients thereof.

“Effective amount” or “therapeutically effective amount” refers to an amount of a compound according to the disclosure, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the disclosure which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the disclosure, and the age, body weight, general health, sex and diet of the patient. Such a therapeutically effective amount can be determined routinely by one of ordinary skill in the art having regard to their own knowledge, the state of the art, and this disclosure.

“Prevention” or “preventing” or “prophylaxis” means any treatment of a disease or condition that causes the clinical symptoms of the disease or condition not to develop. Compounds may, in some embodiments, be administered to a subject (including a human) who is at risk or has a family history of the disease or condition.

“Treating” and “treatment” of a disease include the following: preventing or reducing the risk of developing the disease, i.e., causing the clinical symptoms of the disease not to develop in a subject that may be exposed to or predisposed to the disease but does not yet experience or display symptoms of the disease, inhibiting the disease, i.e., arresting or reducing the development of the disease or its clinical symptoms, and relieving the disease, i.e., causing regression of the disease or its clinical symptoms.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats, and pigs), and/or laboratory animals (e.g., mice, rats, hamsters, guinea pigs, pigs, rabbits, dogs, and monkeys). In some embodiments, the subject (or the patient) is a human. “Human (or patient) in need thereof” refers to a human who may have or is suspected of having diseases or conditions that would benefit from certain treatment; for example, being treated with the compounds disclosed herein according to the present application.

Reference to “about” a value or parameter herein includes (and describes) embodiments that are directed to that value or parameter per se. For example, description referring to “about X” includes description of “X.” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Unit dosage forms” are physically discrete units suitable as unitary dosages for subjects (e.g., human subjects and other mammals), each unit containing a predetermined quantity of active material calculated to produce the desired therapeutic effect, in association with a suitable pharmaceutical excipient.

The term “substantially as shown in” when referring, for example, to an XRPD pattern, a DSC thermogram, a DVS isotherm, a TGA-MS thermogram, or a TGA thermogram includes a pattern, thermogram or spectrum that is not necessarily identical to those depicted herein, but that falls within the limits of experimental error or deviations when considered by one of ordinary skill in the art.

In some embodiments, the term “substantially pure” or “substantially free” with respect to a particular crystalline form of a compound means that the composition comprising the crystalline form contains less than 99%, less than 95%, less than 90%, less than 85%, less than 80%, less than 75%, less than 70%, less than 65%, less than 60%, less than 55%, less than 50%, less than 40%, less than 30%, less than 20%, less than 15%, less than 10%, less than 5%, or less than 1% by weight of other substances, including other crystalline forms and/or impurities. In certain embodiments, “substantially pure” or “substantially free of” refers to a substance free of other substances, including other crystalline forms and/or impurities. Impurities may, for example, include by-products or left-over reagents from chemical reactions, contaminants, degradation products, other crystalline forms, water, and solvents.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound. When the solvent is water, the “solvate” is a “hydrate.” A “solvate” may also be formed through interaction with ambient environment and starting material. Solvents are generally known to persons skilled in the art and can include, for example, methanol, ethanol, ethanol/water, acetone, tetrahydrofuran, dichloromethane, methyl t-butyl ether, 2-propanol, 1-propanol, cyclopentyl methyl ether, 2-methyltetrahydrofuran, methyl ethyl ketone, and monohydrate acetonitrile.

In certain embodiments, provided are optical isomers, racemates, or other mixtures thereof of the compounds described herein or a pharmaceutically acceptable salt or a mixture thereof. In some embodiments, isomers can be separated by methods well known in the art, e.g., liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposable mirror images of one another. “Diastereomers” are stereoisomers that have at least two asymmetric atoms, but which are not mirror-images of each other.

The compounds disclosed herein, and their pharmaceutically acceptable salts may include an asymmetric center and may thus give rise to enantiomers, diastereomers, and other stereoisomeric forms that may be defined, in terms of absolute stereochemistry, as (R) or (S) or, as (D) or (L) for amino acids. The present invention is meant to include all such possible isomers, as well as their racemic and optically pure forms. Optically active (+) and ( ), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Disclosed herein are solid forms of an α4β7 integrin inhibitor. Also disclosed herein are compounds useful for the synthesis of an α4β7 integrin inhibitor.

### Solid Forms of Formula I

Solid forms of Formula I, including crystalline forms and substantially pure forms, may provide the advantage of bioavailability and stability, suitable for use and an active ingredient in a pharmaceutical composition. Development of a suitable solid form for use in pharmaceutical compositions requires considerations of stability and bioavailability in varying environments. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, solid forms of the compound of Formula I may provide advantages such as improving: the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different solid forms of the compound of Formula I described herein which may exhibit one or more favorable characteristics described above. The process for the preparation of the solid forms described herein and characterization of these solid forms are described in detail below.

In particular embodiments, novel solid forms, such as crystalline forms of Formula I are disclosed. In some embodiments, a solid form of Formula I freebase Form I is disclosed. In some embodiments, a solid form of Formula I freebase acetophenone solvate is disclosed. In some embodiments, a solid form of Formula I freebase anisole solvate is disclosed. In some embodiments, a solid form of Formula I gentisate Form I is disclosed. In some embodiments, a solid form of Formula I mono-malonate monohydrate acetonitrile solvate is disclosed. In some embodiments, a solid form of Formula I mono-malonate Form I is disclosed. In some embodiments a solid form of Formula I mono-malonate Form II is disclosed. In some embodiments, a solid form of Formula I mono-malonate acetone solvate is disclosed. In some embodiments, a solid form of Formula I mono-malonate 2-methyltetrahydrofuran solvate is disclosed. In some embodiments, a solid form of Formula I mono-malonate tetrahydrofuran solvate is disclosed. In some embodiments, a solid form of Formula I xinafoate Form I is disclosed. In some embodiments, a solid form of Formula I xinafoate Form II is disclosed. In some embodiments, a solid form of Formula I oxalate is disclosed. In some embodiments, a solid form Formula I fumarate Form I is disclosed. In some embodiments, a solid form of Formula I fumarate Form II is disclosed. In some embodiments, a solid form of Formula I fumarate Form III is disclosed. In some embodiments, a solid form of Formula I bis-malonate is disclosed. In some embodiments, a solid form of Formula I L-tartrate is disclosed.

### Formula I Freebase Form I

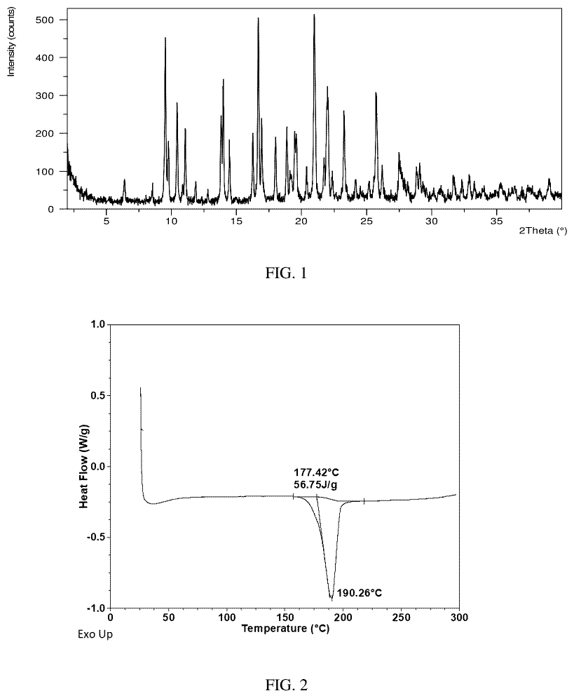

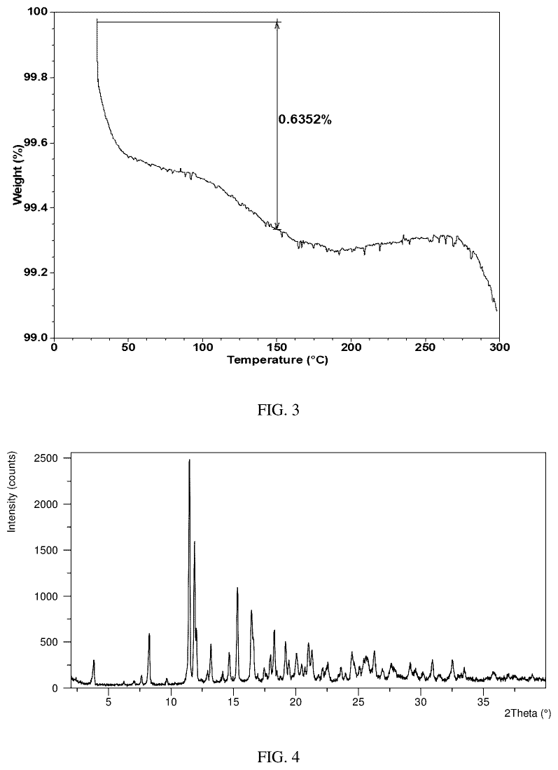

In some embodiments, provided herein is a Formula I freebase Form I, wherein the solid form exhibits an X-ray powder diffraction (XRPD) pattern substantially as shown in FIG. 1. Formula I freebase Form I may exhibit a differential scanning calorimetry (DSC) thermogram substantially as shown in FIG. 2. Formula I freebase Form I may exhibit a thermogravimetric analysis (TGA) thermogram substantially as shown in FIG. 3.

In some embodiments of crystalline Formula I freebase Form I, at least one, at least two, or all of the following (a)-(c) apply: (a) a crystalline Formula I freebase Form I has an XRPD pattern substantially as shown in FIG. 1; (b) a crystalline Formula I freebase Form I has a DSC thermogram substantially as shown in FIG. 2; (c) crystalline Formula I freebase Form I has a TGA thermogram substantially as shown in FIG. 3.

In some embodiments, crystalline Formula I freebase Form I has an XRPD pattern displaying at least two, at least three, at least four, at least five, at least six, at least seven, at least eight, or at least nine of the degree 20-reflections with the greatest intensity as the XRPD pattern substantially as shown in FIG. 1.

In some embodiments, Formula I freebase Form I has an XRPD pattern comprising degree 2θ-reflections (±0.2 degrees 2θ) at 9.5, 21.0, and 25.7 degrees.

In some embodiments, Formula I freebase Form I has an XRPD pattern comprising degree 2θ-reflections (±0.2 degrees 2θ) at 9.5, 21.0, and 25.7 degrees and one, two, or three of the degree 2θ-reflections (±0.2 degrees 2θ) at 10.5, 16.7, and 22.0 degrees.

In some embodiments, Formula I freebase Form I has an XRPD pattern comprising degree 2θ-reflections (±0.2 degrees 2θ) at 9.5, 21.0, and 25.7 degrees and one, two or three of the degree 2θ-reflections (±0.2 degrees 2θ) at 11.1, 13.8, and 23.3 degrees.

In some embodiments, Formula I freebase Form I has an XRPD pattern comprising degree 2θ-reflections (±0.2 degrees 2θ) at 9.5, 21.0, 25.7, 10.5, 16.7, 22.0, 11.1, 13.8, and 23.3 degrees.

In some embodiments, Formula I freebase Form I has a differential scanning calorimetry thermogram having an endothermic transition with an onset at about 177 °C.

### Formula I Freebase Acetophenone Solvate

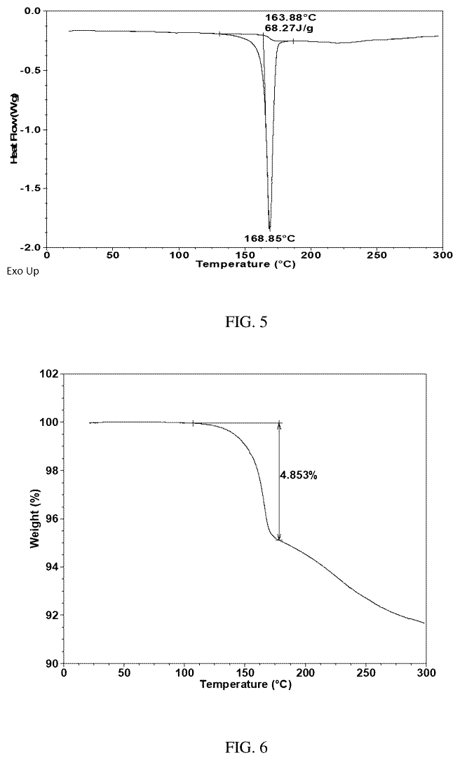

In some embodiments, provided herein is a crystalline Formula I freebase acetophenone solvate (Formula I freebase acetophenone solvate), wherein the crystalline structure exhibits an X-Ray powder diffraction (XRPD) pattern substantially as shown in FIG. 4. Formula I freebase acetophenone solvate may exhibit a differential scanning calorimetry (DSC) thermogram substantially as shown in FIG. 5. Crystalline Formula I freebase acetophenone solvate has a thermogravimetric analysis (TGA) thermogram substantially as shown in FIG. 6.

In some embodiments of crystalline Formula I freebase acetophenone solvate, at least one, at least two, or all of the following (a)-(c) apply: (a) a crystalline Formula I freebase acetophenone solvate has an XRPD pattern substantially as shown in FIG. 4; (b) a crystalline Formula I freebase acetophenone solvate has a DSC thermogram substantially as shown in FIG. 5; (c) crystalline Formula I freebase acetophenone solvate has a TGA thermogram substantially as shown in FIG. 6.

In some embodiments, crystalline Formula I freebase acetophenone solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 4.

In some embodiments, Formula I freebase acetophenone solvate has an XRPD pattern comprising degree 2θ-reflections (±0.2 degrees 2θ) at 8.3, 11.5, and 15.3 degrees.

In some embodiments, Formula I freebase acetophenone solvate has an XRPD pattern comprising degree 2θ-reflections (±0.2 degrees 2θ) at 11.9, 16.4, and 18.3 degrees and one, two, or three of the degree 2θ-reflections (±0.2 degrees 2θ) at 8.3, 11.5, and 15.3 degrees.

In some embodiments, Formula I freebase acetophenone solvate has an XRPD pattern comprising degree 2θ-reflections (±0.2 degrees 2θ) at 12.0, 16.6, and 21.0 degrees and one, two or three of the degree 2θ-reflections (±0.2 degrees 2θ) at 8.3, 11.5, and 15.3 degrees.

In some embodiments, Formula I freebase acetophenone solvate has an XRPD pattern comprising degree 2θ-reflections (±0.2 degrees 2θ) at 8.3, 11.5, 15.3, 11.9, 16.4, 18.3, 12.0, 16.6, and 21.0 degrees.

In some embodiments, Formula I freebase acetophenone solvate has a differential scanning calorimetry thermogram having an endothermic transition with an onset at about 164 °C.

### Formula I Freebase Anisole Solvate

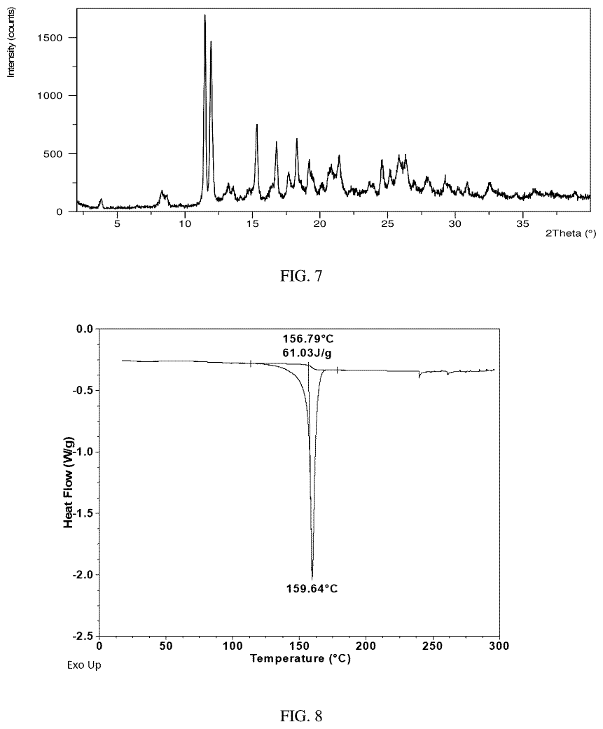

In some embodiments, provided herein is a crystalline Formula I freebase anisole solvate (Formula I freebase anisole solvate), wherein the crystalline structure exhibits an X-Ray powder diffraction (XRPD) pattern substantially as shown in FIG. 7. Formula I freebase anisole solvate may exhibit a differential scanning calorimetry (DSC) thermogram substantially as shown in FIG. 8. Crystalline Formula I freebase anisole solvate has a thermogravimetric analysis (TGA) thermogram substantially as shown in FIG. 9.

In some embodiments of crystalline Formula I freebase anisole solvate, at least one, at least two, or all of the following (a)-(c) apply: (a) a crystalline Formula I freebase anisole solvate has an XRPD pattern substantially as shown in FIG. 7; (b) a crystalline Formula I freebase anisole solvate has a DSC thermogram substantially as shown in FIG. 8; (c) crystalline Formula I freebase anisole solvate has a TGA thermogram substantially as shown in FIG. 9.

In some embodiments, crystalline Formula I freebase anisole solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 7.

In some embodiments, Formula I freebase anisole solvate has an XRPD pattern comprising degree 2θ-reflections (±0.2 degrees 2θ) at 11.5, 15.4, and 18.3 degrees.

In some embodiments, Formula I freebase anisole solvate has an XRPD pattern comprising degree 2θ-reflections (±0.2 degrees 2θ) at 11.9, 16.8, and 21.4 degrees and one, two, or three of the degree 2θ-reflections (±0.2 degrees 2θ) at 11.5, 15.4, and 18.3 degrees.

In some embodiments, Formula I freebase anisole solvate has an XRPD pattern comprising degree 2θ-reflections (±0.2 degrees 2θ) at 17.6, 19.2, and 24.6 degrees and one, two or three of the degree 2θ-reflections (±0.2 degrees 2θ) at 11.5, 15.4, and 18.3 degrees.

In some embodiments, Formula I freebase anisole solvate has an XRPD pattern comprising degree 2θ-reflections (±0.2 degrees 2θ) at 11.5, 15.4, 18.3, 11.9, 16.8, 21.4, 17.6, 19.2, and 24.6 degrees.

In some embodiments, Formula I freebase anisole solvate has a differential scanning calorimetry thermogram having an endothermic transition with an onset at about 157 °C.

### Formula I Gentisate Form I

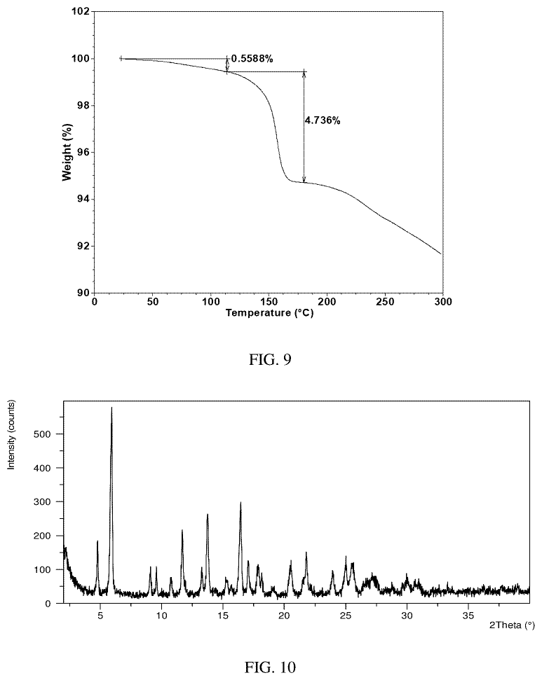

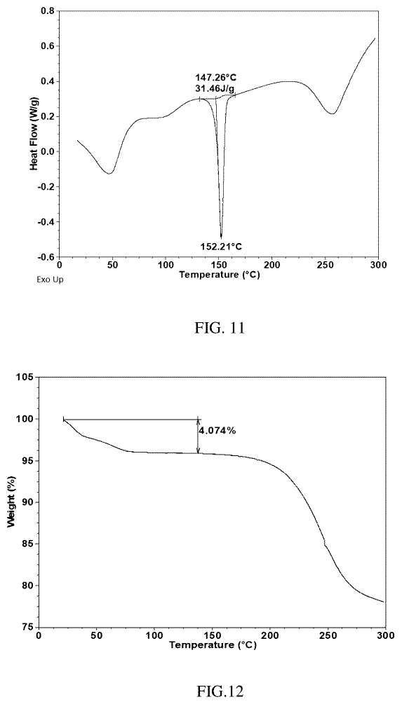

In some embodiments, provided herein is a crystalline Formula I gentisate Form I (Formula I gentisate Form I), wherein the crystalline structure exhibits an X-Ray powder diffraction (XRPD) pattern substantially as shown in FIG. 10. Formula I gentisate Form I may exhibit a differential scanning calorimetry (DSC) thermogram substantially as shown in FIG. 11. Crystalline Formula I gentisate Form I has a thermogravimetric analysis (TGA) thermogram substantially as shown in FIG. 12.

In some embodiments of crystalline Formula I gentisate Form I, at least one, at least two, or all of the following (a)-(c) apply: (a) a crystalline Formula I gentisate Form I has an XRPD pattern substantially as shown in FIG. 10; (b) a crystalline Formula I gentisate Form I has a DSC thermogram substantially as shown in FIG. 11; (c) crystalline Formula I gentisate Form I has a TGA thermogram substantially as shown in FIG. 12.

In some embodiments, crystalline Formula I gentisate Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 10.

In some embodiments, Formula I gentisate Form I has an XRPD pattern comprising degree 2θ-reflections (±0.2 degrees 2θ) at 5.9, 16.4, and 21.8 degrees.

In some embodiments, Formula I gentisate Form I has an XRPD pattern comprising degree 2θ-reflections (±0.2 degrees 2θ) at 4.8, 13.8, and 17.1 degrees and one, two, or three of the degree 2θ-reflections (±0.2 degrees 2θ) at 5.9, 16.4, and 21.8 degrees.

In some embodiments, Formula I gentisate Form I has an XRPD pattern comprising degree 2θ-reflections (±0.2 degrees 2θ) at 9.6, 11.7, and 25.0 degrees and one, two or three of the degree 2θ-reflections (±0.2 degrees 2θ) at 5.9, 16.4, and 21.8 degrees.

In some embodiments, Formula I gentisate Form I has an XRPD pattern comprising degree 2θ-reflections (±0.2 degrees 2θ) at 5.9, 16.4, 21.8, 4.8, 13.8, 17.1, 9.6, 11.7, and 25.0 degrees.

In some embodiments, Formula I gentisate Form I has a differential scanning calorimetry thermogram having an endothermic transition with an onset at about 147 °C.

### Formula I Mono-Malonate Monohydrate Acetonitrile Solvate

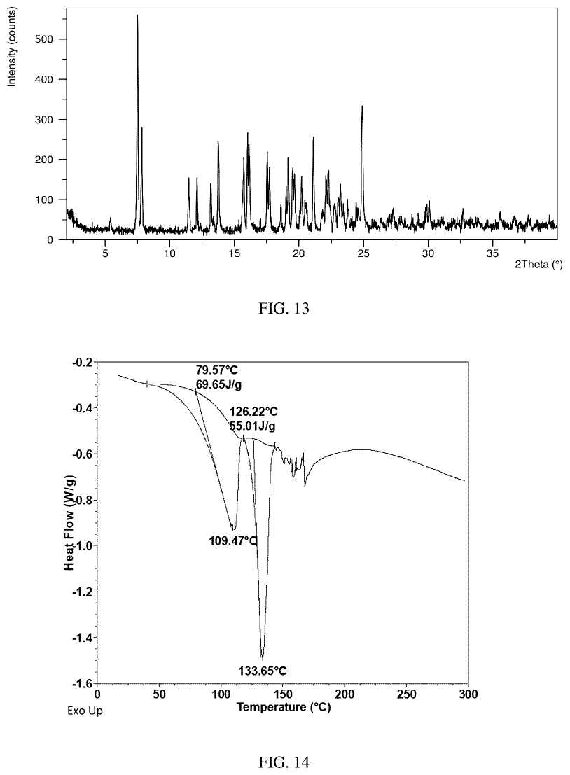

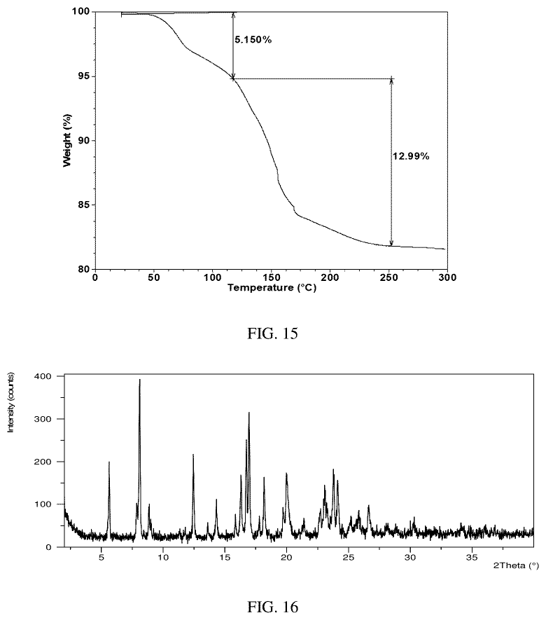

In some embodiments, provided herein is a crystalline Formula I mono-malonate acetonitrile solvate (Formula I mono-malonate acetonitrile solvate), wherein the crystalline structure exhibits an X-Ray powder diffraction (XRPD) pattern substantially as shown in FIG. 13. Formula I mono-malonate acetonitrile solvate may exhibit a differential scanning calorimetry (DSC) thermogram substantially as shown in FIG. 14. Crystalline Formula I mono-malonate acetonitrile solvate has a thermogravimetric analysis (TGA) thermogram substantially as shown in FIG. 15.

In some embodiments of crystalline Formula I mono-malonate acetonitrile solvate, at least one, at least two, or all of the following (a)-(c) apply: (a) a crystalline Formula I mono-malonate acetonitrile solvate has an XRPD pattern substantially as shown in FIG. 13; (b) a crystalline Formula I mono-malonate acetonitrile solvate has a DSC thermogram substantially as shown in FIG. 14; (c) crystalline Formula I mono-malonate acetonitrile solvate has a TGA thermogram substantially as shown in FIG. 15.

In some embodiments, crystalline Formula I mono-malonate acetonitrile solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 13.

In some embodiments, Formula I mono-malonate acetonitrile solvate has an XRPD pattern comprising degree 2θ-reflections (±0.2 degrees 2θ) at 7.5, 13.8, and 16.0 degrees.

In some embodiments, Formula I mono-malonate acetonitrile solvate has an XRPD pattern comprising degree 2θ-reflections (±0.2 degrees 2θ) at 7.8, 12.1, and 19.2 degrees and one, two, or three of the degree 2θ-reflections (±0.2 degrees 2θ) at 7.5, 13.8, and 16.0 degrees.

In some embodiments, Formula I mono-malonate acetonitrile solvate has an XRPD pattern comprising degree 2θ-reflections (±0.2 degrees 2θ) at 11.5, 17.5, and 21.1 degrees and one, two or three of the degree 2θ-reflections (±0.2 degrees 2θ) at 7.5, 13.8, and 16.0 degrees.

In some embodiments, Formula I mono-malonate acetonitrile solvate has an XRPD pattern comprising degree 2θ-reflections (±0.2 degrees 2θ) at 5.9, 16.4, 21.8, 4.8, 13.8, 17.1, 9.6, 11.7, and 21.1 degrees.

In some embodiments, Formula I mono-malonate acetonitrile solvate has a differential scanning calorimetry thermogram having a first endothermic transition with an onset at about 80 °C, and a second endothermic transition with an onset at about 126 °C.

### Formula I Mono-Malonate Form I

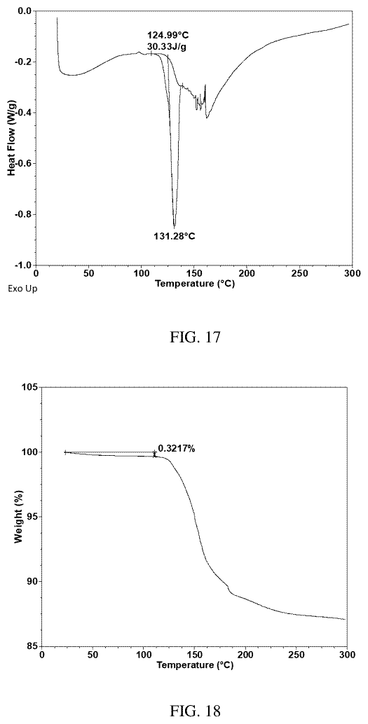

In some embodiments, provided herein is a crystalline Formula I mono-malonate Form I (Formula I mono-malonate Form I), wherein the crystalline structure exhibits an X-Ray powder diffraction (XRPD) pattern substantially as shown in FIG. 16. Formula I mono-malonate Form I may exhibit a differential scanning calorimetry (DSC) thermogram substantially as shown in FIG. 17. Crystalline Formula I mono-malonate Form I has a thermogravimetric analysis (TGA) thermogram substantially as shown in FIG. 18.

In some embodiments of crystalline Formula I mono-malonate Form I, at least one, at least two, or all of the following (a)-(c) apply: (a) a crystalline Formula I mono-malonate Form I has an XRPD pattern substantially as shown in FIG. 16; (b) a crystalline Formula I mono-malonate Form I has a DSC thermogram substantially as shown in FIG. 17; (c) crystalline Formula I mono-malonate Form I has a TGA thermogram substantially as shown in FIG. 18.

In some embodiments, crystalline Formula I mono-malonate Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 16.

In some embodiments, Formula I mono-malonate Form I has an XRPD pattern comprising degree 2θ-reflections (±0.2 degrees 2θ) at 8.1, 12.4, and 17.0 degrees.

In some embodiments, Formula I mono-malonate Form I has an XRPD pattern comprising degree 2θ-reflections (±0.2 degrees 2θ) at 5.6, 16.7, and 20.0 degrees and one, two, or three of the degree 2θ-reflections (±0.2 degrees 2θ) at 8.1, 12.4, and 17.0 degrees.

In some embodiments, Formula I mono-malonate Form I has an XRPD pattern comprising degree 2θ-reflections (±0.2 degrees 2θ) at 8.9, 16.3, and 23.8 degrees and one, two or three of the degree 2θ-reflections (±0.2 degrees 2θ) at 8.1, 12.4, and 17.0 degrees.

In some embodiments, Formula I mono-malonate Form I has an XRPD pattern comprising degree 2θ-reflections (±0.2 degrees 2θ) at 8.1, 12.4, 17.0, 5.6, 16.7 20.0, 8.9, 16.3, and 23.8 degrees.

In some embodiments, Formula I mono-malonate Form I has a differential scanning calorimetry thermogram having an endothermic transition with an onset at about 125 °C.

### Formula I Mono-Malonate Form II

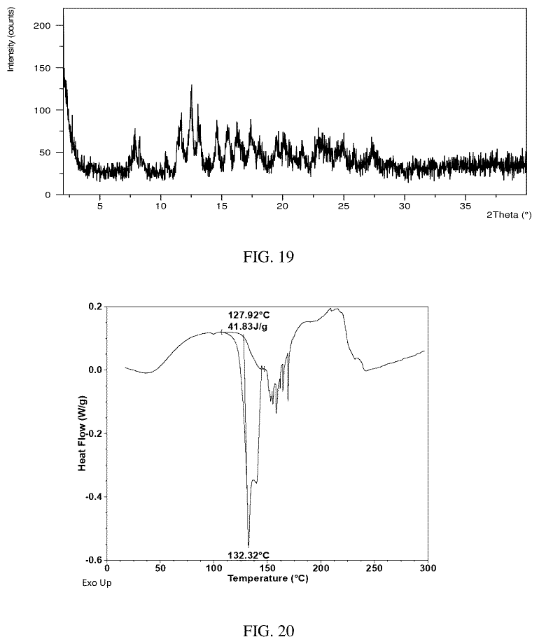

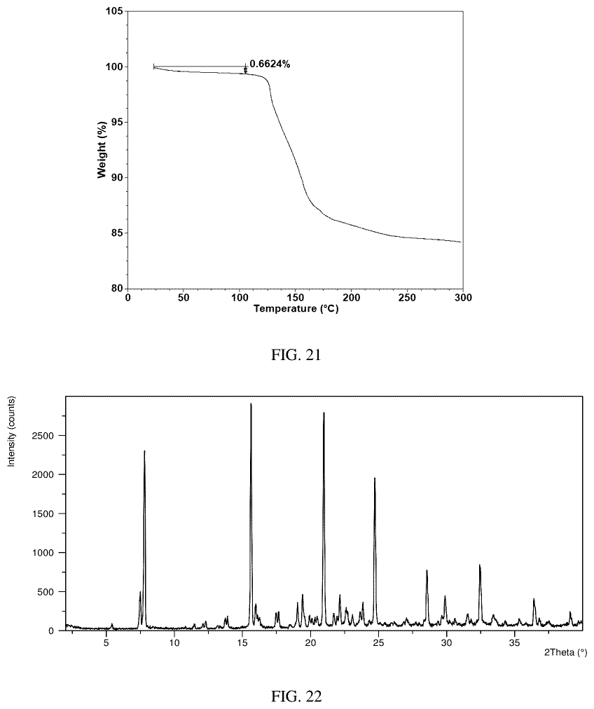

In some embodiments, provided herein is a crystalline Formula I mono-malonate Form II (Formula I mono-malonate Form II), wherein the crystalline structure exhibits an X-Ray powder diffraction (XRPD) pattern substantially as shown in FIG. 19. Formula I mono-malonate Form II may exhibit a differential scanning calorimetry (DSC) thermogram substantially as shown in FIG. 20. Crystalline Formula I mono-malonate Form II has a thermogravimetric analysis (TGA) thermogram substantially as shown in FIG. 21.

In some embodiments of crystalline Formula I mono-malonate Form II, at least one, at least two, or all of the following (a)-(c) apply: (a) a crystalline Formula I mono-malonate Form II has an XRPD pattern substantially as shown in FIG. 19; (b) a crystalline Formula I mono-malonate Form II has a DSC thermogram substantially as shown in FIG. 20; (c) crystalline Formula I mono-malonate Form II has a TGA thermogram substantially as shown in FIG. 21.

In some embodiments, crystalline Formula I mono-malonate Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 19.

In some embodiments, Formula I mono-malonate Form II has an XRPD pattern comprising degree 2θ-reflections (±0.2 degrees 2θ) at 12.5, 13.1, and 15.5 degrees.

In some embodiments, Formula I mono-malonate Form II has an XRPD pattern comprising degree 2θ-reflections (±0.2 degrees 2θ) at 11.6, 14.6, and 16.2 degrees and one, two, or three of the degree 2θ-reflections (±0.2 degrees 2θ) at 12.5, 13.1, and 15.5 degrees.

In some embodiments, Formula I mono-malonate Form II has an XRPD pattern comprising degree 2θ-reflections (±0.2 degrees 2θ) at 7.9, 17.4, and 23.0 degrees and one, two or three of the degree 2θ-reflections (±0.2 degrees 2θ) at 12.5, 13.1, and 15.5 degrees.

In some embodiments, Formula I mono-malonate Form II has an XRPD pattern comprising degree 2θ-reflections (±0.2 degrees 2θ) at 12.5, 13.1, 15.5, 11.6, 14.6, 16.2, 7.9, 17.4, and 23.0 degrees.

In some embodiments, Formula I mono-malonate Form II has a differential scanning calorimetry thermogram having an endothermic transition with an onset at about 128 °C.

### Formula I Mono-Malonate Acetone Solvate

In some embodiments, provided herein is a crystalline Formula I mono-malonate acetone solvate (Formula I mono-malonate acetone solvate), wherein the crystalline structure exhibits an X-Ray powder diffraction (XRPD) pattern substantially as shown in FIG. 22.

In some embodiments, crystalline Formula I mono-malonate acetone solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 22.

In some embodiments, Formula I mono-malonate acetone solvate has an XRPD pattern comprising degree 2θ-reflections (±0.2 degrees 2θ) at 7.5, 7.8, and 15.6 degrees.

In some embodiments, Formula I mono-malonate acetone solvate has an XRPD pattern comprising degree 2θ-reflections (±0.2 degrees 2θ) at 12.3, 13.9, and 16.0 degrees and one, two, or three of the degree 2θ-reflections (±0.2 degrees 2θ) at 7.5, 7.8, and 15.6 degrees.

In some embodiments, Formula I mono-malonate acetone solvate has an XRPD pattern comprising degree 2θ-reflections (±0.2 degrees 2θ) at 17.7, 19.4, and 21.0 degrees and one, two or three of the degree 2θ-reflections (±0.2 degrees 2θ) at 7.5, 7.8, and 15.6 degrees.

In some embodiments, Formula I mono-malonate acetone solvate has an XRPD pattern comprising degree 2θ-reflections (±0.2 degrees 2θ) at 7.5, 7.8, 15.6, 12.3, 13.9, 16.0, 17.7, 19.4, and 21.0 degrees.

### Formula I Mono-Malonate 2-Methyltetrahydrofuran Solvate

In some embodiments, provided herein is a crystalline Formula I mono-malonate 2-methyltetrahydrofuran solvate (Formula I mono-malonate 2-methyltetrahydrofuran solvate), wherein the crystalline structure exhibits an X-Ray powder diffraction (XRPD) pattern substantially as shown in FIG. 23.

In some embodiments, crystalline Formula I mono-malonate 2-methyltetrahydrofuran solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 23.

In some embodiments, Formula I mono-malonate 2-methyltetrahydrofuran solvate has an XRPD pattern comprising degree 2θ-reflections (±0.2 degrees 2θ) at 7.4, 12.0, and 13.6 degrees.

In some embodiments, Formula I mono-malonate 2-methyltetrahydrofuran solvate has an XRPD pattern comprising degree 2θ-reflections (±0.2 degrees 2θ) at 7.7, 11.3, and 15.5 degrees and one, two, or three of the degree 2θ-reflections (±0.2 degrees 2θ) at 7.4, 12.0, and 13.6 degrees.

In some embodiments, Formula I mono-malonate 2-methyltetrahydrofuran solvate has an XRPD pattern comprising degree 2θ-reflections (±0.2 degrees 2θ) at 15.9, 18.3, and 20.8 degrees and one, two or three of the degree 2θ-reflections (±0.2 degrees 2θ) at 7.4, 12.0, and 13.6 degrees.

In some embodiments, Formula I mono-malonate 2-methyltetrahydrofuran solvate has an XRPD pattern comprising degree 2θ-reflections (±0.2 degrees 2θ) at 7.4, 12.0, 13.6, 7.7, 11.3, 15.5, 15.9, 18.3, and 20.8 degrees.

### Formula I Mono-Malonate Tetrahydrofuran Solvate

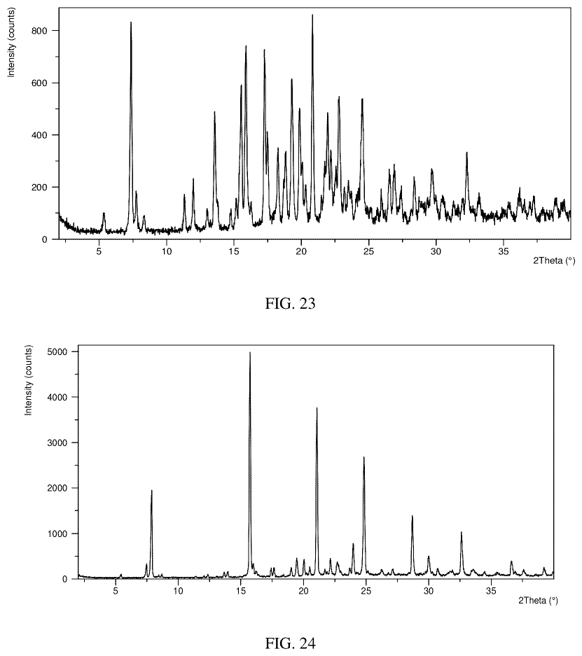

In some embodiments, provided herein is a crystalline Formula I mono-malonate tetrahydrofuran solvate (Formula I mono-malonate tetrahydrofuran solvate), wherein the crystalline structure exhibits an X-Ray powder diffraction (XRPD) pattern substantially as shown in FIG. 24.

In some embodiments, crystalline Formula I mono-malonate tetrahydrofuran solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 24.

In some embodiments, Formula I mono-malonate tetrahydrofuran solvate has an XRPD pattern comprising degree 2θ-reflections (±0.2 degrees 2θ) at 7.9, 15.7, and 21.1 degrees.

In some embodiments, Formula I mono-malonate tetrahydrofuran solvate has an XRPD pattern comprising degree 2θ-reflections (±0.2 degrees 2θ) at 7.5, 13.9, and 19.4, degrees and one, two, or three of the degree 2θ-reflections (±0.2 degrees 2θ) at 7.9, 15.7, and 21.1 degrees.

In some embodiments, Formula I mono-malonate tetrahydrofuran solvate has an XRPD pattern comprising degree 2θ-reflections (±0.2 degrees 2θ) at 13.7, 17.4, and 20.0 degrees and one, two or three of the degree 2θ-reflections (±0.2 degrees 2θ) at 7.9, 15.7, and 21.1 degrees.

In some embodiments, Formula I mono-malonate tetrahydrofuran solvate has an XRPD pattern comprising degree 2θ-reflections (±0.2 degrees 2θ) at 7.9, 15.7, 21.1, 7.5, 13.9, 19.4, 13.7, 17.4, and 20.0 degrees.

### Formula I Xinafoate Form I

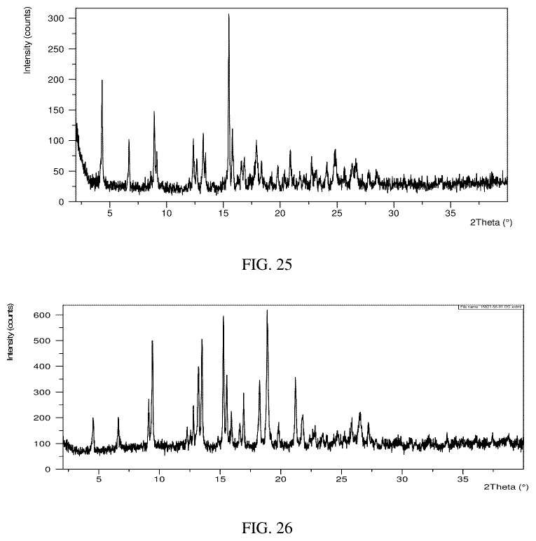

In some embodiments, provided herein is a crystalline Formula I xinafoate Form I (Formula I xinafoate Form I), wherein the crystalline structure exhibits an X-Ray powder diffraction (XRPD) pattern substantially as shown in FIG. 25.

In some embodiments, crystalline Formula I xinafoate Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 25.

In some embodiments, Formula I xinafoate Form I has an XRPD pattern comprising degree 2θ-reflections (±0.2 degrees 2θ) at 4.3, 6.7, and 15.5 degrees.

In some embodiments, Formula I xinafoate Form I has an XRPD pattern comprising degree 2θ-reflections (±0.2 degrees 2θ) at 8.9, 13.2, and 15.8 degrees and one, two, or three of the degree 2θ-reflections (±0.2 degrees 2θ) at 4.3, 6.7, and 15.5 degrees.

In some embodiments, Formula I xinafoate Form I has an XRPD pattern comprising degree 2θ-reflections (±0.2 degrees 2θ) at 17.9, 20.9, and 24.8 degrees and one, two or three of the degree 2θ-reflections (±0.2 degrees 2θ) at 4.3, 6.7, and 15.5 degrees.

In some embodiments, Formula I xinafoate Form I has an XRPD pattern comprising degree 2θ-reflections (±0.2 degrees 2θ) at 4.3, 6.7, 15.5, 8.9, 13.2, 15.8, 17.9, 20.9, and 24.8 degrees.

### Formula I Xinafoate Form II

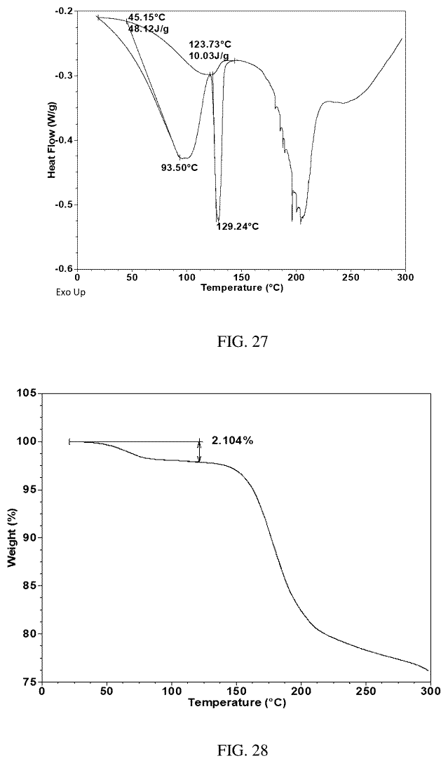

In some embodiments, provided herein is a crystalline Formula I xinafoate Form II (Formula I xinafoate Form II), wherein the crystalline structure exhibits an X-Ray powder diffraction (XRPD) pattern substantially as shown in FIG. 26. Formula I xinafoate Form II may exhibit a differential scanning calorimetry (DSC) thermogram substantially as shown in FIG. 27. Crystalline Formula I xinafoate Form II has a thermogravimetric analysis (TGA) thermogram substantially as shown in FIG. 28.

In some embodiments of crystalline Formula I xinafoate Form II, at least one, at least two, or all of the following (a)-(c) apply: (a) a crystalline Formula I xinafoate Form II has an XRPD pattern substantially as shown in FIG. 26; (b) a crystalline Formula I xinafoate Form II has a DSC thermogram substantially as shown in FIG. 27; (c) crystalline Formula I xinafoate Form II has a TGA thermogram substantially as shown in FIG. 28.

In some embodiments, crystalline Formula I xinafoate Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 26.

In some embodiments, Formula I xinafoate Form II has an XRPD pattern comprising degree 2θ-reflections (±0.2 degrees 2θ) at 9.4, 15.3, and 18.9 degrees.

In some embodiments, Formula I xinafoate Form II has an XRPD pattern comprising degree 2θ-reflections (±0.2 degrees 2θ) at 4.6, 13.5, and 16.9 degrees and one, two, or three of the degree 2θ-reflections (±0.2 degrees 2θ) at 9.4, 15.3, and 18.9 degrees.

In some embodiments, Formula I xinafoate Form II has an XRPD pattern comprising degree 2θ-reflections (±0.2 degrees 2θ) at 6.6, 13.2, and 21.2 degrees and one, two or three of the degree 2θ-reflections (±0.2 degrees 2θ) at 9.4, 15.3, and 18.9 degrees.

In some embodiments, Formula I xinafoate Form II has an XRPD pattern comprising degree 2θ-reflections (±0.2 degrees 2θ) at 9.4, 15.3, 18.9, 4.6, 13.5, 16.9, 6.6, 13.2, and 21.2 degrees.

In some embodiments, Formula I xinafoate Form II has a differential scanning calorimetry thermogram having an endothermic transition with an onset at about 45 °C. In some embodiments, Formula I xinafoate Form II has a differential scanning calorimetry thermogram having an endothermic transition with an onset at about 124 °C. In some embodiments, Formula I xinafoate Form II has a differential scanning calorimetry thermogram having an endothermic transition with an onset at about 45 °C, and a second endothermic transition with an onset at about 124 °C.

### Formula I Oxalate

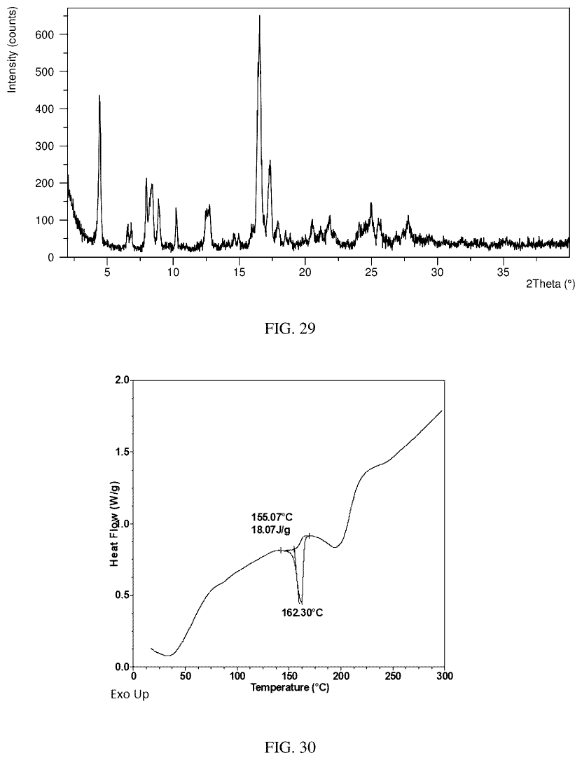

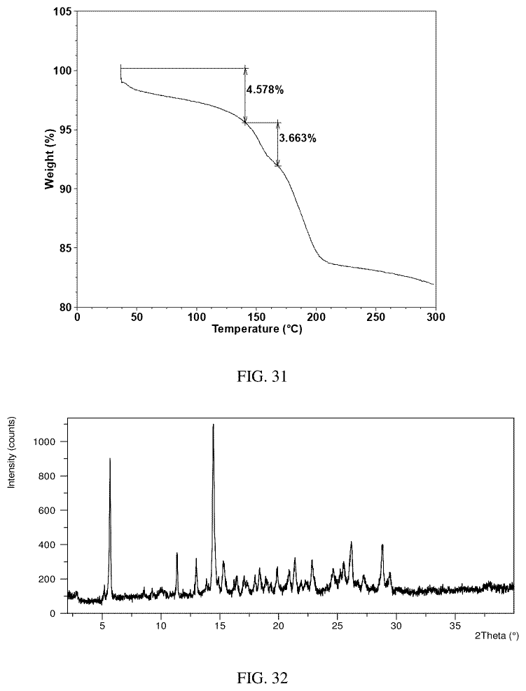

In some embodiments, provided herein is a crystalline Formula I oxalate (Formula I oxalate), wherein the crystalline structure exhibits an X-Ray powder diffraction (XRPD) pattern substantially as shown in FIG. 29. Formula I oxalate may exhibit a differential scanning calorimetry (DSC) thermogram substantially as shown in FIG. 30. Crystalline Formula I oxalate has a thermogravimetric analysis (TGA) thermogram substantially as shown in FIG. 31.

In some embodiments of crystalline Formula I oxalate, at least one, at least two, or all of the following (a)-(c) apply: (a) a crystalline Formula I oxalate Form I has an XRPD pattern substantially as shown in FIG. 29; (b) a crystalline Formula I oxalate has a DSC thermogram substantially as shown in FIG. 30; (c) crystalline Formula I oxalate has a TGA thermogram substantially as shown in FIG. 31.

In some embodiments, crystalline Formula I oxalate has an XRPD pattern displaying at least two, at least three, at least four, at least five, at least six, at least seven, at least eight, or at least nine of the degree 2θ-reflections with the greatest intensity as the XRPD pattern substantially as shown in FIG. 29.

In some embodiments, Formula I oxalate has an XRPD pattern comprising degree 2θ-reflections (±0.2 degrees 2θ) at 4.4, 8.0, and 16.5 degrees.

In some embodiments, Formula I oxalate has an XRPD pattern comprising degree 2θ-reflections (±0.2 degrees 2θ) at 6.8, 8.4, and 10.2 degrees and one, two, or three of the degree 2θ-reflections (±0.2 degrees 2θ) at 4.4, 8.0, and 16.5 degrees.

In some embodiments, Formula I oxalate has an XRPD pattern comprising degree 2θ-reflections (±0.2 degrees 2θ) at 6.5, 8.9, and 12.8 degrees and one, two or three of the degree 2θ-reflections (±0.2 degrees 2θ) at 4.4, 8.0, and 16.5 degrees.

In some embodiments, Formula I oxalate has an XRPD pattern comprising degree 2θ-reflections (±0.2 degrees 2θ) at 4.4, 8.0, 16.5, 6.8, 8.4, 10.2, 6.5, 8.9, and 12.8 degrees.

In some embodiments, Formula I oxalate has a differential scanning calorimetry thermogram having an endothermic transition with an onset at about 155 °C.

### Formula I Fumarate Form I

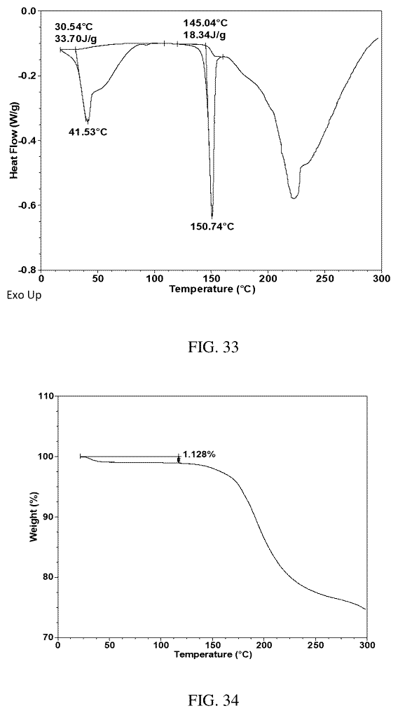

In some embodiments, provided herein is a crystalline Formula I fumarate Form I (Formula I fumarate Form I), wherein the crystalline structure exhibits an X-Ray powder diffraction (XRPD) pattern substantially as shown in FIG. 32. Formula I fumarate Form I may exhibit a differential scanning calorimetry (DSC) thermogram substantially as shown in FIG. 33. Crystalline Formula I fumarate Form I has a thermogravimetric analysis (TGA) thermogram substantially as shown in FIG. 34.

In some embodiments of crystalline Formula I fumarate Form I, at least one, at least two, or all of the following (a)-(c) apply: (a) a crystalline Formula I fumarate Form I has an XRPD pattern substantially as shown in FIG. 32; (b) a crystalline Formula I fumarate Form I has a DSC thermogram substantially as shown in FIG. 33; (c) crystalline Formula I fumarate Form I has a TGA thermogram substantially as shown in FIG. 34.

In some embodiments, crystalline Formula I fumarate Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 32.

In some embodiments, Formula I fumarate Form I has an XRPD pattern comprising degree 2θ-reflections (±0.2 degrees 2θ) at 5.7, 11.4, and 14.4 degrees.

In some embodiments, Formula I fumarate Form I has an XRPD pattern comprising degree 2θ-reflections (±0.2 degrees 2θ) at 13.0, 21.4, and 26.2 degrees and one, two, or three of the degree 2θ-reflections (±0.2 degrees 2θ) at 5.7, 11.4, and 14.4 degrees.

In some embodiments, Formula I fumarate Form I has an XRPD pattern comprising degree 2θ-reflections (±0.2 degrees 2θ) at 15.3, 18.4, and 28.8 degrees and one, two or three of the degree 2θ-reflections (±0.2 degrees 2θ) at 5.7, 11.4, and 14.4 degrees.

In some embodiments, Formula I fumarate Form I has an XRPD pattern comprising degree 2θ-reflections (±0.2 degrees 2θ) at 5.7, 11.4, 14.4, 13.0, 21.4, 26.2, 15.3, 18.4, and 28.8 degrees.

In some embodiments, Formula I fumarate Form I has a differential scanning calorimetry thermogram having an endothermic transition with an onset at about 31 °C. In some embodiments, Formula I fumarate Form I has a differential scanning calorimetry thermogram having an endothermic transition with an onset at about 145 °C. In some embodiments, Formula I fumarate Form I has a differential scanning calorimetry thermogram having an endothermic transition with an onset at about 31 °C, and a second endothermic transition with an onset at about 145 °C.

### Formula I Fumarate Form II

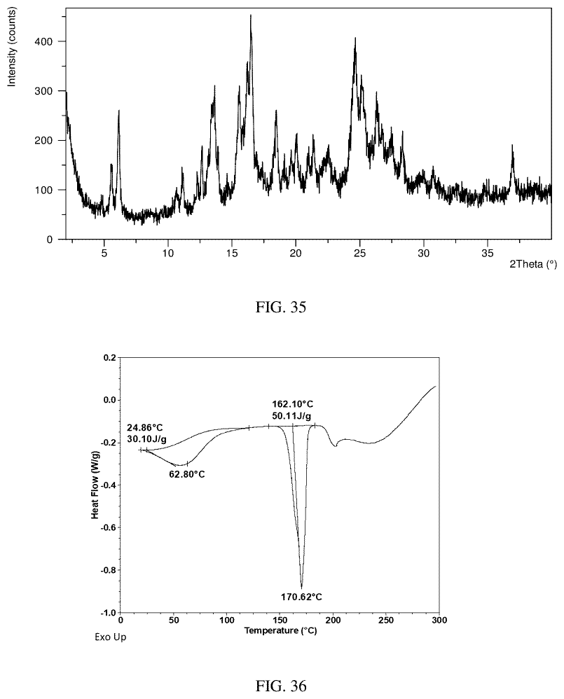

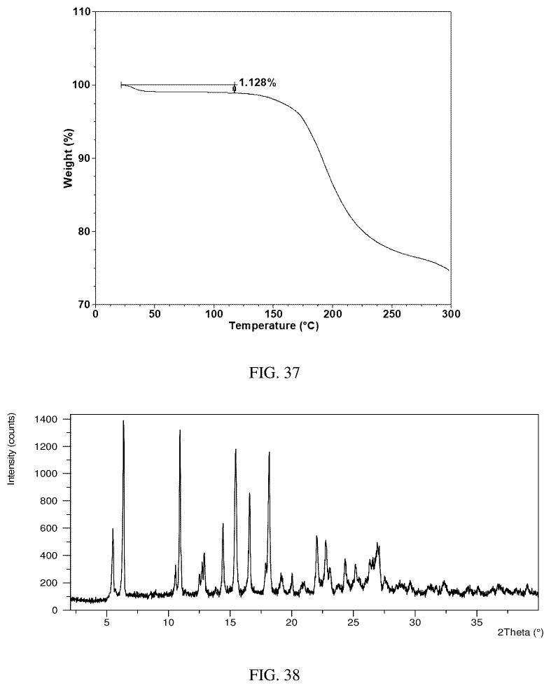

In some embodiments, provided herein is a crystalline Formula I fumarate Form II (Formula I fumarate Form II), wherein the crystalline structure exhibits an X-Ray powder diffraction (XRPD) pattern substantially as shown in FIG. 35. Formula I fumarate Form II may exhibit a differential scanning calorimetry (DSC) thermogram substantially as shown in FIG. 36. Crystalline Formula I fumarate Form II has a thermogravimetric analysis (TGA) thermogram substantially as shown in FIG. 37.

In some embodiments of crystalline Formula I fumarate Form II, at least one, at least two, or all of the following (a)-(c) apply: (a) a crystalline Formula I fumarate Form II has an XRPD pattern substantially as shown in FIG. 35; (b) a crystalline Formula I fumarate Form II has a DSC thermogram substantially as shown in FIG. 36; (c) crystalline Formula I fumarate Form II has a TGA thermogram substantially as shown in FIG. 37.

In some embodiments, crystalline Formula I fumarate Form II has an XRPD pattern displaying at least two, at least three, at least four, at least five, at least six, at least seven, at least eight, or at least nine of the degree 2θ-reflections with the greatest intensity as the XRPD pattern substantially as shown in FIG. 35.

In some embodiments, Formula I fumarate Form II has an XRPD pattern comprising degree 2θ-reflections (±0.2 degrees 2θ) at 5.6, 6.1, and 16.5 degrees.

In some embodiments, Formula I fumarate Form II has an XRPD pattern comprising degree 2θ-reflections (±0.2 degrees 2θ) at 13.6, 16.2, and 24.7 degrees and one, two, or three of the degree 2θ-reflections (±0.2 degrees 2θ) at 5.6, 6.1, and 16.5 degrees.

In some embodiments, Formula I fumarate Form II has an XRPD pattern comprising degree 2θ-reflections (±0.2 degrees 2θ) at 15.6, 18.4, and 25.2 degrees and one, two or three of the degree 2θ-reflections (±0.2 degrees 2θ) at 5.6, 6.1, and 16.5 degrees.

In some embodiments, Formula I fumarate Form II has an XRPD pattern comprising degree 2θ-reflections (±0.2 degrees 2θ) at 5.6, 6.1, 16.5, 13.6, 16.2, 24.7, 15.6, 18.4, and 25.2 degrees.

In some embodiments, Formula I fumarate Form II has a differential scanning calorimetry thermogram having an endothermic transition with an onset at about 25 °C. In some embodiments, Formula I fumarate Form II has a differential scanning calorimetry thermogram having an endothermic transition with an onset at about 162 °C. In some embodiments, Formula I fumarate Form II has a differential scanning calorimetry thermogram having an endothermic transition with an onset at about 25 °C, and a second endothermic transition with an onset at about 162 °C.

### Formula I Fumarate Form III

In some embodiments, provided herein is a crystalline Formula I fumarate Form III (Formula I fumarate Form III), wherein the crystalline structure exhibits an X-Ray powder diffraction (XRPD) pattern substantially as shown in FIG. 38.

In some embodiments, crystalline Formula I fumarate Form III has an XRPD pattern displaying at least two, at least three, at least four, at least five, at least six, at least seven, at least eight, or at least nine of the degree 2θ-reflections with the greatest intensity as the XRPD pattern substantially as shown in FIG. 38.

In some embodiments, Formula I fumarate Form III has an XRPD pattern comprising degree 2θ-reflections (±0.2 degrees 2θ) at 6.4, 10.9, and 15.5 degrees.

In some embodiments, Formula I fumarate Form III has an XRPD pattern comprising degree 2θ-reflections (±0.2 degrees 2θ) at 5.5, 14.4, and 18.2 degrees and one, two, or three of the degree 2θ-reflections (±0.2 degrees 2θ) at 6.4, 10.9, and 15.5 degrees.

In some embodiments, Formula I fumarate Form III has an XRPD pattern comprising degree 2θ-reflections (±0.2 degrees 2θ) at 10.6, 16.6, and 22.0 degrees and one, two or three of the degree 2θ-reflections (±0.2 degrees 2θ) at 6.4, 10.9, and 15.5 degrees.

In some embodiments, Formula I fumarate Form III has an XRPD pattern comprising degree 2θ-reflections (±0.2 degrees 2θ) at 6.4, 13.9, 15.5, 5.5, 14.4, 18.2, 10.6, 16.6, and 22.0 degrees.

Formula I bis-Malonate

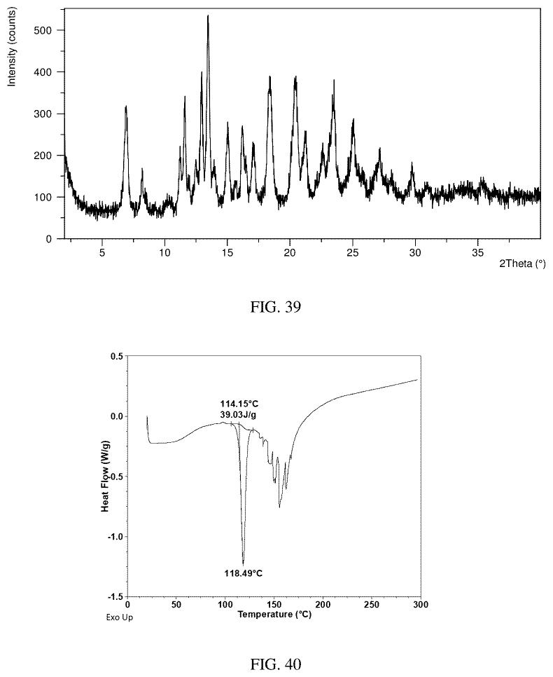

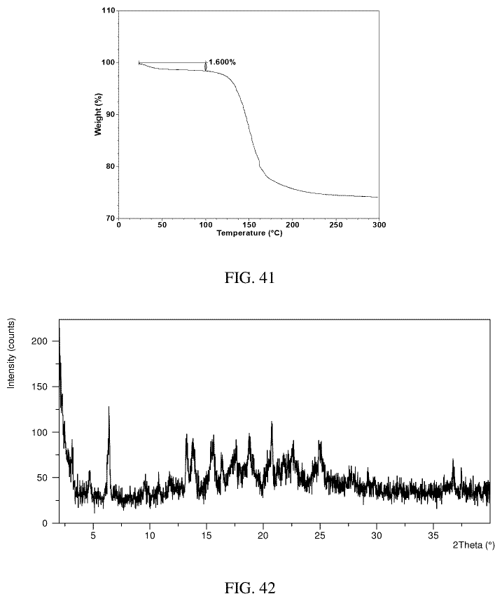

In some embodiments, provided herein is a crystalline Formula I bis-malonate (Formula I bis-malonate), wherein the crystalline structure exhibits an X-Ray powder diffraction (XRPD) pattern substantially as shown in FIG. 39. Formula I bis-malonate may exhibit a differential scanning calorimetry (DSC) thermogram substantially as shown in FIG. 40. Crystalline Formula I bis-malonate has a thermogravimetric analysis (TGA) thermogram substantially as shown in FIG. 41.

In some embodiments of crystalline Formula I bis-malonate, at least one, at least two, or all of the following (a)-(c) apply: (a) a crystalline Formula I bis-malonate has an XRPD pattern substantially as shown in FIG. 39; (b) a crystalline Formula I bis-malonate has a DSC thermogram substantially as shown in FIG. 40; (c) crystalline Formula I bis-malonate has a TGA thermogram substantially as shown in FIG. 41.

In some embodiments, crystalline Formula I bis-malonate has an XRPD pattern displaying at least two, at least three, at least four, at least five, at least six, at least seven, at least eight, or at least nine of the degree 2θ-reflections with the greatest intensity as the XRPD pattern substantially as shown in FIG. 39.

In some embodiments, Formula I bis-malonate has an XRPD pattern comprising degree 2θ-reflections (±0.2 degrees 2θ) at 6.9, 13.5, and 20.4 degrees.

In some embodiments, Formula I bis-malonate has an XRPD pattern comprising degree 2θ-reflections (±0.2 degrees 2θ) at 11.6, 18.4, and 23.6 degrees and one, two, or three of the degree 2θ-reflections (±0.2 degrees 2θ) at 6.9, 13.5, and 20.4 degrees.

In some embodiments, Formula I bis-malonate has an XRPD pattern comprising degree 2θ-reflections (±0.2 degrees 2θ) at 11.2, 12.9, and 16.2 degrees and one, two or three of the degree 2θ-reflections (±0.2 degrees 2θ) at 6.9, 13.5, and 20.4 degrees.

In some embodiments, Formula I bis-malonate has an XRPD pattern comprising degree 2θ-reflections (±0.2 degrees 2θ) at 6.9, 13.5, 20.4, 11.6, 18.4, 23.6, 11.2, 12.9, and 16.2 degrees.

In some embodiments, Formula I bis-malonate has a differential scanning calorimetry thermogram having an endothermic transition with an onset at about 114 °C.

### Formula I L-Tartrate

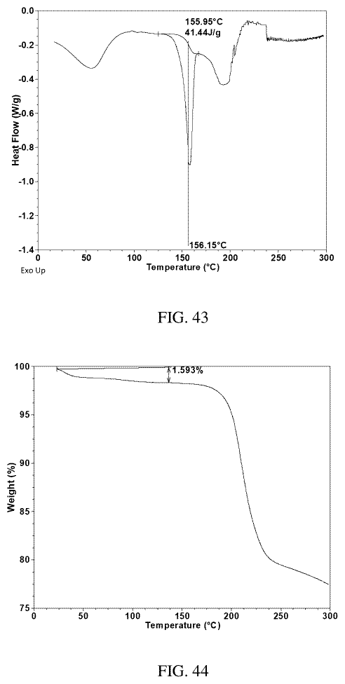

In some embodiments, provided herein is a crystalline Formula I L-tartrate (Formula I L-tartrate), wherein the crystalline structure exhibits an X-Ray powder diffraction (XRPD) pattern substantially as shown in FIG. 42. Formula I L-tartrate may exhibit a differential scanning calorimetry (DSC) thermogram substantially as shown in FIG. 43. Crystalline Formula I L-tartrate has a thermogravimetric analysis (TGA) thermogram substantially as shown in FIG. 44.

In some embodiments of crystalline Formula I L-tartrate, at least one, at least two, or all of the following (a)-(c) apply: (a) a crystalline Formula I L-tartrate has an XRPD pattern substantially as shown in FIG. 42; (b) a crystalline Formula I L-tartrate has a DSC thermogram substantially as shown in FIG. 43; (c) crystalline Formula I L-tartrate has a TGA thermogram substantially as shown in FIG. 44.

In some embodiments, crystalline Formula I L-tartrate has an XRPD pattern displaying at least two, at least three, at least four, at least five, at least six, at least seven, at least eight, or at least nine of the degree 2θ-reflections with the greatest intensity as the XRPD pattern substantially as shown in FIG. 42.

In some embodiments, Formula I L-tartrate has an XRPD pattern comprising degree 2θ-reflections (±0.2 degrees 2θ) at 4.7, 6.4, and 13.8 degrees.

In some embodiments, Formula I L-tartrate has an XRPD pattern comprising degree 2θ-reflections (±0.2 degrees 2θ) at 9.6, 13.2, and 18.8 degrees and one, two, or three of the degree 2θ-reflections (±0.2 degrees 2θ) at 4.7, 6.4, and 13.8 degrees.

In some embodiments, Formula I L-tartrate has an XRPD pattern comprising degree 2θ-reflections (±0.2 degrees 2θ) at 15.6, 16.4, and 17.5 degrees and one, two or three of the degree 2θ-reflections (±0.2 degrees 2θ) at 4.7, 6.4, and 13.8 degrees.

In some embodiments, Formula I L-tartrate has an XRPD pattern comprising degree 2θ-reflections (±0.2 degrees 2θ) at 4.7, 6.4, 13.8, 9.6, 13.2, 18.8, 15.6, 16.4, and 17.5 degrees.

In some embodiments, Formula I L-tartrate has a differential scanning calorimetry thermogram having a broad endothermic transition followed by a second endothermic transitions with an onset at about 156 °C.

### Formula I Mono-Malonate Dichloromethane Solvate

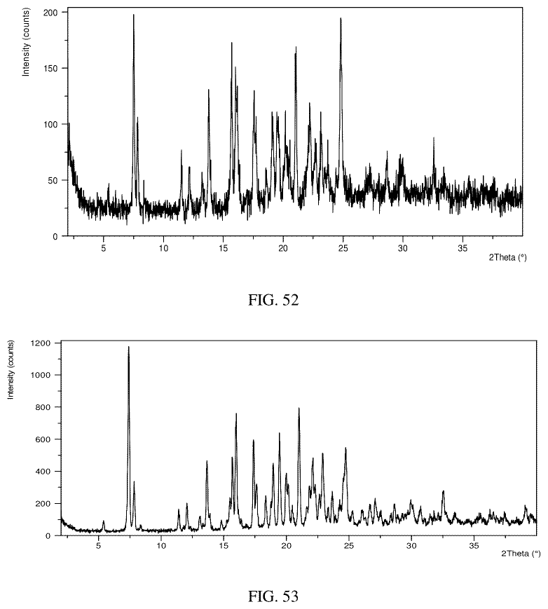

In some embodiments, provided herein is a crystalline Formula I mono-malonate dichloromethane solvate (Formula I mono-malonate dichloromethane solvate), wherein the crystalline structure exhibits an X-Ray powder diffraction (XRPD) pattern substantially as shown in FIG. 52.

In some embodiments, crystalline Formula I mono-malonate dichloromethane solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 52.

In some embodiments, Formula I mono-malonate dichloromethane solvate has an XRPD pattern comprising degree 2θ-reflections (±0.2 degrees 2θ) at 7.5, 13.8, and 15.7 degrees.

In some embodiments, Formula I mono-malonate dichloromethane solvate has an XRPD pattern comprising degree 2θ-reflections (±0.2 degrees 2θ) at 7.8, 11.5, and 16.0 degrees and one, two, or three of the degree 2θ-reflections (±0.2 degrees 2θ) at 7.5, 13.8, and 15.7 degrees.

In some embodiments, Formula I mono-malonate dichloromethane solvate has an XRPD pattern comprising degree 2θ-reflections (±0.2 degrees 2θ) at 12.2, 17.5, and 21.0 degrees and one, two or three of the degree 2θ-reflections (±0.2 degrees 2θ) at 7.5, 13.8, and 15.7 degrees.

In some embodiments, Formula I mono-malonate dichloromethane solvate has an XRPD pattern comprising degree 2θ-reflections (±0.2 degrees 2θ) at 7.5, 13.8, 15.7, 7.8, 11.5, 16.0, 12.2, 17.5, and 21.0 degrees.

### Formula I Mono-Malonate Methyl Ethyl Ketone Solvate

In some embodiments, provided herein is a crystalline Formula I mono-malonate methyl ethyl ketone solvate (Formula I mono-malonate methyl ethyl ketone solvate), wherein the crystalline structure exhibits an X-Ray powder diffraction (XRPD) pattern substantially as shown in FIG. 53.

In some embodiments, crystalline Formula I mono-malonate methyl ethyl ketone solvate has an XRPD pattern displaying at least two, at least three, at least four, at least five, at least six, at least seven, at least eight, or at least nine of the degree 2θ-reflections with the greatest intensity as the XRPD pattern substantially as shown in FIG. 53.

In some embodiments, Formula I mono-malonate methyl ethyl ketone solvate has an XRPD pattern comprising degree 2θ-reflections (±0.2 degrees 2θ) at 7.4, 13.6, and 16.0 degrees.

In some embodiments, Formula I mono-malonate methyl ethyl ketone solvate has an XRPD pattern comprising degree 2θ-reflections (±0.2 degrees 2θ) at 7.9, 12.1, and 15.7 degrees and one, two, or three of the degree 2θ-reflections (±0.2 degrees 2θ) at 7.4, 13.6, and 16.0 degrees.

In some embodiments, Formula I mono-malonate methyl ethyl ketone solvate has an XRPD pattern comprising degree 2θ-reflections (±0.2 degrees 2θ) at 11.4, 17.4, and 19.4 degrees and one, two or three of the degree 2θ-reflections (±0.2 degrees 2θ) at 7.4, 13.6, and 16.0 degrees.

In some embodiments, Formula I mono-malonate methyl ethyl ketone solvate has an XRPD pattern comprising degree 2θ-reflections (±0.2 degrees 2θ) at 7.4, 13.6, 16.0, 7.9, 12.1, 15.7, 11.4, 17.4, and 19.4 degrees.

### Forms of Formula II

Solid forms of Formula II, including crystalline forms and substantially pure forms, maybe suitable for use and an intermediate in the synthesis of an active ingredient, such as for example compounds of Formula's I, III and IV. Thus, solid forms of the compound of Formula II, including crystalline forms and substantially pure forms, may provide the advantage such as improving: the manufacturing process of compounds of Formula I, III, and IV, yield and/or process control.

The use of certain solvents and/or processes have been found to produce different solid forms of the compound of Formula II described herein which may exhibit one or more favorable characteristics described above. The process for the preparation of the solid forms described herein and characterization of these solid forms are described in detail below.

In particular embodiments, novel solid forms, such as crystalline forms of Formula II are disclosed. In some embodiments, a solid form of Formula II freebase Form II is disclosed. In some embodiments, a solid form of Formula II freebase Form II is disclosed. In some embodiments, a solid form of Formula II freebase Form III is disclosed. In some embodiments, a solid form of Formula II freebase Form IV is disclosed. In some embodiments, a solid form of Formula II freebase Form V is disclosed. In some embodiments, a solid form of Formula II freebase Form VI is disclosed. In some embodiments, a solid form of Formula II freebase Form VII is disclosed. In some embodiments, a solid form of Formula II freebase Form VIII is disclosed. In some embodiments, a solid form of Formula II freebase Form IX is disclosed. In some embodiments, a solid form of Formula II freebase Form X is disclosed.

### Formula II Freebase Form I

In some embodiments, provided herein is a crystalline Formula II freebase Form I (Formula II freebase Form I), wherein the crystalline structure exhibits an X-Ray powder diffraction (XRPD) pattern substantially as shown in FIG. 45.

In some embodiments, crystalline Formula II freebase Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 45.

In some embodiments, Formula II freebase Form I has an XRPD pattern comprising degree 2θ-reflections (±0.2 degrees 2θ) at 7.2, 9.9, and 12.5 degrees.

In some embodiments, Formula II freebase Form I has an XRPD pattern comprising degree 2θ-reflections (±0.2 degrees 2θ) at 6.8, 8.0, and 15.3 degrees and one, two, or three of the degree 2θ-reflections (±0.2 degrees 2θ) at 7.2, 9.9, and 12.5 degrees.

In some embodiments, Formula II freebase Form I has an XRPD pattern comprising degree 2θ-reflections (±0.2 degrees 2θ) at 14.7, 17.6, and 19.8 degrees and one, two or three of the degree 2θ-reflections (±0.2 degrees 2θ) at 7.2, 9.9, and 12.5 degrees.

In some embodiments, Formula II freebase Form I has an XRPD pattern comprising degree 2θ-reflections (±0.2 degrees 2θ) at 7.2, 9.9, 12.5, 6.8, 8.0, 15.3, 14.7, 17.6, and 19.8 degrees.

### Formula II Freebase Form II

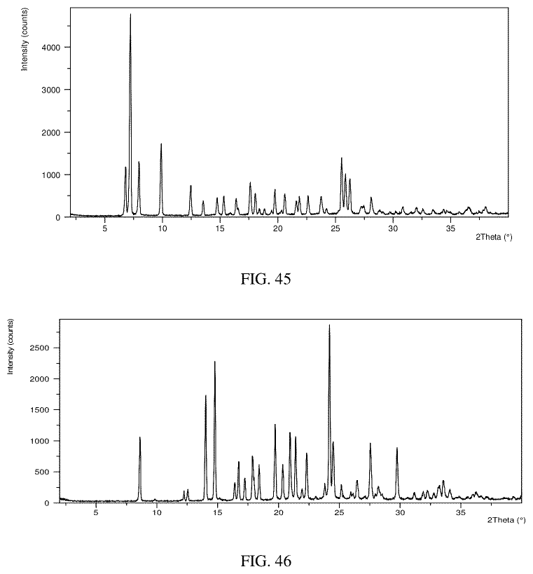

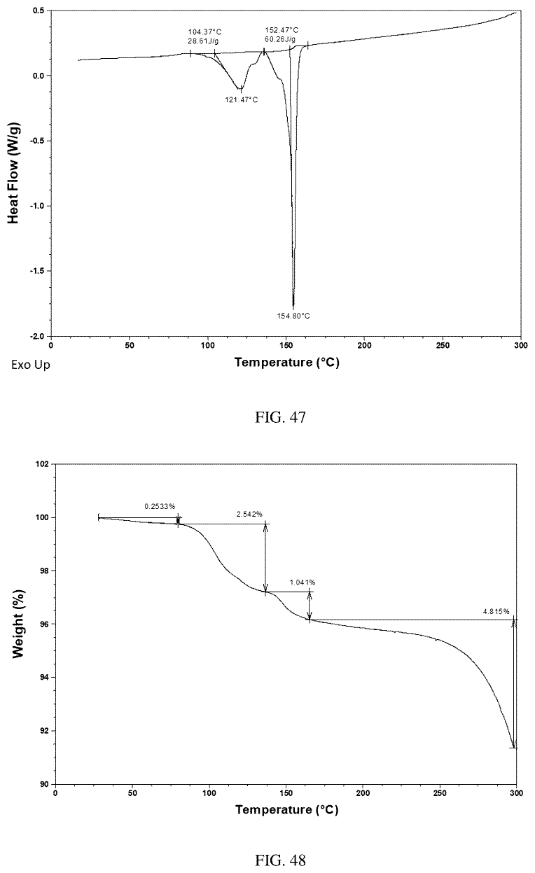

In some embodiments, provided herein is a crystalline Formula II freebase Form II (Formula II freebase Form II), wherein the crystalline structure exhibits an X-Ray powder diffraction (XRPD) pattern substantially as shown in FIG. 46. Formula II freebase Form II may exhibit a differential scanning calorimetry (DSC) thermogram substantially as shown in FIG. 47. Crystalline Formula II freebase Form II has a thermogravimetric analysis (TGA) thermogram substantially as shown in FIG. 48.

In some embodiments of crystalline Formula II freebase Form II, at least one, at least two, or all of the following (a)-(c) apply: (a) a crystalline Formula II freebase Form II has an XRPD pattern substantially as shown in FIG. 46; (b) a crystalline Formula II freebase Form II has a DSC thermogram substantially as shown in FIG. 47; (c) crystalline Formula II freebase Form II has a TGA thermogram substantially as shown in FIG. 48.

In some embodiments, crystalline Formula II freebase Form II has an XRPD pattern displaying at least two, at least three, at least four, at least five, at least six, at least seven, or at least eight, of the degree 2θ-reflections with the greatest intensity as the XRPD pattern substantially as shown in FIG. 46.

In some embodiments, Formula II freebase Form II has an XRPD pattern comprising degree 2θ-reflections (±0.2 degrees 2θ) at 8.6, 14.0, and 16.7 degrees.

In some embodiments, Formula II freebase Form II has an XRPD pattern comprising degree 2θ-reflections (±0.2 degrees 2θ) at 12.6, 14.8, and 17.8 degrees and one, two, or three of the degree 2θ-reflections (±0.2 degrees 2θ) at 8.6, 14.0, and 16.7 degrees.

In some embodiments, Formula II freebase Form II has an XRPD pattern comprising degree 2θ-reflections (±0.2 degrees 2θ) at 16.4, 18.4, 19.7 degrees and one, two or three of the degree 2θ-reflections (±0.2 degrees 2θ) at 8.6, 14.0, and 16.7 degrees.

In some embodiments, Formula II freebase Form II has an XRPD pattern comprising degree 2θ-reflections (±0.2 degrees 2θ) at 8.6, 14.0, 16.7, 12.6, 14.8, 17.8, 16.4, 18.4, and 19.7 degrees.

In some embodiments, Formula II freebase Form II has a differential scanning calorimetry thermogram having an endothermic transition with an onset at about 104 °C. In some embodiments, Formula II freebase Form II has a differential scanning calorimetry thermogram having an endothermic transition with an onset at about 152 °C. In some embodiments, Formula II freebase Form II has a differential scanning calorimetry thermogram having an endothermic transition with an onset at about 104 °C, and a second endothermic transition with an onset at about 152 °C.

### Formula II Freebase Form III

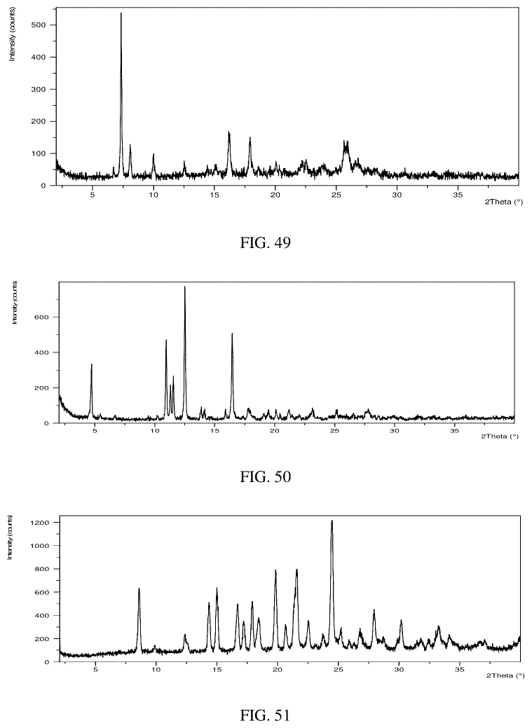

In some embodiments, provided herein is a crystalline Formula II freebase Form III (Formula II freebase Form III), wherein the crystalline structure exhibits an X-Ray powder diffraction (XRPD) pattern substantially as shown in FIG. 49.

In some embodiments, crystalline Formula II freebase Form III has an XRPD pattern displaying at least two, at least three, at least four, at least five, at least six, at least seven, at least eight, or at least nine of the degree 2θ-reflections with the greatest intensity as the XRPD pattern substantially as shown in FIG. 49.

In some embodiments, Formula II freebase Form III has an XRPD pattern comprising degree 2θ-reflections (±0.2 degrees 2θ) at 7.3, 10.0, and 16.2 degrees.

In some embodiments, Formula II freebase Form III has an XRPD pattern comprising degree 2θ-reflections (±0.2 degrees 2θ) at 8.1, 12.5, and 17.9 degrees and one, two, or three of the degree 2θ-reflections (±0.2 degrees 2θ) at 7.3, 10.0, and 16.2 degrees.

In some embodiments, Formula II freebase Form III has an XRPD pattern comprising degree 2θ-reflections (±0.2 degrees 2θ) at 6.7, 15.2, and 25.9 degrees and one, two or three of the degree 2θ-reflections (±0.2 degrees 2θ) at 7.3, 10.0, and 16.2 degrees.

In some embodiments, Formula II freebase Form III has an XRPD pattern comprising degree 2θ-reflections (±0.2 degrees 2θ) at 7.3, 10.0, 16.2, 8.1, 12.5, 17.9, 6.7, 15.2, and 25.9 degrees.

### Formula II Freebase Form IV

In some embodiments, provided herein is a crystalline Formula II freebase Form IV (Formula II freebase Form IV), wherein the crystalline structure exhibits an X-Ray powder diffraction (XRPD) pattern substantially as shown in FIG. 50.

In some embodiments, crystalline Formula II freebase Form IV has an XRPD pattern displaying at least two, at least three, at least four, at least five, at least six, at least seven, at least eight, or at least nine of the degree 2θ-reflections with the greatest intensity as the XRPD pattern substantially as shown in FIG. 50.

In some embodiments, Formula II freebase Form IV has an XRPD pattern comprising degree 2θ-reflections (±0.2 degrees 2θ) at 4.7, 11.0, and 12.5 degrees.

In some embodiments, Formula II freebase Form IV has an XRPD pattern comprising degree 2θ-reflections (±0.2 degrees 2θ) at 11.3, 11.5, and 16.4 degrees and one, two, or three of the degree 2θ-reflections (±0.2 degrees 2θ) at 4.7, 11.0, and 12.5 degrees.

In some embodiments, Formula II freebase Form IV has an XRPD pattern comprising degree 2θ-reflections (±0.2 degrees 2θ) at 13.9, 14.1, and 17.8 degrees and one, two or three of the degree 2θ-reflections (±0.2 degrees 2θ) at 4.7, 11.0, and 12.5 degrees.

In some embodiments, Formula II freebase Form IV has an XRPD pattern comprising degree 2θ-reflections (±0.2 degrees 2θ) at 4.7, 11.0, 12.5, 11.3, 11.5, 16.4, 13.9, 14.1, and 17.8 degrees.

In some embodiments, Formula II freebase Form VI has a differential scanning calorimetry thermogram having an endothermic transition with an onset at about 151.8 °C.

### Formula H Freebase Form V

In some embodiments, provided herein is a crystalline Formula II freebase Form V (Formula II freebase Form V), wherein the crystalline structure exhibits an X-Ray powder diffraction (XRPD) pattern substantially as shown in FIG. 51.

In some embodiments, crystalline Formula II freebase Form V has an XRPD pattern displaying at least two, at least three, at least four, at least five, at least six, at least seven, at least eight, or at least nine of the degree 2θ-reflections with the greatest intensity as the XRPD pattern substantially as shown in FIG. 51.

In some embodiments, Formula II freebase Form V has an XRPD pattern comprising degree 2θ-reflections (±0.2 degrees 2θ) at 8.6, 12.4, and 15.0 degrees.

In some embodiments, Formula II freebase Form V has an XRPD pattern comprising degree 2θ-reflections (±0.2 degrees 2θ) at 14.3, 16.7, and 17.9 degrees and one, two, or three of the degree 2θ-reflections (±0.2 degrees 2θ) at 8.6, 12.4, and 15.0 degrees.

In some embodiments, Formula II freebase Form V has an XRPD pattern comprising degree 2θ-reflections (±0.2 degrees 2θ) at 17.2, 18.5, and 19.8 degrees and one, two or three of the degree 2θ-reflections (±0.2 degrees 2θ) at 8.6, 12.4, and 15.0 degrees.

In some embodiments, Formula II freebase Form V has an XRPD pattern comprising degree 2θ-reflections (±0.2 degrees 2θ) at 8.6, 12.4, 15.0, 14.3, 16.7, 17.9, 17.2, 18.5, and 19.8 degrees.

### Formula II Freebase Form VI

In some embodiments, provided herein is a crystalline Formula II freebase Form VI (Formula II freebase Form VI), wherein the crystalline structure exhibits an X-Ray powder diffraction (XRPD) pattern substantially as shown in FIG. 57.

In some embodiments, crystalline Formula II freebase Form VI has an XRPD pattern displaying at least two, at least three, at least four, at least five, at least six, at least seven, at least eight, or at least nine of the degree 2θ-reflections with the greatest intensity as the XRPD pattern substantially as shown in FIG. 57.

In some embodiments, Formula II freebase Form VI has an XRPD pattern comprising degree 2θ-reflections (±0.2 degrees 2θ) at 5.8, 12.8, and 7.8 degrees.

In some embodiments, Formula II freebase Form VI has an XRPD pattern comprising degree 2θ-reflections (±0.2 degrees 2θ) at 12.0, 18.4, and 24.2 degrees and one, two, or three of the degree 2θ-reflections (±0.2 degrees 2θ) at 5.8, 12.8, and 7.8 degrees.

In some embodiments, Formula II freebase Form VI has an XRPD pattern comprising degree 2θ-reflections (±0.2 degrees 2θ) at 11.4, 15.5, and 19.0 degrees and one, two or three of the degree 2θ-reflections (±0.2 degrees 2θ) at 5.8, 12.8, and 7.8 degrees.

In some embodiments, Formula II freebase Form VI has an XRPD pattern comprising degree 2θ-reflections (±0.2 degrees 2θ) at 5.8. 12.8, 7.8, 12.0, 18.4, 24.2, 11.4, 15.5, and 19.0 degrees.

In some embodiments, Formula II freebase Form VI has a differential scanning calorimetry thermogram having an exothermic transition with an onset at about 88.7 °C. In some embodiments, Formula II freebase Form VI has a differential scanning calorimetry thermogram having an endothermic transition with an onset at about 146.1 °C. In some embodiments, Formula II freebase Form VI has a differential scanning calorimetry thermogram having an exothermic transition with an onset at about 88.7 °C and an endothermic transition with an onset at about 146.1 °C.

### Formula II Freebase Form VII

In some embodiments, provided herein is a crystalline Formula II freebase Form VII (Formula II freebase Form VII), wherein the crystalline structure exhibits an X-Ray powder diffraction (XRPD) pattern substantially as shown in FIG. 59.

In some embodiments, crystalline Formula II freebase Form VII has an XRPD pattern displaying at least two, at least three, at least four, at least five, at least six, at least seven, at least eight, or at least nine of the degree 2θ-reflections with the greatest intensity as the XRPD pattern substantially as shown in FIG. 59.

In some embodiments, Formula II freebase Form VII has an XRPD pattern comprising degree 2θ-reflections (±0.2 degrees 2θ) at 8.6, 10.8, and 17.2 degrees.

In some embodiments, Formula II freebase Form VII has an XRPD pattern comprising degree 2θ-reflections (±0.2 degrees 2θ) at 8.0, 12.0, and 15.7 degrees and one, two, or three of the degree 2θ-reflections (±0.2 degrees 2θ) at 8.6, 10.8, and 17.2 degrees.

In some embodiments, Formula II freebase Form VII has an XRPD pattern comprising degree 2θ-reflections (±0.2 degrees 2θ) at 15.2, 20.2, and 24.2 degrees and one, two or three of the degree 2θ-reflections (±0.2 degrees 2θ) at 8.6, 10.8, and 17.2 degrees.

In some embodiments, Formula II freebase Form VII has an XRPD pattern comprising degree 2θ-reflections (±0.2 degrees 2θ) at 8.6, 10.8, 17.2, 8.0, 12.0, 15.7, 15.2, 20.2, and 24.2 degrees.

### Formula II Freebase Form VIII

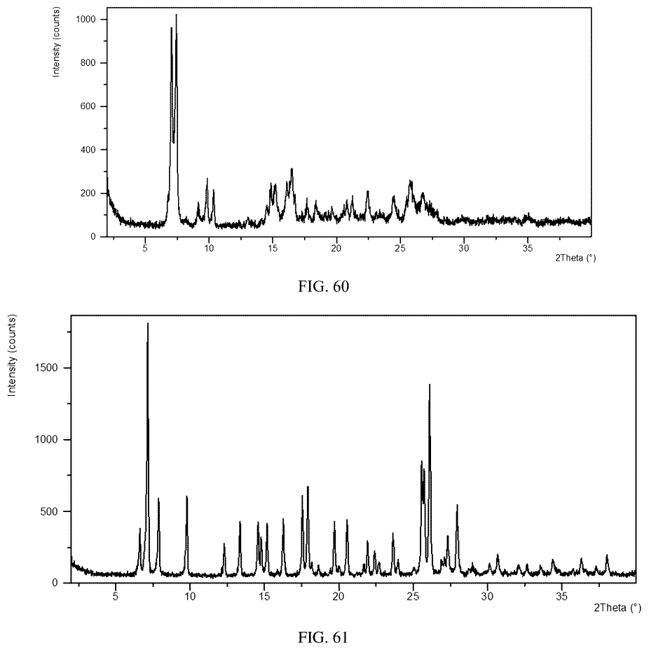

In some embodiments, provided herein is a crystalline Formula II freebase Form VIII (Formula II freebase Form VIII), wherein the crystalline structure exhibits an X-Ray powder diffraction (XRPD) pattern substantially as shown in FIG. 60.

In some embodiments, crystalline Formula II freebase Form VIII has an XRPD pattern displaying at least two, at least three, at least four, at least five, at least six, at least seven, at least eight, or at least nine of the degree 2θ-reflections with the greatest intensity as the XRPD pattern substantially as shown in FIG. 60.

In some embodiments, Formula II freebase Form VIII has an XRPD pattern comprising degree 2θ-reflections (±0.2 degrees 2θ) at 7.4, 9.9, and 16.5 degrees.

In some embodiments, Formula II freebase Form VIII has an XRPD pattern comprising degree 2θ-reflections (±0.2 degrees 2θ) at 7.0, 10.4, and 22.5 degrees and one, two, or three of the degree 2θ-reflections (±0.2 degrees 2θ) at 7.4, 9.9, and 16.5 degrees.

In some embodiments, Formula II freebase Form VIII has an XRPD pattern comprising degree 2θ-reflections (±0.2 degrees 2θ) at 9.1, 15.2, and 24.4 degrees and one, two or three of the degree 2θ-reflections (±0.2 degrees 2θ) at 7.4, 9.9, and 16.5 degrees.

In some embodiments, Formula II freebase Form VIII has an XRPD pattern comprising degree 2θ-reflections (±0.2 degrees 2θ) at 7.4, 9.9, 16.5, 7.0, 10.4, 22.5, 9.1, 15.2, and 24.4 degrees.

### Formula II Freebase Form IX

In some embodiments, provided herein is a crystalline Formula II freebase Form IX (Formula II freebase Form IX), wherein the crystalline structure exhibits an X-Ray powder diffraction (XRPD) pattern substantially as shown in FIG. 61.

In some embodiments, crystalline Formula II freebase Form IX has an XRPD pattern displaying at least two, at least three, at least four, at least five, at least six, at least seven, at least eight, or at least nine of the degree 2θ-reflections with the greatest intensity as the XRPD pattern substantially as shown in FIG. 61.

In some embodiments, Formula II freebase Form IX has an XRPD pattern comprising degree 2θ-reflections (±0.2 degrees 2θ) at 7.2, 9.8, and 13.4 degrees.

In some embodiments, Formula II freebase Form IX has an XRPD pattern comprising degree 2θ-reflections (±0.2 degrees 2θ) at 7.9, 15.2, and 17.9 degrees and one, two, or three of the degree 2θ-reflections (±0.2 degrees 2θ) at 7.2, 9.8, and 13.4 degrees.

In some embodiments, Formula II freebase Form IX has an XRPD pattern comprising degree 2θ-reflections (±0.2 degrees 2θ) at 6.6, 12.3, and 16.3 degrees and one, two or three of the degree 2θ-reflections (±0.2 degrees 2θ) at 7.2, 9.8, and 13.4 degrees.

In some embodiments, Formula II freebase Form IX has an XRPD pattern comprising degree 2θ-reflections (±0.2 degrees 2θ) at 7.2, 9.8, 13.4, 7.9, 15.2, 17.9, 6.6, 12.3, and 16.3 degrees.

### Formula II Freebase Form X

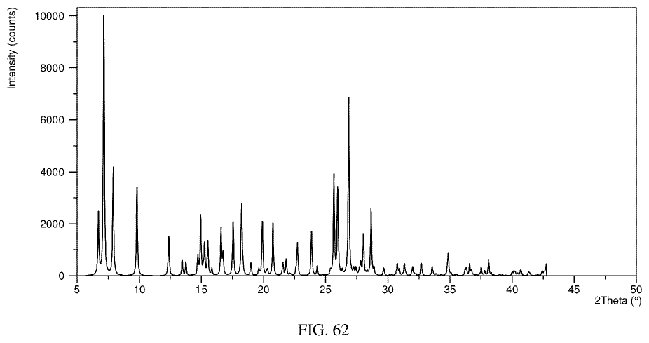

In some embodiments, provided herein is a crystalline Formula II freebase Form X (Formula II freebase Form X), wherein the crystalline structure exhibits an X-Ray powder diffraction (XRPD) pattern substantially as shown in FIG. 62.

In some embodiments, crystalline Formula II freebase Form X has an XRPD pattern displaying at least two, at least three, at least four, at least five, at least six, at least seven, at least eight, or at least nine of the degree 2θ-reflections with the greatest intensity as the XRPD pattern substantially as shown in FIG. 62.

In some embodiments, Formula II freebase Form X has an XRPD pattern comprising degree 2θ-reflections (±0.2 degrees 2θ) at 7.2, 9.8, and 12.4 degrees.

In some embodiments, Formula II freebase Form X has an XRPD pattern comprising degree 2θ-reflections (±0.2 degrees 2θ) at 7.9, 17.6, and 20.8 degrees and one, two, or three of the degree 2θ-reflections (±0.2 degrees 2θ) at 7.2, 9.8, and 12.4 degrees.

In some embodiments, Formula II freebase Form X has an XRPD pattern comprising degree 2θ-reflections (±0.2 degrees 2θ) at 6.7, 18.2, and 26.8 degrees and one, two or three of the degree 2θ-reflections (±0.2 degrees 2θ) at 7.2, 9.8, and 12.4 degrees.

In some embodiments, Formula II freebase Form X has an XRPD pattern comprising degree 2θ-reflections (±0.2 degrees 2θ) at 7.2, 9.8, 12.4, 7.9, 17.6, 20.8, 6.7, 18.2, and 26.8 degrees.

### Formula III and IV

Further provided herein are compounds that function as modulators of α4β7 integrin. In some embodiments, the present disclosure provides for a compound of Formula III, or a pharmaceutically acceptable salt thereof:

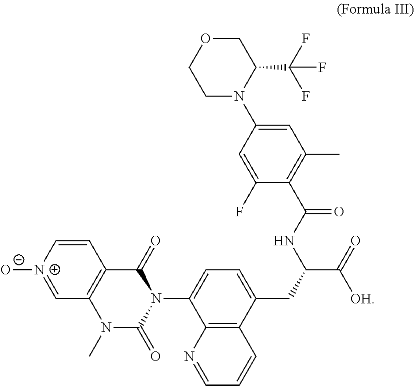

In some embodiments, the present disclosure provides for a compound of Formula IV, or a pharmaceutically acceptable salt thereof:

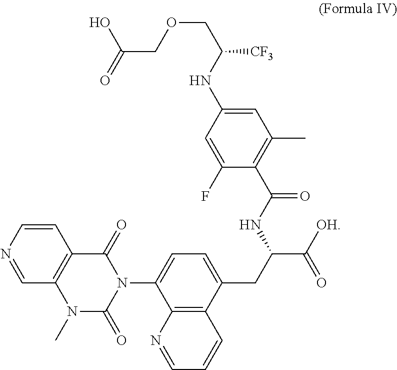 [SMILES: Cc1cc(N[C@H](COCC(=O)O)C(F)(F)F)cc(F)c1C(=O)N[C@@H](Cc1ccc(-n2c(=O)c3ccncc3n(C)c2=O)c2ncccc12)C(=O)O]

In some embodiments, the present disclosure provides compounds which modulate α4β7 integrin. In some embodiments the compounds, or pharmaceutically acceptable salt thereof which is selected from:

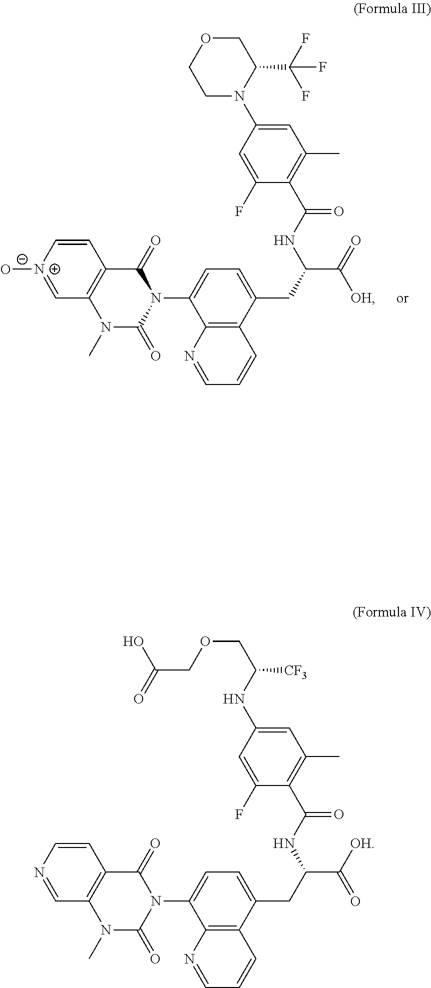

Also, provided are compounds Formula III or Formula IV or pharmaceutically acceptable salts, isomer, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

In some embodiments, compounds of Formula III and Formula IV may be optical isomers, racemates, or other mixtures thereof of the compounds described herein or pharmaceutically acceptable salts or a mixture thereof. In those situations, the single enantiomer or diastereomer, i.e., optically active form, can be obtained by asymmetric synthesis or by resolution of the racemate. Resolution of racemates can be accomplished, for example, by conventional methods such as crystallization in the presence of a resolving agent, or chromatography, using, for example a chiral high pressure liquid chromatography (HPLC) column. In addition, provided are also Z- and E-forms (or cis- and trans-forms) of the hydroxyamidine compounds described herein. Specifically, Z- and E-forms are included even if only one designation is named for both carbon-carbon double bonds as well as the hydroxyamidine bond.

Where chirality is not specified but is present, it is understood that the embodiment is directed to either the specific diastereomerically or enantiomerically enriched form; or a racemic or scalemic mixture of such compound(s).

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

“Atropisomers” are stereoisomers arising due to hindered rotation about a single bond, where the barrier to rotation about the bond is high enough to allow for isolation of individual stereoisomers.

In certain embodiments, provided are atropisomers thereof of the compounds described herein or pharmaceutically acceptable salts.

### Pharmaceutical Compositions

For the purposes of administration, in certain embodiments, the compounds described herein are administered as a raw chemical or are formulated as pharmaceutical compositions. Pharmaceutical compositions of the present disclosure can include a therapeutically effective amount of a compound of Formula I, III, or IV and at least one pharmaceutically acceptable carrier and/or excipient. The compound of Formula I, III, or IV is present in the composition in an amount which is effective to treat a particular disease or condition of interest. The pharmaceutical compositions of the present disclosure may additionally comprise one or more other compounds as active ingredients, including for instance prodrugs, other nuclear receptor modulators, or other active pharmaceutical ingredients such as active pharmaceutical ingredients for use in treating liver disease, such as Glucagon-Like Peptide-1 (GLP-1) receptor agonists, Tumor progression locus-2 (Tpl-2) inhibitors, Interleukin-12-23 (IL-12-23) p40 inhibitors, Interleukin-23 (IL-23) inhibitors, and Janus kinase (JAK) inhibitors. In some embodiments, the pharmaceutical compositions of the present disclosure additionally comprise an ACC inhibitor and an ASK1 inhibitor.

In some embodiments, a pharmaceutical composition includes Formula I freebase Form I; Formula I freebase acetophenone solvate; Formula I freebase anisole solvate; Formula I gentisate Form I; Formula I mono-malonate monohydrate acetonitrile solvate; Formula I mono-malonate Form I; Formula I mono-malonate From II; Formula I mono-malonate acetone solvate; Formula I mono-malonate 2-methyltetrahydrofuran solvate; Formula I mono-malonate tetrahydrofuran solvate; Formula I xinafoate Form I; Formula I xinafoate Form II; Formula I oxalate; Formula I fumarate Form I; Formula I fumarate Form II; Formula I fumarate Form III; Formula I bis-malonate; Formula I L-tartrate; Formula III; and/or Formula IV. In some embodiments, a pharmaceutical composition includes Formula I freebase Form I; Formula I freebase acetophenone solvate; Formula I freebase anisole solvate; Formula I gentisate Form I; Formula I mono-malonate monohydrate acetonitrile solvate; Formula I mono-malonate Form I; Formula I mono-malonate From II; Formula I mono-malonate acetone solvate; Formula I mono-malonate 2-methyltetrahydrofuran solvate; Formula I mono-malonate tetrahydrofuran solvate; Formula I xinafoate Form I; Formula I xinafoate Form II; Formula I oxalate; Formula I fumarate Form I; Formula I fumarate Form II; Formula I fumarate Form III; Formula I bis-malonate; Formula I L-tartrate; Formula III; and/or Formula IV and a pharmaceutically acceptable carrier.

The activity of compounds of Formula I, III, or IV can be determined by one skilled in the art, for example, as described herein. Appropriate therapeutically effective concentrations and dosages can be readily determined by one skilled in the art.

In certain embodiments, the crystalline, salt, and/or solvate forms described herein may potentially exhibit improved properties. For example, in certain embodiments, the crystalline and/or salt forms described herein may potentially exhibit improved stability. Such improved stability could have a potentially beneficial impact on the manufacture of the compound of Formula I, such as, for example, offering the ability to store process intermediate for extended periods of time. Improved stability could also potentially benefit a composition or pharmaceutical composition of the compound of Formula I, III, or IV. In certain embodiments, the crystalline salt, and/or solvate forms described herein may also potentially result in improved yield of the compound of Formula I, III, or IV, or in an improvement of the quality of the compound of Formula I, III, or IV. In certain embodiments, the crystalline, salt, and/or solvate forms described herein may also exhibit improved pharmacokinetic properties and/or potentially improved bioavailability.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the present disclosure can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate, microcrystalline cellulose, lactose monohydrate, mannitol or colloidal silicon dioxide; a disintegrating agent such as corn starch, potato starch, alginic acid, croscarmellose sodium or crospovidone; a lubricant such as magnesium stearate; and a sweetening agent such as sucrose, lactose or saccharin. When a dosage unit form is a capsule, it may contain, in addition to materials of the above type, a liquid carrier such as a fatty oil or pharmaceutical media, such as, for example, water, glycols (e.g., polyethylene glycol 400), or alcohols.

Various other materials may be present as coatings or to modify the physical form of the dosage unit. For instance, tablets may be coated with shellac, sugar, polyvinyl alcohol, polyethylene glycol 3350, titanium dioxide, talc, coloring agent, or combinations thereof. A syrup or elixir may contain, in addition to the active ingredient, sucrose as a sweetening agent, methyl and propylparabens as preservatives, a dye and a flavoring such as cherry or orange flavor.

The compounds of the present disclosure may also be administered parenterally. Solutions or suspensions of these active compounds can be prepared in water suitably mixed with an organic, an additive, or combinations thereof. Examples of organics include, but are not limited to, N-methyl pyrrolidone, dimethylsulfoxide, polyethylene glycols, and combinations thereof. Examples of additives include, but are not limited to, hydroxypropyl cellulose, polyvinylpyrrolidone, poloxamers, poly(lactic-co-glycolic acid), polysorbates, povidone, carboxymethylcellulose, and combinations thereof. Dispersions can also be prepared in glycerol, liquid polyethylene glycols and mixtures thereof in oils. Under ordinary conditions of storage and use, these preparations may contain a preservative to, for instance, prevent the growth of microorganisms. In some embodiments, the parenteral administration includes intravenous administration with formulations comprising solutions with a mixture of organics and aqueous media. In some embodiments, the intravenous administration is dosed as a 100% organic solution.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. For instance, the forms can be stable under the conditions of manufacture and storage. The forms can be preserved against the contaminating action of microorganisms such as bacteria and fungi (for instance, via use of preservatives). The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present disclosure. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In some embodiments, compounds of the present disclosure are administered orally.

In particular embodiments of compositions comprising a crystalline form of Formula I or a pharmaceutically acceptable salt thereof, at least about 50%, at least about 60%, at least about 70%, at least about 80%, at least about 85%, at least about 90%, at least about 95%, at least about 96%, at least about 97%, at least about 98%, or at least about 99% of Formula I present in the composition is one of the crystalline forms disclosed herein. In certain embodiments, the composition includes at least about 50%, at least about 60%, at least about 70%, at least about 80%, at least about 85%, at least about 90%, at least about 95%, at least about 96%, at least about 97%, at least about 98%, or at least about 99% of one of the crystalline forms of Formula I.

In other embodiments of compositions comprising a crystalline form disclosed herein, less than about 50%, less than about 40%, less than about 30%, less than about 20%, less than about 10%, less than about 5%, less than about 4%, less than about 3%, less than about 2% or less than about 1% of Formula I present in the composition are other amorphous or crystal forms of Formula I and/or impurities.

In yet other embodiments of compositions comprising the crystalline forms disclosed herein, impurities make up less than about 5%, less than about 4%, less than about 3%, less than about 2% or less than about 1% of the total mass relative to the mass of the crystalline forms present. Impurities may, for example, include by-products from synthesizing Formula I, contaminants, degradation products, other crystalline forms, amorphous form, water, and solvents. In certain embodiments, impurities include by-products from the process of synthesizing Formula I. In certain embodiments, impurities include contaminants from the process of synthesizing Formula I. In certain embodiments, impurities include degradation products of Formula I. In certain embodiments, impurities include other crystalline forms of Formula I. In certain embodiments, impurities include other crystalline forms of Formula I and/or amorphous forms of Formula I. In certain embodiments, impurities include water or solvent. In certain embodiments of compositions comprising a crystalline form disclosed herein, impurities are selected from the group consisting of by-products from synthesizing Formula I, contaminants, degradation products, other crystalline forms, amorphous forms, water, solvents and combinations thereof.

### Dosage

The effective dosage of active ingredient employed may vary depending on the particular compound employed, the mode of administration, the condition being treated and the severity of the condition being treated. Such dosage may be ascertained readily by a person skilled in the art.

When treating or preventing α4β7 integrin mediated conditions for which compounds of the present disclosure are indicated, generally satisfactory results are obtained when the compounds of the present disclosure are administered at a daily dosage of from about 0.1 milligram to about 100 milligram per kilogram of animal body weight. In some embodiments, the compounds of the present disclosure are given as a single daily dose or in divided doses two to six times a day, or in sustained release form. For most large mammals, the total daily dosage can be from about 1 milligram to about 1000 milligrams. In the case of a 70 kg adult human, the total daily dose will generally be from about 7 milligrams to about 350 milligrams. This dosage regimen may be adjusted to provide the optimal therapeutic response. In some embodiments, the total daily dosage is from about 1 milligram to about 900 milligrams, about 10 milligrams to about 800 milligrams, about 20 milligrams to about 700 milligrams, about 30 milligrams to about 600 milligrams, about 40 milligrams to about 550 milligrams, or about 50 milligrams to about 400 milligrams. In some embodiments, the total daily dosage is from about 10 milligrams to about 50 milligrams, from about 20 milligrams to about 40 milligrams, from about 25 milligrams to about 35 milligrams, from about 50 milligrams to about 150 milligrams, from about 70 milligrams to about 130 milligrams, from about 80 milligrams to about 120 milligrams, from about 90 milligrams to about 100 milligrams, from about 1 milligram to about 150 milligrams, from about 1 milligram to about 75 milligrams, from about 1 milligram to about 50 milligrams, from about 25 milligrams to about 125 milligrams, from about 125 milligrams to about 275 milligrams, from about 275 milligrams to about 425 milligrams, from about 425 milligrams to about 575 milligrams, from about 575 milligrams to about 725 milligrams, from about 725 milligrams to about 875 milligrams, or from about 875 milligrams to about 1000 milligrams.

The compounds of the present application or the compositions thereof may be administered once, twice, three, or four times daily, using any suitable mode described above. Also, administration or treatment with the compounds may be continued for a number of days or months; for example, commonly treatment would continue for at least 7 days, 14 days, or 28 days, or 1, 2, 3, 4, 5, 6, 7, 8, 9, 10, 11, or 12 months.

In a particular embodiment, the methods provided herein comprise administering to the subject an initial daily dose of about 1 mg to about 1500 mg of a compound described herein, such as 150 to 600 mg, for example 150 mg, 300 mg, 600 mg. In further embodiments, the methods comprise increasing the dose by increments until clinical efficacy is achieved. Increments of about 5, 10, 25, 30, 40, 50, or 100 mg can be used to increase the dose. The dosage can be increased daily, every other day, twice per week, once per week or once every 4 weeks.

### Treatment Methods and Uses

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The disclosure further relates to the use of said compounds for the treatment and/or prophylaxis of diseases and/or conditions through binding of said nuclear receptor by said compounds. Further the present disclosure relates to the use of said compounds for the preparation of a medicament for the treatment and/or prophylaxis of diseases and/or conditions through binding of said nuclear receptor by said compounds.

Also provided herein are methods of treating a patient having a Cot mediated condition. In some embodiments, the method includes administering a compound or composition disclosed herein. In some embodiments, a method of treating a patient having an Cot mediated condition includes administering a therapeutically effective amount of Formula I freebase Form I; Formula I freebase acetophenone solvate; Formula I freebase anisole solvate; Formula I gentisate Form I; Formula I mono-malonate monohydrate acetonitrile solvate; Formula I mono-malonate Form I; Formula I mono-malonate From II; Formula I mono-malonate acetone solvate; Formula I mono-malonate 2-methyltetrahydrofuran solvate; Formula I mono-malonate tetrahydrofuran solvate; Formula I xinafoate Form I; Formula I xinafoate Form II; Formula I oxalate; Formula I fumarate Form I; Formula I fumarate Form II; Formula I fumarate Form III; Formula I bis-malonate; and/or Formula I L-tartrate.

Also provided herein are methods of treating or preventing a disease or condition in a patient in need thereof, comprising administering a therapeutically effective amount of a compound useful for inhibiting α4β7 integrin, wherein the disease or condition is an inflammatory disease, and wherein the compound useful for inhibiting α4β7 integrin is a compound of Formula I

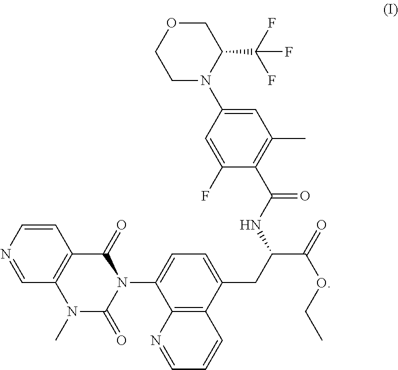

Also provided herein are methods of treating or preventing a disease or condition in a patient in need thereof, comprising administering a therapeutically effective amount of a compound useful for modulating α4β7 integrin, wherein the disease or condition is an inflammatory disease, and wherein the compound useful for modulating α4β7 integrin is a compound of Formula III

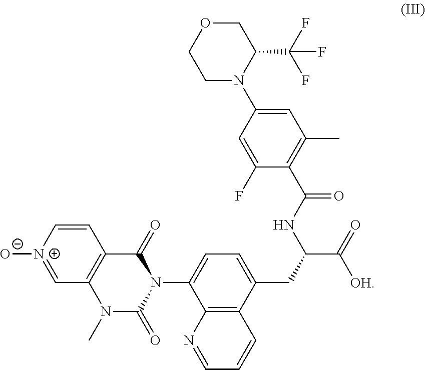

Also provided herein are methods of treating or preventing a disease or condition in a patient in need thereof, comprising administering a therapeutically effective amount of a compound useful for modulating α4β7 integrin, wherein the disease or condition is an inflammatory disease, and wherein the compound useful for modulating α4β7 integrin is a compound of Formula IV

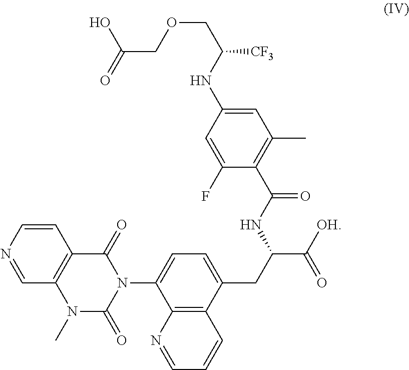 [SMILES: Cc1cc(N[C@H](COCC(=O)O)C(F)(F)F)cc(F)c1C(=O)N[C@@H](Cc1ccc(-n2c(=O)c3ccncc3n(C)c2=O)c2ncccc12)C(=O)O]

In some embodiments, a method of treating a patient having an α4β7 integrin medicated disease or condition includes administering a solid form of a compound of Formula I. In some embodiments, a method of treating a patient having an α4β7 integrin medicated disease or condition includes administering a solid form of a compound of Formula III. In some embodiments, a method of treating a patient having an α4β7 integrin medicated disease or condition includes administering a solid form of a compound of Formula IV. In some embodiments, disclosed herein, the α4β7 integrin mediated disease or condition is an inflammatory disease. In some embodiments, the inflammatory disease or condition is a liver disease or a metabolic disease.

In some embodiments, a method of treating a patient having an inflammatory disease includes administering a solid form of a compound of Formula I.

In some embodiments, a method of treating a patient having an inflammatory disease includes administering a solid form of a compound of Formula III.

In some embodiments, a method of treating a patient having an inflammatory disease includes administering a solid form of a compound of Formula IV.

In some embodiments, a compound or composition disclosed herein is provided for the manufacture of a medicament for the treatment of a α4β7 integrin mediated disease or condition.

In some embodiments, the α4β7 integrin mediated disease or condition is an inflammatory disease. In some embodiments, the α4β7 integrin mediated disease or condition is a liver disease or a metabolic disease. In some embodiments, the α4β7 integrin mediated disease or condition is a rheumatology disease, such as rheumatoid arthritis (RA), systemic lupus erythematosus (SLE), lupus nephritis (LN), or osteoarthritis (OA). In some embodiments, the α4β7 integrin mediated disease or condition is Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, or Takayasu arteritis. In some embodiments, the α4β7 integrin mediated disease or condition is cutaneous lupus erythematosus (CLE) types: chronic CLE (including discoid), subacute CLE, acute CLE; seropositive or seronegative RA, juvenile idiopathic arthritis (JIA); primary osteoarthritis or secondary osteoarthritis, cervical and lumbar spinal osteoarthritis, hip osteoarthritis, knee osteoarthritis, or erosive osteoarthritis.

In some embodiments, the α4β7 integrin mediated disease or condition is a gastroenterology disease, such as ulcerative colitis (UC), or Crohn's disease (CD). In some embodiments, the α4β7 integrin mediated disease or condition is eosinophilic gastrointestinal disorders (EGIDs), such as eosinophilic esophagitis (EoE), eosinophilic gastroenteritis, eosinophilic colitis, or microscopic colitis. In some embodiments, α4β7 integrin mediated disease or condition is ulcerative proctitis, proctosignmoiditis, left-sided colitis, extensive colitis, pancolitis, ileocolitis, ileitis, gastroduodenal CD, jejunoileitis, or Crohn's (granulomatous) colitis.

In some embodiments, the α4β7 integrin mediated disease or condition is a pulmonology disease, such as idiopathic pulmonary fibrosis (IPF), or interstitial lung disease (ILD). In some embodiments, the α4β7 integrin mediated disease or condition is acute respiratory distress syndrome (ARDS), asthma; bronchiolitis obliterans, chronic obstructive pulmonary disease (COPD); Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis; bronchiectasis; primary ciliary dyskinesia; pneumonia pulmonary arterial hypertension (PAH); lymphangioleiomyomatosis; nonspecific interstitial pneumonia; cryptogenic organizing pneumonia; acute interstitial pneumonia; familial interstitial lung disease, or bleomycin induced pulmonary fibrosis.

In some embodiments, the α4β7 integrin mediated disease or condition is a hepatology disease. In some embodiments, the α4β7 integrin mediated disease or condition is metabolic dysfunction-associated steatohepatitis (MASH). In some embodiments, the GLP-1 mediated disease or condition is Primary sclerosing cholangitis (PSC), primary biliary cirrhosis (PBC), autoimmune hepatitis, alcoholic steatohepatitis (ASH), or alcoholic hepatitis. In some embodiments, the α4β7 integrin mediated or condition is chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), or Barrett's esophagus.

In some embodiments, the α4β7 integrin mediated disease or condition is a nephrology disease, such as diabetic kidney disease (DKD) (diabetic nephropathy). In some embodiments, the α4β7 integrin mediated disease or condition is chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 and type 2 diabetes mellitus, or hyperuricemia.

In some embodiments, the α4β7 integrin mediated disease or condition is a dermatology or allergic disease, such as, atopic dermatitis (AD). In some embodiments, the α4β7 integrin mediated disease or condition is atopic dermatitis, contact dermatitis, vitiligo, alopecia areata, acne, psoriasis, dermatomyositis, scleroderma, or morphea.

In some embodiments, the α4β7 integrin mediated disease or condition is an inflammatory disease or condition, such as adult-onset Still's disease, alcoholic hepatitis, alcoholic steatohepatitis, alcoholic liver disease, asthma, including allergen-induced asthma, bullous pemphigoid (BP) asthma, non-allergen induced asthma, allergies and allergic conditions such as allergic bronchopulmonary aspergillosis, allergic conjunctivitis, allergic encephalomyelitis, and allergic neuritis, food allergies, allograft rejection, alcoholic steatohepatitis (ASH), ANCA vasculitis, anti-glomerular basement membrane disease (Anti-GBM), antiphospholipid syndrome, aphthous stomatitis, appendicitis, arthritis, autoimmune diseases, atrophic thyroiditis, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, pernicious anemia (Addison's disease), and autoimmune thyroid disorders, autoinflammatory diseases, autosomal dominant polyscystic kidney disease (ADPKD), ankylosing spondylitis (AS), acute respiratory distress syndrome (ARDS), Bechet's disease or syndrome, bee sting-induced inflammation, Blau syndrome, bursitis, Barrett's esophagus, bleomycin induced pulmonary fibrosis, bronchiolitis obliterans, cardiac hypertrophy, gluten-sensitive enteropathy (Celiac disease), chemical irritant-induced inflammation, chorioretinitis, chronic atypical neutrophilic dermatosis with lipodystrophy and elevated temperature (CANDLE) syndrome, chronic obstructive pulmonary disease (COPD), chronic pancreatitis, chronic prostatitis, chronic recurrent multifocal osteomyelitis, cicatricial alopecia, colitis, complex regional pain syndrome, chronic intrahepatic or extrahepatic cholestatic disease, conjunctivitis, connective tissue disease, Connective tissue disease-associated interstitial lung disease (CTD-ILD), corneal ulcer, cryopyrin-associated periodic syndromes, cutaneous lupus erythematosus (CLE), cystic fibrosis, deficiency of the interleukin-1 receptor antagonist (DIRA), deficiency of IL36R antagonist (DITRA), dermatitis, diabetic kidney disease (DKD) (diabetic nephropathy), diverticulitis, discoid lupus erythematosus, drug induced delayed type cutaneous allergic reactions, encephalitis, esophagitis, eosinophilic gastrointestinal disorders (EGIDs), such as eosinophilic esophagitis (EoE), eosinophilic gastroenteritis, eosinophilic colitis, familial cold urticarial, familial Mediterranean fever, fistulizing Crohn's disease, giant cell arteritis, glomerulonephritis, gout, gouty arthritis, graft-versus-host disease (GVHD), granulomatous hepatitis, Guillain-Barre syndrome (GBS), Graves' disease, Hashimoto's thyroiditis, Henoch-Schanlein purpura, hidradenitis suppurativa (HS), hyaline membrane disease, hyperactive inflammatory response, hypereosinophilic syndrome (HES), hyperimmunoglobulinemia D with recurrent fever (HIDS), hypersensitivity pneumonitis (HP), immunoglobulin (IgA) nephropathies, IgG4-related disease, immune complex nephritis, immune thrombocytopenic purpura (ITP), inflammation, inflammation of the CNS, inflammatory bowel disease (IBD), inflammatory disease of the respiratory tract (upper or lower) such as inflammatory lung disease, bronchitis, sinusitis, inflammatory ischemic event such as stroke or cardiac arrest, inflammatory liver disease, inflammatory myopathy, inflammatory neuropathy, inflammatory pain, insect bite-induced inflammation, interstitial cystitis, iritis, irritant-induced inflammation, juvenile arthritis, juvenile rheumatoid arthritis, keratitis, kidney transplant rejection, kidney disease, kidney fibrosis, kidney insufficiency, leukocyte adhesion deficiency, Loeffler's syndrome, lupus, lupus nephritis (LN), liver fibrosis, liver steatosis, liver ischemia, lipid and lipoprotein disorders, mast cell activation syndrome, mastocytosis, meningitis, microscopic colitis, mixed connective tissue disease, morphea or morphea variants, Muckle-Wells syndrome (urticaria deafness amyloidosis), mucositis, myelitis, myocarditis, myositis, necrotizing enterocolitis, neonatal onset multisystem inflammatory disease (NOMID), nasal polyps, neovascular glaucoma, neuritis, non-alcoholic fatty liver disease (NAFLD), metabolic dysfunction-associated steatohepatitis (MASH), non-radiographic axial spondyloarthritis (nr-AxSpA), non-cystic fibrosis bronchiectasis (non-CFB), obstructive or chronic inflammatory disorders of the liver, ocular allergy, optic neuritis, organ transplant rejection, osteoarthritis (OA), otitis, pancreatitis, pancolitis, pelvic inflammatory disease, pemphigus vulgaris (PV), bullous pemphigoid (BP), pericarditis, periodontitis, PFAPA (periodic fever, aphthous stomatitis, pharyngitis, adenitis), plant irritant-induced inflammation, pneumocystis infection, pneumonia, pneumonitis, poison ivy/urushiol oil-induced inflammation, polyarteritis nodosa, polychondritis, polycystic kidney disease (PCKD), polymyalgia rheumatic, polymyositis, pouchitis, proctitis, proctosignmoiditis, psoriatic arthritis (PsA), pulmonary arterial hypertension (PAH), pulmonary fibrosis, pyogenic sterile arthritis, pruritus, reperfusion injury and transplant rejection, primary biliary cirrhosis (PBC), primary sclerosing cholangitis (PSC), Raynaud's syndrome, Reiter's disease, reactive arthritis, renal graft rejection, reperfusion injury, rheumatic carditis, rheumatic diseases, rheumatic fever, rheumatoid arthritis (RA), rhinitis, rhinitis psoriasis, sarcoidosis, Schnitzler syndrome, scleritis, sclerosis, such as systemic sclerosis (SSc), seborrhea, sepsis, septic shock, Sjogren's syndrome, inflammatory skin diseases or conditions, such as acne, alopecia areata, atopic dermatitis, rosacea, eczema, dermatitis, dermatitis endotoxemia, dermatomyositis, stasis dermatitis, Stevens-Johnson syndrome (SJS), skin irritation, skin rash, skin sensitization (contact dermatitis or allergic contact dermatitis), scleroderma, psoriasis, psoriasis vulgaris, psoriatic arthritis, spinal stenosis, spondyloarthropathies, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thyroditis, transplantation rejection, tubulointerstitial nephritis, tubular disfunction, Takayasu arteritis, toxic epidermal necrolysis, urticaria, uterine fibroids, uveitis, uveoretinitis, vasculitis, vasculitis (NHLBI), vitiligo, or Wegener's granulomatosis.

In some embodiments, the α4β7 integrin mediated disease or condition is a disease or condition, such as, acne, acid-induced lung injury, Addison's disease, adrenal hyperplasia, adrenocortical insufficiency, age-related macular degeneration, aging, alcoholic liver disease, Alzheimer's disease, angina pectoris, angiofibroma, anhidrotic ectodermal dysplasia, ascites, aspergillosis, atherosclerosis, atherosclerotic plaques, amyloidosis, amyotrophic lateral sclerosis (ALS), angioedema, acute myocardial infarction, antigen-antibody complex mediated diseases, alpha-1-antitrypsin deficiency, back pain, Bacillus anthracis infection, Bell's palsy, berylliosis, bone pain, burns, bullous pemphigoid, cancer, carpal tunnel syndrome, Castleman's disease, catabolic disorders, cataracts, cerebral aneurysm, complications of organ transplantation, corneal graft neovascularization, cryptococcosis, a non-malignant hyperproliferative disorder; a malignant hyperproliferative disorder, hepatocellular carcinoma; colon adenoma, polyposis, colon adenocarcinoma, breast cancer; pancreatic adenocarcinoma, chronic heart failure, chronic lung disease of prematurity, cardiometabolic syndrome, cardiovascular disease, cutaneous T cell lymphoma, diabetic macular edema, dyslipidemia, endometriosis, endotoxemia, eosinophilic GI disease (EGID), eosinophilic esophagitis (EoE), eosinophilic pneumonias, epicondylitis, epidermolysis bullosa, erythema multiforme, erythroblastopenia, familial amyloidotic polyneuropathy, fetal growth retardation, fibromyalgia, glaucoma, glioblastoma, glomerular disease, gut diseases, growth plate injuries, hair loss, herpes zoster and simplex, hypoplastic and other anemias, head injury, hepatitis A, B, C, D, and E, herpes; headache, hearing loss, heart disease, hemangioma, hemophilic joints, hereditary periodic fever syndrome, heritable disorders of connective tissue, Hodgkin's disease, Huntington's disease, hyperammonemia, hypercalcemia, hypercholesterolemia, hemolytic anemia, hepatitis, hip replacement, hypertropic bone formation, hypersensitivity pneumonia, hereditary fructose intolerance, hypertension, hyperuricemia, idiopathic demyelinating polyneuropathy, infectious diseases including viral diseases such as AIDS (HIV infection), ichthyosis, incontinentia pigmenti (IP, Bloch-Siemens syndrome), idiopathic thrombocytopenic purpura, infectious mononucleosis, ischemia/reperfusion, insulin resistance, joint replacement, kidney injury caused by parasitic infections, leptospirosis, lichen sclerosus (LS), lichen planus, Lambert-Eaton myasthenic syndrome, Lyme disease, liver failure, including acute liver failure, muscle wasting, muscular dystrophy, Marfan syndrome (MFS), meningioma, mesothelioma, multiple organ injury syndrome, myasthenia gravis (MG), myelodysplastic syndrome, metabolic syndrome, multiple sclerosis, nephrotic syndrome, neuropathological diseases, nuclear factor-kappa B essential modulator (NEMO) deficiency syndrome, obesity, Osler-Weber syndrome, osteogenesis imperfecta, osteonecrosis, osteoporosis, pachyonychia congenita, Paget's disease, Paget's disease of bone, Parkinson's disease, periodic fever, pertussis, primary pulmonary hypertension, pyoderma gangrenosum, pyogenic granuloma retrolental fibroplasias, peritoneal endometriosis, Prurigo nodularis, psychosocial stress diseases, pulmonary disease, pulmonary hypertension, respiratory distress syndrome, renal disease, retinal disease, retrolental fibroplasia, renal transplant rejection, renal protection against drugs inducing Fanconi's syndrome, respiratory tract illness caused by respiratory syncytial virus, rhinosinusitis; radiation induced fibrosis, sarcoidosis, severe pain, sleep apnea, scoliosis, sickle cell anemia, sports injuries, sprains and strains, sunburn, spinal cord injury, Sèzary syndrome, silica-induced disease (Silicosis), subarachnoid hemorrhage, tuberculosis, tumor necrosis factor (TNF) receptor associated periodic syndrome (TRAPS), thrombosis; traumatic brain injury, tissue transplant, complications from type 1 or type 2 diabetes, toxoplasmosis, thrombocytopenia, trachoma, vascular restenosis, ventilator induced lung injury, Whipple's disease, or 2,8-dihydroxyadenine nephropathy.

In some embodiments, the α4β7 integrin mediated disease or condition is a lipid and lipoprotein disorder; Type I Diabetes; Type II Diabetes; clinical complications of Type I and Type II Diabetes selected from the group consisting of diabetic nephropathy, diabetic neuropathy, diabetic retinopathy and other observed effects of clinically manifest long term Diabetes; Non-Alcoholic Fatty Liver Disease (NAFLD); Non-Alcoholic Steatohepatitis (NASH); metabolic dysfunction-associated steatohepatitis (MASH); obesity; a metabolic syndrome selected from the group consisting of combined conditions of dyslipidemia, diabetes and abnormally high body-mass index; acute myocardial infarction; acute stroke; or thrombosis which occurs as an endpoint of chronic obstructive atherosclerosis.

In some embodiments, the α4β7 integrin mediated condition is: a non-malignant hyperproliferative disorder; and a malignant hyperproliferative disorder selected from the group consisting of hepatocellular carcinoma, colon adenoma, and polyposis; colon adenocarcinoma; breast cancer; pancreas adenocarcinoma; Barrett's esophagus; or other forms of neoplastic diseases of the gastrointestinal tract and the liver.

In some embodiments, the α4β7 integrin mediated condition is metabolic dysfunction-associated steatohepatitis (MASH), primary sclerosing cholangitis (PSC), primary biliary cirrhosis (PBC), autoimmune hepatitis, alcoholic steatohepatitis (ASH), or alcoholic hepatitis.

In some embodiments, the α4β7 integrin mediated condition is Ulcerative colitis.

In some embodiments, the α4β7 integrin mediated condition is Crohn's disease.

Medicaments as referred to herein may be prepared by conventional processes, including the combination of a compound according to the present disclosure and a pharmaceutically acceptable carrier.

### Kits

Provided herein are also kits that include a compound or composition described herein and suitable packaging. In one embodiment, a kit further includes instructions for use. In one aspect, a kit includes a crystalline form of the disclosure, or composition including a crystalline form of the disclosure and a label and/or instructions for use of the compounds in the treatment of the indications, including the diseases or conditions, described herein.

Provided herein are also articles of manufacture that include a compound or composition described herein in a suitable container. The container may be a vial, jar, ampoule, preloaded syringe, and intravenous bag.

### Combination Therapy

In some embodiments, disclosed herein are oral dosage forms comprising a novel crystalline form of a compound of Formula I:

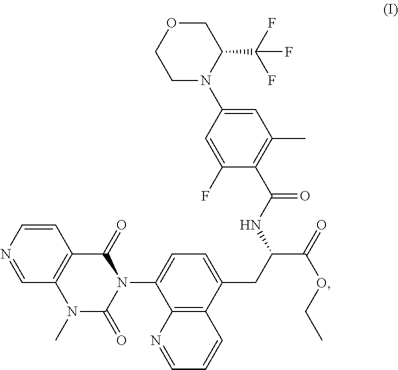

or a pharmaceutically acceptable salt thereof and at least one additional therapeutic agent.

In some embodiments, disclosed herein are oral dosage forms comprising a novel crystalline form of a compound of Formula III:

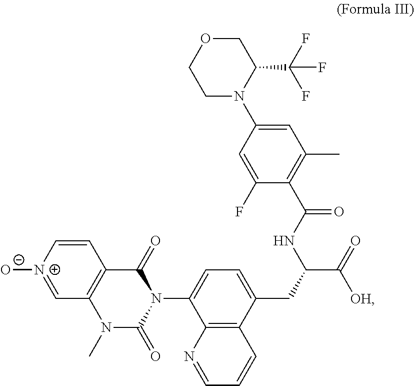

or a pharmaceutically acceptable salt thereof and at least one additional therapeutic agent.

In some embodiments, disclosed herein are oral dosage forms comprising a novel crystalline form of a compound of Formula IV:

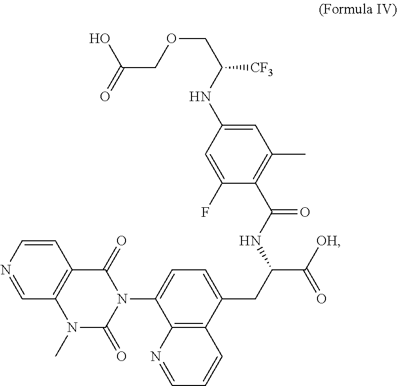 [SMILES: Cc1cc(N[C@H](COCC(=O)O)C(F)(F)F)cc(F)c1C(=O)N[C@@H](Cc1ccc(-n2c(=O)c3ccncc3n(C)c2=O)c2ncccc12)C(=O)O]

or a pharmaceutically acceptable salt thereof and at least one additional therapeutic agent.

In some embodiments, the oral dosage forms disclosed herein comprise novel crystal forms of Formula I, III, or IV or a pharmaceutically acceptable salt thereof and one, two, or three additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents. In some embodiments, a compound of Formula I is co-administered with one or more (e.g., one, two, three, or four) additional therapeutic agents. In some embodiments, the additional therapeutic agent includes an agent useful for modulating, treating, or preventing inflammation, such as a 1,3 beta glucanosyltransferase GAS2 modulator, 11-Beta hydroxysteroid dehydrogenase 1 inhibitor, 11-Beta hydroxysteroid dehydrogenase inhibitor, 12-Lipoxygenase inhibitor, 17 beta hydroxysteroid dehydrogenase 13 inhibitor, 2-Acylglycerol O-acyltransferase 2 inhibitor, 3 Ketoacyl CoA thiolase inhibitor, 40S ribosomal protein S4 stimulator, 5-HT 2a receptor agonist, 5-HT 2a receptor antagonist, 5-Lipoxygenase inhibitor, 6-Phosphofructokinase inhibitor, ACAT inhibitor, ACAT-2 inhibitor, Acetyl CoA carboxylase inhibitor, Acetyl CoA carboxylase-1 inhibitor, Acetyl CoA carboxylase-2 inhibitor, Acidic mammalian chitinase inhibitor, ACMSD gene inhibitor, Actin antagonist, Activin type-IIB receptor antagonist, Adenosine A1 receptor antagonist, Adenosine A2a receptor antagonist, Adenosine A3 receptor agonist, Adenosine A3 receptor antagonist, Adenosine kinase inhibitor, Adenylyl cyclase associated protein 1 inhibitor, Adiponectin receptor agonist, Adiponectin receptor modulator, Adiponectin receptor-1 agonist, Adiponectin receptor-2 agonist, Adiponutrin inhibitor, Adipose triglyceride lipase stimulator, Adrenergic receptor antagonist, Advanced glycosylation product receptor antagonist, AKT protein kinase stimulator, Albumin agonist, Albumin modulator, Aldosterone antagonist, Alkaline phosphatase modulator, Alpha 1 antitrypsin stimulator, Alpha 1 proteinase inhibitor, Alpha-amylase inhibitor, Alpha-glucosidase inhibitor, Alstrom syndrome protein 1 modulator, Aminopeptidase N modulator, Aminotransferase stimulator, AMP activated protein kinase alpha 2 stimulator, AMP activated protein kinase modulator, AMP activated protein kinase stimulator, Amylin receptor agonist, Amylin receptor antagonist, Amylin receptor modulator, Amyloid protein deposition inhibitor, Anaphase promoting complex subunit modulator, Androgen receptor agonist, Angiotensin converting enzyme 2 inhibitor, Angiotensin converting enzyme gene modulator, Angiotensin I receptor antagonist, Angiotensin II AT-1 receptor antagonist, Angiotensin II receptor antagonist, ANP agonist, AP1 transcription factor modulator, APOA1 gene stimulator, APOC3 gene inhibitor, APOE gene stimulator, Arginine deiminase stimulator, Arrestin domain containing protein 1 modulator, Aryl hydrocarbon receptor agonist, Asialoglycoprotein receptor 1 modulator, ATP citrate lyase inhibitor, Autophagy protein inhibitor, Autophagy protein modulator, Autophagy protein stimulator, Axin modulator, Bax protein stimulator, BC ketoacid dehydrogenase kinase inhibitor, Beta 2 adrenoceptor agonist, Beta 3 adrenoceptor agonist, Beta amyloid modulator, Beta secretase 2 inhibitor, Beta-catenin inhibitor, Bifunctional aminoacyl tRNA synthetase inhibitor, B-lymphocyte antigen CD19 modulator, B-lymphocyte stimulator ligand inhibitor, Bone morphogenetic protein-11 ligand, Bone morphogenetic protein-2 ligand inhibitor, Bone morphogenetic protein-4 ligand inhibitor, Bone morphogenetic protein-7 ligand, Bromodomain containing protein 2 inhibitor, Bromodomain containing protein 4 inhibitor, Cak tyrosine kinase receptor inhibitor, Calcineurin inhibitor, Calcitonin receptor agonist, Calcium channel inhibitor L-type, Calcium channel stimulator, Calpain inhibitor, Calpain-I inhibitor, Calpain-II inhibitor, Calpain-IX inhibitor, CAM kinase II inhibitor, Cannabinoid CB1 receptor antagonist, Cannabinoid CB1 receptor inverse agonist, Cannabinoid CB1 receptor modulator, Cannabinoid CB2 receptor modulator, Casein kinase II alpha inhibitor, Caspase inhibitor, Caveolin 1 inhibitor, CCAAT enhancer binding protein beta modulator, CCK receptor antagonist, CCL26 gene inhibitor, CCR2 chemokine antagonist, CCR3 chemokine modulator, CCR5 chemokine antagonist, CD122 modulator, CD158a antagonist, CD158b1 antagonist, CD158b2 antagonist, CD2 antagonist, CD3 antagonist, CD3 modulator, CD4 agonist, CD4 modulator, CD40 ligand inhibitor, CD40 ligand receptor antagonist, CD47 antagonist, CD80 antagonist, CDGSH iron sulfur domain protein modulator, Cell adhesion molecule inhibitor, Chitinase inhibitor, Chitotriosidase 1 inhibitor, Cholecystokinin CCK2 receptor antagonist, Chymase inhibitor, CIDEB gene inhibitor, Claudin 1 inhibitor, C-myc binding protein inhibitor, Collagen gene inhibitor, Collagen I antagonist, Collagen I modulator, Collagen III agonist, Collagen modulator, Complement C1q TNF-related protein modulator, Connective tissue growth factor ligand inhibitor, COT protein kinase inhibitor, CREB binding protein inhibitor, CRF-2 receptor antagonist, Cryptochrome modulator, CTGF gene inhibitor, CXCL10 gene inhibitor, CXCL11 gene inhibitor, CXCL9 gene inhibitor, CXCR1 chemokine antagonist, CXCR2 chemokine antagonist, CXCR3 chemokine antagonist, CXCR4 chemokine antagonist, CXCR7 chemokine agonist, Cyclin-dependent kinase-4 inhibitor, Cyclin-dependent kinase-6 inhibitor, Cyclooxygenase 2 inhibitor, Cyclooxygenase inhibitor, Cytochrome P450 2E1 inhibitor, Cytochrome P450 3A4 inhibitor, Cytochrome P450 7A1 inhibitor, Cytochrome P450 reductase inhibitor, Cytotoxic T-lymphocyte protein-4 stimulator, DGAT2 gene inhibitor, Diacylglycerol O acyltransferase 1 inhibitor, Diacylglycerol O acyltransferase 2 inhibitor, Dihydroceramide delta 4 desaturase inhibitor, Dihydroorotate dehydrogenase inhibitor, Dipeptidyl peptidase IV inhibitor, DNA cytosine-5 methyltransferase 1 inhibitor, DNA gyrase inhibitor, DNA methyltransferase inhibitor, Dopamine D2 receptor agonist, DYRK-1 alpha protein kinase inhibitor, E3 ubiquitin protein ligase COP1 modulator, E3 ubiquitin protein ligase ZNRF2 modulator, Ectonucleotide pyrophosphatase-PDE-2 inhibitor, EIF-2 alpha protein kinase stimulator, Endoglin inhibitor, Endothelial nitric oxide synthase stimulator, Endothelin ET-A receptor antagonist, Endothelin ET-B receptor antagonist, Eotaxin 2 ligand inhibitor, Eotaxin ligand inhibitor, Epha4 tyrosine kinase receptor stimulator, Ephb2 tyrosine kinase receptor inhibitor, Ephrin B2 ligand inhibitor, Epidermal growth factor receptor agonist, Epidermal growth factor receptor antagonist, Epidermal growth factor receptor modulator, Epoxide hydrolase inhibitor, Erbb2 tyrosine kinase receptor inhibitor, Erbb2 tyrosine kinase receptor modulator, Erythropoietin receptor agonist, Estradiol 17 beta dehydrogenase 1 inhibitor, Estrogen related receptor alpha modulator, Exendin 4 ligand, Extracellular signal related kinase modulator, Factor IIa antagonist, Farnesoid X receptor agonist, Farnesoid X receptor modulator, Fatty acid synthase inhibitor, FGF binding protein 3 stimulator, FGF receptor agonist, FGF-1 ligand, FGF1 receptor agonist, FGF19 gene stimulator, FGF-19 ligand, FGF21 gene modulator, FGF-21 ligand, FGF-21 ligand inhibitor, FGF-21 ligand modulator, FGF-3 ligand, FGFR1 oncogene modulator, FOXO1 transcription factor inhibitor, Free fatty acid receptor 1 agonist, Free fatty acid receptor 2 agonist, Free fatty acid receptor 3 agonist, Fructose 1,6 biphosphatase inhibitor, Fyn tyrosine kinase inhibitor, G protein coupled receptor 142 agonist, GABA A receptor antagonist, GABA A receptor modulator, GABA receptor agonist, GABA receptor modulator, Galanin receptor antagonist, Galectin-3 inhibitor, Gamma-secretase inhibitor, Gastric inhibitory peptide ligand, Gastric inhibitory polypeptide receptor agonist, Gastric inhibitory polypeptide receptor modulator, GCGR gene inhibitor, GDF-15 ligand, GDF-8 antagonist, GDNF family receptor alpha like agonist, GDNF receptor modulator, GHRH receptor agonist, Glucagon ligand, Glucagon receptor agonist, Glucagon receptor antagonist, Glucagon-like peptide 1 gene modulator, Glucagon-like peptide 1 receptor agonist, Glucagon-like peptide 1 receptor antagonist, Glucagon-like peptide 1 receptor modulator, Glucagon-like peptide 2 receptor agonist, Glucocorticoid receptor agonist, Glucocorticoid receptor antagonist, Glucokinase inhibitor, Glucokinase stimulator, Glucose 6-phosphate 1-dehydrogenase inhibitor, Glutamate decarboxylase modulator, Glutaminyl peptide cyclotransferase inhibitor, Glycogen synthase kinase-3 beta inhibitor, Glycosidase inhibitor, GP IIb IIIa antagonist, G-protein coupled bile acid receptor 1 agonist, G-protein coupled receptor 120 agonist, G-protein coupled receptor 84 antagonist, G-protein coupled receptor-119 agonist, G-protein coupled receptor-39 antagonist, Guanylate cyclase stimulator, Hedgehog protein inhibitor, Heparanase inhibitor, Hepatitis B structural protein inhibitor, Hepatitis C virus protein NS5A inhibitor, Hepatocyte growth factor receptor agonist, Hepatocyte growth factor receptor modulator, Hepatocyte nuclear factor-4 receptor modulator, Histone deacetylase inhibitor, Histone deacetylase-1 inhibitor, Histone deacetylase-2 inhibitor, Histone deacetylase-3 inhibitor, Histone lysine methyltransferase EHMT2 inhibitor, HIV gp41 protein modulator, HIV-1 gp120 protein inhibitor, HLA antigen inhibitor, HLA class I antigen A-2 alpha modulator, HLA class II antigen inhibitor, HLA class II antigen modulator, HMG CoA reductase inhibitor, Hormone sensitive lipase stimulator, HSD17B13 gene inhibitor, HSD17B13 gene modulator, Hydrolase inhibitor, IGF binding protein-3 inhibitor, I-kappa B kinase epsilon inhibitor, IL-1 receptor antagonist, IL-10 receptor agonist, IL-11 receptor antagonist, IL-12 receptor antagonist, IL-17 antagonist, IL-2 receptor agonist, IL-22 agonist, IL-8 receptor antagonist, Ileal sodium bile acid cotransporter inhibitor, Ileal sodium bile acid cotransporter modulator, Immunoglobulin G1 agonist, Immunoglobulin gamma Fc receptor agonist, Inducible nitric oxide synthase inhibitor, INHBE gene inhibitor, INS gene modulator, Insulin degrading enzyme inhibitor, Insulin induced gene protein stimulator, Insulin ligand, Insulin receptor agonist, Insulin receptor substrate-2 stimulator, Insulin sensitizer, Insulin-like growth factor 1 receptor agonist, Insulin-like growth factor 1 receptor antagonist, Insulin-like growth factor 1 receptor modulator, Integrin alpha-V/beta-1 antagonist, Integrin alpha-V/beta-3 antagonist, Integrin alpha-V/beta-3 modulator, Integrin alpha-V/beta-5 modulator, Integrin alpha-V/beta-6 antagonist, Integrin antagonist, Interferon alpha 2 ligand, Interferon alpha ligand, Interferon alpha ligand inhibitor, Interferon gamma ligand, Interleukin 11 ligand modulator, Interleukin 17 ligand inhibitor, Interleukin 18 ligand inhibitor, Interleukin-1 alpha ligand inhibitor, Interleukin-1 beta ligand inhibitor, Interleukin-2 ligand, IRE1 protein kinase inhibitor, Islet amyloid polypeptide inhibitor, Jaki tyrosine kinase inhibitor, Jak2 tyrosine kinase inhibitor, Jak3 tyrosine kinase inhibitor, Jun N terminal kinase inhibitor, Jun N terminal kinase-1 inhibitor, KCNA voltage-gated potassium channel-3 modulator, Kelch like ECH associated protein 1 inhibitor, Kelch like ECH associated protein 1 modulator, Ketohexokinase inhibitor, Killer cell Ig like receptor antagonist, Klotho beta stimulator, LanC like protein 2 modulator, Leptin receptor agonist, Leukocyte elastase inhibitor, Leukotriene A4 hydrolase inhibitor, Leukotriene CysLT1 receptor antagonist, Leukotriene D4 antagonist, Lipase inhibitor, Liver X receptor alpha inverse agonist, Liver X receptor antagonist, Liver X receptor beta inverse agonist, Liver X receptor modulator, LKB1 protein kinase stimulator, LOXL2 gene inhibitor, Lymphocyte function antigen-3 receptor modulator, Lyn tyrosine kinase stimulator, Lysophosphatidate-1 receptor antagonist, Lysyl oxidase homolog 2 inhibitor, Lysyl tRNA synthetase inhibitor, Macrophage mannose receptor 1 modulator, MAdCAM modulator, MAFA gene stimulator, MALT protein 1 inhibitor, MAP kinase kinase 4 inhibitor, MARC1 gene inhibitor, Mas-related G-protein receptor D agonist, Matrix metalloprotease inhibitor, MEKK-5 protein kinase inhibitor, Membrane copper amine oxidase inhibitor, Menin inhibitor, Metalloprotease-9 stimulator, Methionine aminopeptidase-2 inhibitor, Mitochondrial carrier family modulator, Mitochondrial pyruvate carrier 2 inhibitor, Mitochondrial pyruvate carrier inhibitor, Mono ADP ribosyltransferase sirtuin-6 inhibitor, Mono ADP ribosyltransferase sirtuin-6 modulator, Motile sperm domain protein 2 inhibitor, MST-1 protein kinase inhibitor, mTOR complex 1 inhibitor, mTOR inhibitor, Myeloid lymphoid leukemia protein inhibitor, Myeloperoxidase inhibitor, NACHT LRR PYD domain protein 3 inhibitor, NAD-dependent deacetylase sirtuin stimulator, NAD-dependent deacetylase sirtuin-1 inhibitor, NAD-dependent deacetylase sirtuin-1 stimulator, NADH quinone oxidoreductase stimulator, NADPH oxidase 1 inhibitor, NADPH oxidase 4 inhibitor, NADPH oxidase inhibitor, Natriuretic peptide receptor B agonist, Nek-7 protein kinase inhibitor, Neuropeptide Y2 receptor agonist, NFE2L2 gene inhibitor, Nicotinamide N methyltransferase inhibitor, Nicotinamide phosphoribosyltransferase inhibitor, NK1 receptor antagonist, NLRP3 gene inhibitor, NMDA receptor antagonist, Notch antagonist, NOTCH1 gene inhibitor, NR1H3 gene inhibitor, Nuclear erythroid 2-related factor 2 stimulator, Nuclear factor kappa B modulator, Nuclear receptor 5A2 agonist, Nuclear receptor 5A2 modulator, Nucleoside reverse transcriptase inhibitor, Opioid receptor kappa agonist, Opioid receptor mu antagonist, Ornithine decarboxylase inhibitor, Orphan nuclear hormone receptor NR4A1 antagonist, Orphan nuclear receptor NURR1 agonist, OX-40 receptor antagonist, Oxyntomodulin ligand, P2X7 purinoceptor modulator, P2Y12 purinoceptor antagonist, P2Y13 purinoceptor stimulator, p38 MAP kinase inhibitor, PACAP ligand, PACAP type I receptor agonist, Palmitoyl protein thioesterase 1 inhibitor, Pancreatic beta cell growth factor modulator, Patatin like phospholipase protein modulator, PAX4 gene stimulator, PDE 3 inhibitor, PDE 4 inhibitor, PDGF receptor beta modulator, PDX1 gene stimulator, Peptide YY ligand, Peptide YY ligand modulator, Peptidyl-prolyl cis-trans isomerase A inhibitor, Peptidyl-prolyl cis-trans isomerase B inhibitor, Peptidyl-prolyl cis-trans isomerase D inhibitor, Phenylalanine hydroxylase stimulator, Phosphatase stimulator, Phosphatidylinositol 3 kinase subunit 3 inhibitor, Phospholipase C inhibitor, Phosphoric diester hydrolase inhibitor, Picolinate carboxylase inhibitor, Plasma retinol binding protein inhibitor, PNPLA3 gene inhibitor, PNPLA3 gene modulator, Potassium channel inhibitor, PPAR agonist, PPAR alpha agonist, PPAR delta agonist, PPAR delta antagonist, PPAR gamma agonist, PPAR gamma coactivator 1-alpha stimulator, PPAR gamma modulator, PPAR gamma partial agonist, PPAR modulator, Pregnane X receptor agonist, Programmed cell death ligand 1, Programmed cell death ligand 1 modulator, Programmed cell death protein 1 inhibitor, Proprotein convertase PC9 inhibitor, Proprotein convertase PC9 modulator, Protease-activated receptor-2 antagonist, Proteasome beta-8 subunit modulator, Proteasome inhibitor, Protein cereblon modulator, Protein disulfide isomerase A4 inhibitor, Protein kinase C alpha modulator, Protein NOV homolog modulator, Protein tyrosine phosphatase-1B inhibitor, Protein tyrosine phosphatase-2C inhibitor, PTGS2 gene inhibitor, PTPN1 gene inhibitor, Pyruvate dehydrogenase kinase inhibitor, Regenerating islet-derived protein 3a stimulator, Relaxin agonist, Relaxin receptor 1 agonist, Remodeling and spacing factor 1 inhibitor, Renin inhibitor, Resistin ligand inhibitor, Retinoic acid receptor beta agonist, Retinoic acid receptor gamma antagonist, Retinoid X receptor alpha agonist, Retinoid X receptor modulator, Rev protein modulator, Rho associated protein kinase 2 inhibitor, Ribosomal protein S6 kinase-1 inhibitor, RNA polymerase inhibitor, Secretin antagonist, Seprase inhibitor, Seprase modulator, Serine protease inhibitor, Serine threonine protein kinase 25 inhibitor, Serine threonine protein kinase TBK1 inhibitor, Serpin B13 inhibitor, SERPINA1 gene inhibitor, Serum protein modulator, Serum response factor inhibitor, SIRT6 gene stimulator, SMAD inhibitor, SOD2 gene stimulator, Sodium bile acid cotransporter inhibitor, Sodium glucose transporter-1 inhibitor, Sodium glucose transporter-2 inhibitor, Solute carrier family inhibitor, Somatostatin 2 receptor antagonist, Somatostatin 5 receptor antagonist, Sphingolipid delta 4 desaturase DES1 inhibitor, Sphingosine kinase 1 inhibitor, Sphingosine-1-phosphate receptor-1 antagonist, Sphingosine-1-phosphate receptor-4 antagonist, SREBP cleavage activating protein inhibitor, SREBP transcription factor 1 inhibitor, SREBP transcription factor 2 inhibitor, SREBP1C gene inhibitor, STAT-1 modulator, STAT-3 inhibitor, Stearoyl CoA desaturase-1 inhibitor, Stellate protein kinase inhibitor, Subtilisin inhibitor, Sucrose alpha-glucosidase inhibitor, Synaptic vesicular amine transporter inhibitor, Syncytin 1 inhibitor, T cell receptor agonist, T cell receptor modulator, T cell surface glycoprotein CD28 inhibitor, TACE inhibitor, Tafazzin stimulator, Taste receptor type 2 agonist, Tau protein inhibitor, T-cell surface glycoprotein CD1d modulator, T-cell surface glycoprotein CD8 modulator, T-cell transcription factor NFAT modulator, Tec tyrosine kinase inhibitor, Telomerase stimulator, TERT gene modulator, TGF beta 1 ligand inhibitor, TGF beta 1 ligand modulator, TGF beta 3 ligand inhibitor, TGF beta ligand inhibitor, TGF beta ligand modulator, TGF beta receptor antagonist, TGFB1 gene inhibitor, Thioredoxin interacting protein inhibitor, Thioredoxin reductase inhibitor, Thrombospondin-1 ligand inhibitor, Thromboxane A2 antagonist, Thyroid hormone receptor agonist, Thyroid hormone receptor beta agonist, Tissue transglutaminase inhibitor, TLR antagonist, TLR modulator, TLR-2 antagonist, TLR-4 agonist, TLR-4 antagonist, TLR-9 antagonist, TNF alpha induced protein 3 stimulator, TNF alpha ligand inhibitor, TNF antagonist, TNF binding agent, TNF related apoptosis inducing ligand, Topoisomerase IV inhibitor, TRAIL receptor agonist, TRAIL-1 receptor modulator, TRAIL-2 receptor antagonist, Transcriptional regulator protein BACH1 modulator, TRIM33 gene modulator, Tripartite motif containing protein 72 inhibitor, TRP cation channel V1 antagonist, Trypsin inhibitor, Tumor necrosis factor 13C receptor antagonist, Tumor necrosis factor 14 ligand inhibitor, Type I TNF receptor antagonist, Tyrosine kinase receptor modulator, Tyrosine phosphatase substrate 1 inhibitor, Ubiquitin ligase modulator, Uncoupling protein modulator, Urate anion exchanger 1 inhibitor, Urocortin II ligand, Vasopressin V1 receptor agonist, Vasopressin V1A partial agonist, Vasopressin V1a receptor agonist, Vasopressin V1a receptor antagonist, Vasopressin V2 receptor antagonist, VEGF receptor modulator, VEGF-A ligand inhibitor, VEGF-B ligand inhibitor, Vimentin inhibitor, Vitamin D3 receptor agonist, Wnt ligand, Wnt ligand modulator, Wnt-1 induced signal pathway protein 1 inhibitor, Xanthine oxidase inhibitor, ZSCAN4 gene stimulator, or 1 Aminocyclopropane carboxyl synthase inhibitor.

In some embodiments, disclosed herein comprise novel crystal forms of Formula I or a pharmaceutically acceptable salt thereof, is co-administered with one or more agents useful for the treatment and/or prophylaxis of a heptatologic condition, such as NAFLD, or MASH. Non-limiting examples of such agents include glucagon-like peptide-1 receptor agonists (GLP-1 RA, e.g., liraglutide, semaglutide, tirzepatide), sodium-glucose cotransporter-2 inhibitor (SGLT2i), thiazolidinediones (pioglitazone) and vitamin E.

In some embodiments, disclosed herein comprise novel crystal forms of Formula I or a pharmaceutically acceptable salt thereof, is co-administered with one or more agents useful for the treatment and/or prophylaxis of a heptatologic condition, such as PBC or PSC. Non-limiting examples of such agents include ursodeoxycholic acid or obeticholic acid.

Is some embodiments, the additional therapeutic agent includes an agent useful for modulating, treating, or preventing inflammation, such as a [18F]-TZ-Z-09591, [68Ga]Ga-DO3A-S01-GCG, 1,5-bisphenylpyrazole derivative, 11betaHSD1 inhibitors, 4P Exenatide, 5-aminolevulinic acid hydrochloride+sodium ferrous citrate, 68Ga-BOT1712, A-2906, A-4368, AAA-10, AAV8-FGF19 variant M70, AAV-Ex-4, AB-004, AB-03, AB-04, ABA-201, abatacept, ABP2111-Na, ABP-6016, AC-261066, acarbose, acarbose+metformin, ACE-1332, acetylcysteine, ACQT-1001, ACQT-1127, ACT-777991, AD-214, AD-219, AD-222, AD-301, adalimumab, ADC-001, ADD-16, ADi-100, adiponectin agonists, adipose-derived stem cell therapy, ADO-09, AdoShell Islets, ADPO-002, ADPT-02, ADR-001, AER-501, AER-601, AG-019, AGEX-BAT1, AGN-242266, AGS-500, AGTX-2003, AGTX-2004, AKS-107, AKS-440, aldafermin, aldesleukin, ALD-R491, ALE-C04, ALE-F02, ALF-301, ALF-5755, ALG-055009, allogeneic blood-derived CD34-positive adult pluripotent stem cells, allogeneic human islets of Langerhans, allogeneic human pancreatic islets of Langerhans, allogeneic human umbilical cord mesenchymal stem cells, allogeneic mesenchymal stem cell therapy, allogeneic UC-MSC therapy, allogeneic umbilical cord mesenchymal stem cells, allogenic umbilical cord-derived mesenchymal stem cell therapy, ALN-KHK, ALN-PNP, alogliptin, alogliptin benzoate+metformin hydrochloride, alogliptin benzoate+metformin hydrochloride XR, alogliptin benzoate+pioglitazone hydrochloride, alpha glucosidase inhibitors, alpha-1 antitrypsin, alpha-v/beta-6 integrin inhibitors, ALS-L1023, ALT-100, ALTSuLIN, ALY688-SR, AM-510, AMA-002, AMP-activated protein kinase inhibitors, AMPK activators, AMS-III-1086, AMTX-100, AMTX-100, amyloid protein deposition inhibitor, anagliptin, anagliptin+metformin, AND-9, ANG-4201, antagomiR-144, anti-BMP-2/4 monoclonal antibodies, anti-CXCR3 mAb, anti-F1 gene therapy, anti-fibrotic macrophage cell therapy, anti-TAGE monoclonal antibody, anti-VEGF-B antibody, Antral, AP-025, AP-026, apabetalone, APB-R3, APH-012, aprocitentan, APX-311, AR-882, ARO-HSD, ARO-INHBE, ARO-PNPLA3, AS-100283, AS-1501, ASC-41, ASC-47, aspirin trelamine hydrochloride, AT-247, AT-278, AT-299, ATB-1013, Atenas, ATGC-300A, atorvastatin+metformin, ATR-127, ATR-258, Atrosimab, ATX-304, autologous bone marrow mononuclear cells, autologous leukocyte cell therapy, autologous tolerogenic dendritic cell therapy, autologous tolerogenic dendritic cell vaccine, autologous T-reg cell therapy, AUX-101, AVD-1001, avenciguat, AVM-0703, AVO-101, AVO-1681, AVT-001, AX-0601, AX-0810, AX-2911, AXA-1125, AZD-1656, AZD-1705, AZD-2389, AZD-2693, AZD-9550, azemiglitazone potassium, B-1344, B-1654, baricitinib, basal insulin, BBT-877, BC-0306, BCKDK inhibitor, BEBT-503, BEBT-508, beinaglutide, belapectin, belimumab, bempedoic acid, berberine chloride, berberine ursodeoxycholate, bermekimab, bersacapavir, bersiporocin dihydrochloride, bertilimumab, bexagliflozin, bexotegrast, Bezafibrate, bezafibrate+obeticholic acid, BF-114, BG-148, BGM-0504, BGT-002, BHD-1028, BI-3006337, BI-3231, BI-765423, BIIB-110, BioChaperone AsPram, BioChaperone Combo, BL-001, BLD-2660, BLD-3051, BLR-200, BMN-255, BMS-687681, BMS-820132, BNC-1602, BOS-580, BOT-1712-IFNg-SD, BP-103, BR-1019, BR-3003, BR-3005, BRL-186616, BRL-191118, BRL-201009, bromocriptine, BRS-201, BS-1801, BTI-320, budesonide, BX-002, BX-003, BXT-102, BX-U004, C-455, cadisegliatin, cagrilintide, cagrilintide+semaglutide, canagliflozin, canagliflozin+metformin IR, canagliflozin+metformin XR, canakinumab, cannabidiol, cannabinoid 1 receptor antagonists, CAR Treg-cell therapy, CB-0406, CB-4211, CB5138-3, Cbi-Ex4, CBL-514, CBW-511, CC-90001, CCX-872, CD4 antigen modulator/CD8 antigen modulator/T cell receptor modulator, celastrol, cell therapy, Cellgram-LC, CELZ-001, CELZ-100, CELZ-101, CELZ-201, cenicriviroc, CGT-8012, chiglitazar, CHIN-117, CHM-273S, cibinetide, CIDEB siRNA therapy, cilofexor+firsocostat, cilofexor tromethamine, citric acid monohydrate, CJC-1134-PC, CKD-371, CKD-378, CKD-379, CKD-383, CKD-386, CKD-389, CKD-393, CKD-398, class I HDAC inhibitors, CLBS-03, clesacostat+ervogastat, CM-101, CM-272, CNP-103, CNP-104, cofrogliptin, colesevelam, collagen I translation inhibitors (liver fibrosis), Anima Biotech, corticotropin releasing factor receptor-2 inhibitors, CPL-207-280, CRB-486, CS-0159, CS-17919, CS-27109, CT-02, CT-388, CT-868, CT-996, CTX-211, CUD-005, cudetaxestat sodium, CUE-301, CVB1 vaccine, CVI-301, CXCL9/CXCL10/CXCL11 gene therapy, CYRS-101, CYRS-MD02, CYTX-100, D4-201-01, D-745, D-759, DA-1241, DA-1726, DA-302168S, DA-5221-T, dalpiciclib, danuglipron, dapagliflozin+glimepiride+metformin hydrochloride, dapagliflozin+linagliptin, dapagliflozin+metformin, dapagliflozin+sitagliptin, dapagliflozin+sitagliptin+metformin hydrochloride, dapagliflozin+teneligliptin, dapagliflozin+vildagliptin, dapagliflozin citrate, dapagliflozin ER, dapagliflozin propanediol, dapagliflozin propanediol+sitagliptin phosphate, dapansutrile, DBI-500, DC-291407, DCR-CLD, DCR-LIV2, DD-01, DD-02, DD-03, DD-15, denatonium acetate monohydrate, dendritic cell-based microsphere vaccine, denifanstat, Des-1 inhibitor, DFV-890, DiabeCell, Diamyd, DLA bispecific antibody program, DLBS-3233, DM-1050, D-methyldopa, DNX-314, dorzagliatin, DR-10624, DT-109, dual agonist combination 1 therapy, dual agonist combination 3, dual GLP1/glucagon receptor agonists, dual PPAR-alpha/gamma agonists, dual PPAR-gamma/delta modulators, Duglow, dulaglutide, DWC-202001, DWC-202002, DWC-202213, DWJ-1525, DWJ-1563, DWP-457, DWRX-5003, EA-3571, EBX-102, ECC-0509, ECC-4703, ECC-5004, ecnoglutide, edaravone, efinopegdutide, eflornithine, efpeglenatide, efruxifermin, elafibranor, elebsiran, elobixibat, EM-101, Enterome, EMI-137, empagliflozin, empagliflozin+linagliptin, empagliflozin+linagliptin+metformin XR, empagliflozin+metformin, empagliflozin+metformin extended-release, enavogliflozin, ENB-106, ENB-110e, ENC-201, encapsulated Melligen cell therapy, ENERGI-F702, engineered regulatory T cell therapy, ENN-0403, ENT-001, epeleuton, EPGN-696, Epi-13, ERp44-adiponectin interactions modulators, ertugliflozin, ertugliflozin+metformin, ertugliflozin+sitagliptin, ervogastat, ETX-291, ETX-312, evexomostat, evogliptin, evogliptin+dalpagliflozin+metformin, evogliptin+metformin XR, Exd-391209, exenatide, exendin-4-Fc fusion protein, exendin-4-human serum albumin fusion protein, EXG-34217, exosome based therapeutic, extended release glipizide, EYP-002, ezurpimtrostat, F-573, farnesoid X receptor agonists, fazirsiran sodium, FB-102, FB-1603, FB-1807, FBPase inhibitors, fenofibrate+atorvastatin, fezagepras, FFAR4 agonists, FGF21 gene therapy, FIA-586, FIB-918, FIB-992, firsocostat, fluasterone, fluorofenidone, FM-101, foralumab, foscenvivint, fotagliptin benzoate, FOXO1 inhibitor program, FP10.47, fresolimumab, frexalimab, FRTX-02, FSI-965, FT-4101, Fuzheng Huayu capsule, FXR agonists, FXR-314, G3PP activator program, G-49, GABA+antigen based therapy, GAD-65, Gal-300, galectin-3 inhibitors, gallium (68Ga) rofapitide tetraxetan, GalNAc-conjugated siRNA therapeutic, GARV-AAV2-A20, GB-7001, G2GBio, GDD-3898, GDNF, gemigliptin+dapagliflozin, gemigliptin+metformin HCl, gemigliptin+rosuvastatin, gemigliptin tartaric acid, GEN-1503PR, GF-1002, GH-509, GI-210, GKAC, GL-0034, glargine biosimilar, glibenclamide, gliclazide, glimepiride, glimepiride+extended-release metformin hydrochloride, glimepiride+metformin, glimepiride+vildagliptin+metformin, GLP-1 analog-COL3A1 fusion protein, GLP-1 analogs, GLP-1 receptor agonist, GLP-1/exendin-4-Fc fusion proteins, GLP-1/GIP receptor agonist, GLP-1-Fc-PYY, glucagon-like peptide-1 analog, glucose responsive smart basal insulin, glutazumab, GLY-200, GLY-POL, GM-60106, GM-90194, GMA-106, GMA-107, GMA-10X, GMA-10Y, GNF-2133, GNS-3595, golexanolone, golimumab, gosogliptin, GPR39 antagonists, GPX-002, GPX-003, GR-012, GR-018, GR-019, GRI-0124, GS-300, GS-834356, GSBR-1290, GST-HG-151, GTX-011, GV-101, GX-G6, GXHPC-1, GXIPC-1, GZR-101, GZR-18, GZR-4, HB-1085, HB-601, HCL-001, HD-1916, HD-7671, HDM-1002, HDNO-1605, HEC-192334, HEC-44616, HEC-88473, HEC-96719, HepaStem, heterologous liver cell therapy, HGR-4113, HJ-178, HJC-0416, HK-1, HK-660S, HL-012MA, HL-08, HLA-DQ2/DQ8 inhibitors, HM-12460A, HM-15136+efpeglenatide, HM-15211, HM-15275, HNF4A mRNA therapeutics, HP-515, HPD-001, HPG-1860, HPG-5119, HPG-7233, HPN-01, HPN-F01, HPP-3033, HR-17031, HR-19042, HR-20033, HRS-7535, HRS-9531, HRX-0215, HRX-0701, HS-10356, HS-10501, HS-20004, HS-20094, HSD17B13 inhibitors, HSD17b13i, HSK-31679, HSK-34890, HT-201, HT-202, HTL-30023, HTPEP-002, HU-6, human albumin, human amniotic exosomes, human FGF-1, human insulin biosimilar, human plasma gelsolin, human pluripotent embryonic stem cell-derived pancreatic islet cells, human pluripotent stem cells derived allogenic hepatocytes, humanin analogs, HX-100101-1, HX-1171, HY-209, HYBR-011, hydrogel-exenatide, hydronidone, hydroxyl dendrimer-alendronate conjugate, hymecromone, hypoglycemic agents, HZ-010, HZ-012, i2o-107, i2o-110, i2o-120, ianalumab, IAPP inhibitor, ibrigampar, Ibutamoren, icomidocholic acid, icosabutate, icovamenib, ID-110521156, ID-11903, ID-119050134, IDG-16177, IDL-2965, IFNalpha kinoide, h-key/MHC class II epitope hybrid peptide immunomodulator peptide vaccines, IL-1512, IL-2 fusion protein, ILV-001, IM-102, IMC-S118AI, IMCY-0098, imeglimin, IMG-1, IMM-124-E, IMM-H014, ImmTOR+PDC-E2, IMT-001, IN-A004, IN-A010, INI-822, INS-068, insluin lispro, insparin, insulin, insulin aspart, insulin degludec, insulin degludec+insulin aspart, insulin degludec+liraglutide, insulin degrading enzyme inhibitors, insulin efsitora alfa, insulin glargine, insulin glargine+lixisenatide, insulin icodec, insulin lispro, insulin lispro+exenatide, insulin lispro+pramlintide, insulin mouth rinse, insulin tregopil, insulin-producing stem-islet beta cell hybrids, Insuman Implantable, INT-787, interferon alfa-2a follow-on biologic, interferon alfa-n1, interferon gamma follow-on biologic, INV-002, INVA-8001, invopressin, IOA-289, ION-224, ION-769357, IONIS-GCGRRx, IOT-022, IPG-008, IPN-60250, ipragliflozin, ipragliflozin+sitagliptin, iPSC cell therapy, iPSC-derived pancreatic islet cell therapy, IRE1alpha inhibitors, iscalimab, ISM-001, ITCA-1601, iTOL-101, iTOL-102, ITV-1, IVB-001, ivonescimab, iXB-401, J2H-1702, JAG-301, JMT-202, JP-2266, JT-003, JTT-251, JTT-662, JW-0201, K-757, K-833, kamuvudines, KBL-982, KBLP-004, KBP-336, KD-4002, KD-4004, KGYY-15, KH-629, KH-805, KH-806, KN-056, KQ-791, KSHB-005, KT-A112, KT-A522, KT-A832, Kv1.3 ion channel mAbs, KY-41111, KYLO-0603, L-47, LABP-111, LABthera-006, ladarixin, LAE-103, LAE-104, LAE-105, LAE-106, laflunimus, lanifibranor, lapretolimod, larsucosterol, LB-P7, LB-P8, LBS-009, LC-542019, LEM-S402, leronlimab, leucine+metformin+sildenafil, LG-203003, L-glutamine, LH-1801, licogliflozin bis(prolinate), linagliptin, linagliptin+dapagliflozin+metformin hydrochloride, linagliptin+metformin hydrochloride, linagliptin+metformin XR, linerixibat, liraglutide, liver disease/non-alcoholic steatohepatitis therapy, lixisenatide, LM-011, L-methyldopa, LN-3118, LNP-CDP mRNA therapy, lobeglitazone+metformin, lobeglitazone+sitagliptin, LP-342, LPCN-1148, LPS-001, LPXT-007, LR-1, LR-19018, LR-19131, LR-20022, LRH-1 modulators, LUNAR-TKD, luseogliflozin hydrate, LXR inverse agonists, LY-3493269, LY-3522348, LY-3532226, LY-3537021, LY-3549492, LY-3849891, LY-3885125, LY-3938577, LY900027, LYS-006, M-008, M-43, maralixibat chloride, maraviroc, masoprocol, mazdutide, MB-204, MB-N-008, MB-X01Y03, mCLC-846, mecasermin, memantine derivatives, MET-409, metformin, metformin+glibenclamide, metformin glycinate, metformin hydrochloride, metformin XR+valsartan+atorvastatin, MFC-0101, MFC-0102, MHS-552, mibavademab, microbiome-based live bacterial probiotic, microparticle vaccine, miglitol, miR-132 antisense oligonucleotide inhibitor, miricorilant, MI-S4, mitiglinide, mitiglinide+metformin, mitiglinide+voglibose, mitiperstat, mitochondrial uncouplers, MK-1092, MK-3655, MK-6204, MK-7480, MKP-10241, MLX-0800, MLX-5000, MLX-7000, MNO-863, monlunabant, MORF-627, mosedipimod, MPC-300-IV, MRG-229, mRNA-6981, mRNA-LNPs expressing EGF and HGF, MT-1002, MT-2002, MT-2004, MTX-101, MTX-463, MTX-474, MWN-101, MWN-102, myonectin, NA-941, naltrexone, namodenoson, nanofitins, nateglinide, nateglinide+metformin hydrochloride, NC-101, NDC-0009, necroptosis inhibitors, nerigliatin, netakimab, netazepide, NeuLiv, neuropeptide Y2-receptor agonists, NEXI-005, next generation peptidomimetic and tissue transglutaminase inhibitors, next generation smart glucose-responsive insulin, NGM-395, NI-203, nibrozetone, nicotinamide N-methyltransferase inhibitor, nimotuzumab, ninerafaxstat trihydrochloride monohydrate, NIPEP-CARE, nisotirostide, nitazoxanide, NJA-005, NKTT-320, NLRP3 gene therapy, NMX-2, NN-1471, NN-1535, NN-1845, NN-6177, NN-6561, NN-6581, NN-6582, NN-9041, NN-9541, NNC-0113-6856, NNC0113-6860, NNC0113-6861, NNC0113-6891, NNC-0194-0499, NNC-0472-0147, NNC0480-0389, NNC-0519-0130, NNC-0650-0013, NNC6022-0001, NNC-965, non-alcoholic fatty liver disease agent, norucholic acid, NovDB2, NovFS, NOX inhibitors, NOX4 inhibitors, NP-011, NTCP inhibitor, nucleoside-modified TERT mRNA, NV-422, NV-556, NX-9001, 0-01, OATD-01, obefazimod, obeticholic acid, obeticholic acid magnesium, OBM-P01, odevixibat, OGB-21501, OGB-21502, olmesartan+dapagliflozin, oltipraz, OLX-701, OLX-702A, omarigliptin, OPC-163493, OPT-101, ORBCEL-C, orforglipron, ORMD-0801, ORMD-0801+ORMD-0901, ORMD-0901, OsrHSA, otelixizumab, Oxy-210, P-11, pancreatic beta cell modulators, PanINSULA, PAR2 inhibitor pepducins, PB-718, PBI-4547, PEC-Direct, PEC-EnCap, pegapamodutide, pegargiminase, pegbelfermin, PEG-exenatide, peginterferon alfa-2a, peginterferon alfa-2b, PEG-loxenatide, pegozafermin, PEGylated exenatide, pemafibrate, pemafibrate+tofogliflozin, pemvidutide, Peptide, peptide-loaded nanoparticles, petrelintide, PF-06835919, PF-06954522, PF-07853578, PGC-fatylated terlipressin, PGN-0B2, PHIN-214, PHP-303, piclidenoson, pioglitazone, pioglitazone+extended-release metformin, pioglitazone+glimepiride, pioglitazone+metformin, pioglitazone+teneligliptin, PKX-001, PLN-1474, PNPLA3 inhibitor, PPAR-gamma agonists, pramlintide+exenatide, pramlintide+human insulin, preimplantation factor, PRIM-DJ2727, PRL-002, PRO-20, ProAgio, proglumide, Prolastin, proline henggliflozin+retagliptin phosphate+metformin hydrochloride, ProTrans, prusogliptin, PRV-101, PS-1, psilocybin, PSTvl, PT-001, PT-002, PTG-007, PTG-020, PTPN1 siRNA therapy, PTUPB, PVT-101, PXL-065, PXL-770, PXS-5382, pyrazole carboxamide analogs, PZH-2109, Qaialdo, QBT-002, QLR-12018, QPCT inhibitor, QRB-001, quisovalimab, R-0737072, R-2487, radiolabelled FAPI-46, rapirosiran, RAY-001, RAY-002, RAY-1225, RBP4 antagonist, RCYM-001, rE-4, recombinant angiotensin converting enzyme 2 (ACE-2) gene therapy/AAV vector, recombinant CTRP12 protein, remogliflozin etabonate, remogliflozin etabonate+teneligliptin, remogliflozin etabonate+vildagliptin, remogliflozin etabonate+vildagliptin+metformin, rencofilstat, REP-2139, repaglinide, repaglinide+metformin, repaglinide+metformin hydrochloride, RES-010, ReS39-I program, resmetirom, retagliptin, retatrutide, RG-125, rGDF11, RGT-028, RGT-075, RhuDex, rifaquizinone, rifaximin, rilparencel, ritlecitinib, RJ-4287, RJVA-001, RMD-1201, rosiglitazone, rosiglitazone+glimepiride, rosiglitazone maleate+metformin hydrochloride, rosiglitazone sodium, rosuvastatin+telmisartan+amlodipine, RP-005, RSVI-301, RT-200, RTX-001, RTX-T1D, Ryzodeg, RZ-629, SAB-142, SAL-0112, SAL-067, SAMiRNA program, SAMiRNA-AREG, santamarin derivatives, saRNA therapeutics, saroglitazar magnesium, saxagliptin, saxagliptin+dapagliflozin, saxagliptin+dapagliflozin+metformin hydrochloride, saxagliptin+extended-release metformin, saxagliptin+metformin, SBI-102, SBP-302, SBT-11-5301, SC-451, SCO-094, SCO-116, SCO-267, SCT-5-27, second generation chitotriosidase 1 inhibitor, second generation lecinoxoids, second generation naltrexone analog, seladelpar lysine, selatogrel, selvigaltin, semaglutide, SER-140, serelaxin, setanaxib, sevelamer hydrochloride, SFA-001, SFX-01, SGM-1019, SH-2442, SHC-023, SHC-028, Shiloah-1000, shortened GLP-1, SHR-2042, SHR-3167, SHR-3824, SIG-002, siplizumab, siRNA therapy, SIRT6 gene therapy, SIRT6 inhibitors, Sirtuin6 protein regulators, sitagliptin, sitagliptin+extended-release metformin, sitagliptin+glimepiride+metformin hydrochloride ER, sitagliptin+metformin, sitagliptin+metformin+pioglitazone, sitagliptin+simvastatin, sitagliptin fenilalanil hydrochloride, SK1-I, SL-100, SN-401, SN-406, SNP-610, SNP-630, soluble guanylate cyclase stimulator, sotagliflozin, SPL-891, SPN-0103-009, SPP-004, SQZ-TAC-TTD, SR-03, SR-040/041/042, SR-044, SRI-37330, SRT-015, ST-003, STM-003, STP-707, supalutai, survodutide, SVP insulin+SVP rapamycin, SVT-201, SY-004, SY-008, SY-009, SYHA-1805, SYN-020, SZN-043, T-1123, T-1123, T1D-ADV210, T2D-PT201, tacrolimus, targeted gene therapy, TB-001, TB59-2, TB-840, TB-D-004C, T-cell expressed protein, TCM-800B, TCR-TREGs, TDI-01, TE-8105, technetium Tc 99m tilmanocept, telazorlimab, temelimab, teneligliptin+metformin, teneligliptin hydrobromide, teneligliptin hydrobromide+canagliflozin hydrate, teplizumab, TERN-101, TERN-501, tesamorelin, testosterone prodrug, testosterone undecanoate, TG-68, TH-104, THDBH-101, THDBH-110/THDBH-111, THDBH-120/THDBH-121, thioacrylamide compounds, thyroid hormone receptor beta-selective agonists, tianagliflozin, TID-PT101, tilpisertib, timolumab, tipelukast, tirzepatide, tissue-specific immunomodulating bispecific antibodies, TIX-100, TJ-103, TJC-0316, TLC-065, TLC-1235, TLC-2716, TLC-3595, TLC-6740, TLY-004, TLY-012, tofogliflozin, TOL-3021, ToleraCell-001, ToleraCell-002, tolimidone, TP-352, TQ-05510, TQA-3526, TQ-F3083, Treg modulator therapy, trelagliptin succinate, TreXTAM, tricyclic pyrone compounds, TS-20004, TSG-03-117, TSL-0319, TT-01025, TTP-273, TTP-RA, TU-5113, tulinercept, TXR-611, TXR-612, TY-705, UBT-251, UI-068, ularitide, Ulinastatin, umbilical cord blood-derived mesenchymal stem cell therapy, umbilical cord-derived mesenchymal stem cells, UN-03, Uni-E4-Fc, UP-421, ursodeoxycholic acid, ursolic acid, UTAA-09, V-411, valsartan+celecoxib, VAR 200-03, VB-201, VB-601, VCT-220, VCTX-210, VCTX-212, VE-5708, VE-5773, vesicular monoamine transporter 2 antagonists, vidofludimus, vildagliptin, vildagliptin+metformin, vildagliptin+pioglitazone, vildagliptin+pioglitazone hydrochloride, vildagliptin SR+metformin SR, visepegenatide, VK-0612, VK-2735, VK-2735, oral, VK-2809, VNA-438, VN-B101, voglibose, voglibose+metformin, volagidemab, volixibat potassium ethanolate hydrate, vonafexor, vonifimod, VS-105, VUM-02, vutiglabridin, VX-264, VX-880, wanpagliflozin, WCDD-301, WS-012, WXSH-0038, WXSH-0078, WXSH-0213, XEN-103, XEN-D0501, xenin-fused peptides, XFB-19, XP-3924, XTYW-003, XW-013, XW-014, XW-015, XZP-5610, XZP-5695, XZP-6019, YA-6060, YD02-2022, YFQLXB-UC01, YG-1699, YH-2000, YH-25724, YH-40863, YHC-1102, YHC-1108, YHC-1131, YJH-0425, yogliptin, YW-1128, YYC-405, ZED-1227, zelasudil, zetomipzomib, zevaquenabant, ZG-0588, zibotentan, ziftomenib, Zituvio, ZMC-001, ZSP-0678, ZSP-1601, ZSYM-008, ZT-002, ZT-003, ZT-01, ZX-2010, ZX-2020, ZX-2021, or bi-specific antibodies targeting one or more targets referenced herein.

In some embodiments, a compound of the disclosure provided herein, or pharmaceutically acceptable salt thereof, is administered with one or more therapeutic agents selected from a PPARd inhibitor, GLP-1 agonist, IRAK4 inhibitor, TPL2 inhibitor, BTLA agonist, PD1 agonist, IL-12/23 p40 inhibitor, IL-23 inhibitor, or an FXR agonist.

In some embodiments, a compound of the disclosure provided herein, or pharmaceutically acceptable salt thereof, is administered with one or more therapeutic agents selected from seladelpar, edecesertib, tilpisertib fosmecarbil, GS-4571, GS-0272, GS-0151, or cilofexor.

### Synthesis of Intermediates

### Preparation of Intermediate I-1

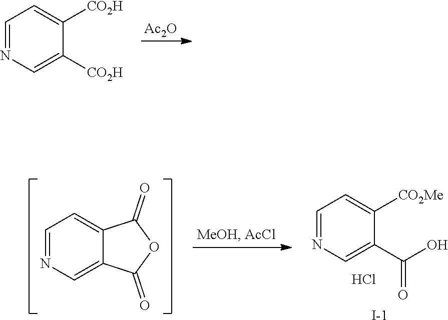

4-(methoxycarbonyl)nicotinic acid hydrochloride (I-1): pyridine-3,4-dicarboxylic acid (1.00 equiv, scaling factor) and acetic anhydride (2.2 equiv) were charged to a reactor. The resulting mixture was agitated at about 105 °C for about 4 hours and then cooled to about 10 °C. The mixture was transferred to a reactor containing premixed acetyl chloride (2.3 equiv) and methanol (4.7 volumes) and the contents were agitated at about 10 °C. After complete addition, the reactor contents were agitated at about 20 °C for about 4 hours. The slurry was centrifuged, washed with isopropanol (1.5 volumes), and dried under reduced pressure at about 45 °C for about 20 hours to afford I-1. ¹H NMR (400 MHz, DMSO-d₆) δ 9.03 (d, J=0.8 Hz, 1H), 8.89 (d, J=4.8 Hz, 1H), 7.66 (d, J=0.8 Hz, 1H), 4.98 (br s, 2H), 3.84 (s, 3H).

### Preparation of Intermediate 2

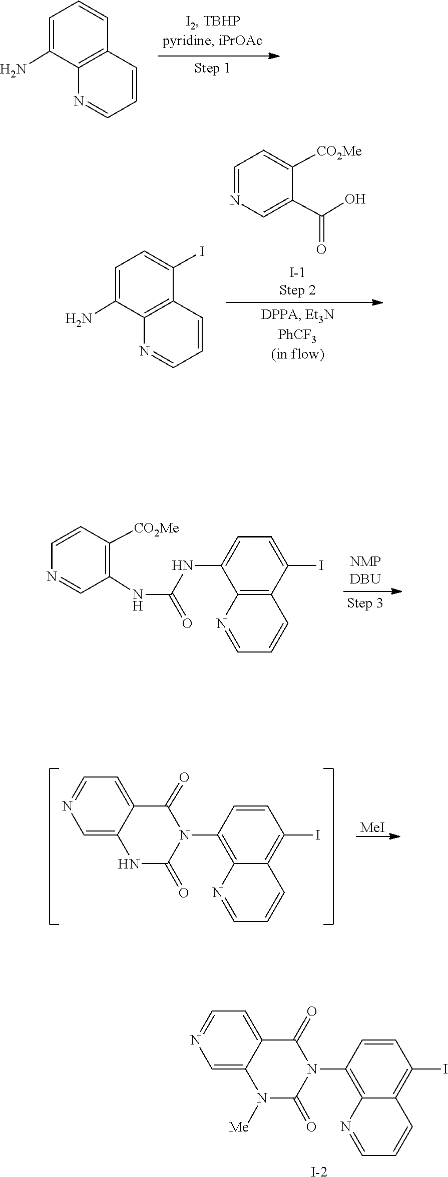

Step 1. Synthesis of 3-(5-iodoquinolin-8-yl)-1-methylpyrido[3,4-d]pyrimidine-2,4(1H,3H)-dione (I-2): quinolin-8-amine (1.00 equiv, scaling factor), iodine (0.6 equiv), pyridine (3.0 equiv), and isopropyl acetate (1.5 volume) were charged to a reactor. Reactor contents were agitated at about 25 °C for about 2 hours. The resulting mixture was adjusted to about 50 °C and charged with tert-butanol peroxide in water (0.55 equiv, 70% aqueous solution). The resulting mixture was agitated at about 50 °C for about 2 hours. Upon completion, the mixture was quenched with sodium thiosulfate (0.50 equiv) in water (1.0 volumes) while maintaining the internal temperature at about 50 °C. After agitating for about 1 hour, the mixture was charged with activated carbon (1.80 equiv) in water (0.5 volumes). The temperature was adjusted to about 25 °C and the stream was filtered through celite. The aqueous layer was extracted with isopropyl acetate (0.5 volumes) and the combined organics were washed with water (1.0 volumes), concentrated under reduced pressure, and charged with N-methyl-2-pyrrolidone (0.5 volumes). The reactor contents were concentrated under reduced pressure to about 5.5 volumes and agitated at about 20 °C. Reactor was charged with water (4.0 volumes) and the contents were agitated for about 2 hours. The slurry was filtered, washed with methanol (1.5 volumes) and water (3.5 volumes), and dried under reduced pressure at about 45 °C for about 20 hours to afford the titled compound. 1H NMR (400 MHz, DMSO-d6) δ 8.72 (dd, J=4.1, 1.5 Hz, 1H), 8.17 (dd, J=8.5, 1.5 Hz, 1H), 7.80 (d, J=8.1 Hz, 1H), 7.59 (dd, J=8.5, 4.1 Hz, 1H), 6.72 (d, J=8.1 Hz, 1H), 6.19 (s, 2H).

Step 2. Synthesis of Methyl 3-(3-(5-iodoquinolin-8-yl)ureido)isonicotinate: 5-iodoquinolin-8-amine (1.15 equiv), trifluorotoluene (6.0 volumes), I-1 (1.00 equiv, scaling factor), and triethylamine (2.50 equiv) were charged to a reactor and warmed to about 65 °C. The reactor contents were charged with a solution of diphenylphosphoryl azide (1.15 equiv) in trifluorotoluene (1.0 volumes) over 2 h while heating at about 65 °C. The resulting mixture was cooled to about 15 °C over about 8 h and filtered. The filter cake was rinsed with trifluorotoluene (1.0 volume), then twice with MTBE (1.0 volume per rinse) and dried under reduced pressure at about 40 °C for about 20 hours to afford the titled compound. ¹H NMR (400 MHz, DMSO-d₆) δ 10.25 (s, 1H), 10.09 (s, 1H), 9.02 (s, 2H), 8.51 (dd, J=8.5, 1.4 Hz, 1H), 8.45 (d, J=8.5 Hz, 1H), 8.40 (d, J=5.0 Hz, 1H), 7.91 (d, J=8.5 Hz, 1H), 7.80 (dd, J=8.5, 4.2 Hz, 1H), 7.65

Step 3. Synthesis of 3-(5-iodoquinolin-8-yl)-1-methylpyrido[3,4-d]pyrimidine-2,4(1H,3H)-dione (I-2): Methyl 3-(3-(5-iodoquinolin-8-yl)ureido)isonicotinate (1.00 equiv, scaling factor), 1,8-diazabicyclo[5.4.0]undec-7-ene (1.00 equiv), and N-methyl-2-pyrrolidone (6.0 volumes) were charged to a reactor. The reactor contents were agitated at about 20 °C for about 3 hours. The reactor contents were adjusted to about 0 °C and charged with iodomethane (1.10 equiv) and N-methyl-2-pyrrolidone (1.0 volumes) while maintaining the internal temperature at about 5 °C. After complete addition, the resulting mixture was agitated at about 20 °C for about 16 hours. The reactor was then charged with water (3.5 volumes) while maintaining the internal temperature at about 20 °C and agitated for about 16 hours. The slurry was centrifuged and the cake was washed with N,N-dimethylformamide (1.0 volumes), water (0.5 volumes), isopropyl alcohol (1.5 volumes), and tert-butyl methyl ether (1.5 volumes). The cake was dried under pressure at about 50 °C for about 30 hours to afford title compound. ¹H NMR (400 MHz, DMSO-d₆) δ 9.05 (d, J=0.7 Hz, 1H), 8.84 (dd, J=4.2, 1.6 Hz, 1H), 8.60 (d, J=5.0 Hz, 1H), 8.46 (dd, J=8.6, 1.5 Hz, 1H), 8.40 (d, J=7.8 Hz, 1H), 7.92 (dd, J=5.0, 0.8 Hz, 1H), 7.72 (dd, J=8.6, 4.2 Hz, 1H), 7.66 (d, J=7.8 Hz, 1H), 3.65 (s, 3H).

### Preparation of Intermediate 3

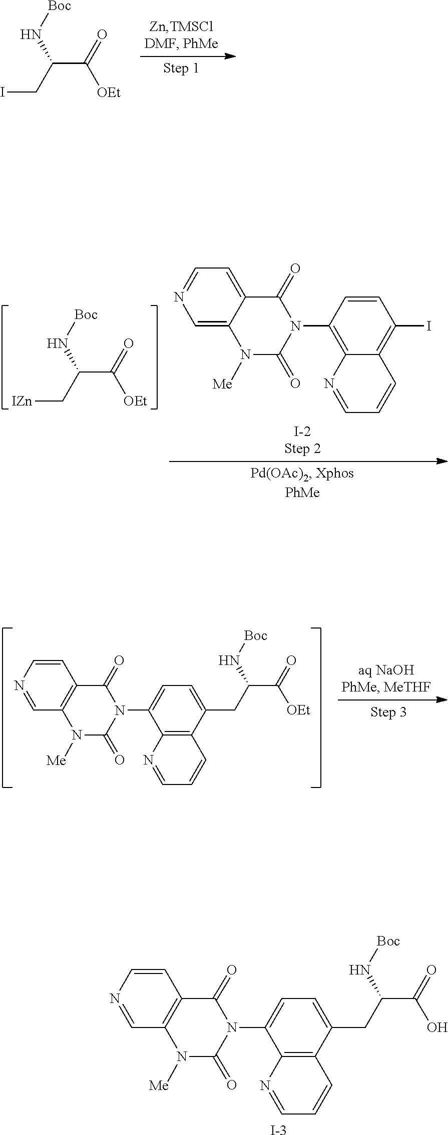

Step 1. Synthesis of (R)-(2-((tert-butoxycarbonyl)amino)-3-methoxy-3-oxopropyl)zinc(II) iodide: ethyl (R)-2-((tert-butoxycarbonyl)amino)-3-iodopropanoate (1.0 equiv, scaling factor) charged in two portions. A reactor with ethyl (R)-2-((tert-butoxycarbonyl)amino)-3-iodopropanoate (0.10 equiv), N,N-dimethylformamide (1.0 volumes), toluene (3.0 volumes), and zinc (1.4 equiv) was charged with trimethylsilyl chloride (0.03 equiv) and aged at about 20 °C for about 1 hour. A solution of methyl (R)-2-((tert-butoxycarbonyl)amino)-3-iodopropanoate (0.90 equiv) in toluene (3.0 volumes) was added over about 7 hours. After complete addition, the resulting mixture of organozinc (R)-(2-((tert-butoxycarbonyl)amino)-3-methoxy-3-oxopropyl)zinc(II) iodide was aged for about 3 hours and then cooled to about −15 °C until further use.

Step 2. Synthesis of methyl (2S)-2-((tert-butoxycarbonyl)amino)-3-(8-(1-methyl-2,4-dioxo-1,4-dihydropyrido[3,4-d]pyrimidin-3(2H)-yl)quinolin-5-yl)propanoate: I-2 (1.00 equiv, scaling factor), palladium(II) acetate (0.025 equiv), 2-dicyclohexylphosphino-2′,4′,6′-diisopropylbiphenyl (0.044 equiv), and toluene (7.0 volumes) were charged to a glass-lined reactor and the contents were agitated at about 20 °C. The reactor was then charged with organozinc (R)-(2-((tert-butoxycarbonyl)amino)-3-methoxy-3-oxopropyl)zinc(II) iodide solution (0.13 equiv) and the mixture was agitated at about 60 °C for about 2 hours and then charged with additional organozinc (R)-(2-((tert-butoxycarbonyl)amino)-3-methoxy-3-oxopropyl)zinc(II) iodide solution (1.17 equiv) over about 3 hours. After complete addition, the resulting mixture was agitated at about 60 °C for about 3 hours. Next, the reactor contents were cooled to about 35 °C, charged with Celite (0.35 weight ratio) and aqueous 17.5% ammonium chloride solution (7.0 volumes), and aged for about 1 hour. The resulting slurry was cooled to about 20 °C, filtered, and the cake was washed with toluene (1.0 volumes). The combined filtrate was transferred to a clean glass-lined reactor and the aqueous layer was discharged. The organics were washed with aqueous 17.5% ammonium chloride solution (7.0 volumes), the phases were separated, and the organic layer was washed with an aqueous solution of N-acetyl-L-cysteine (0.50 equiv) and potassium carbonate (0.86 equiv) in water (10 volumes) at about 40 °C for about 7 hours. The reactor contents were cooled to about 20 °C, the aqueous layer was discharged, and the organics were washed with water (5.0 volumes) to provide a solution of the title compound in toluene.

Step 3. Synthesis of (2S)-2-((tert-butoxycarbonyl)amino)-3-(8-(1-methyl-2,4-dioxo-1,4-dihydropyrido[3,4-d]pyrimidin-3(2H)-yl)quinolin-5-yl)propanoic acid (I-3): The solution of Methyl (2S)-2-((tert-butoxycarbonyl)amino)-3-(8-(1-methyl-2,4-dioxo-1,4-dihydropyrido[3,4-d]pyrimidin-3(2H)-yl)quinolin-5-yl)propanoate stream in toluene was charged to a reactor. The contents were adjusted to about 55 °C and reduced under pressure to about 6.0 volumes. The reactor was charged with 2-methyltetrahydrofuran (3.9 volumes) and the resulting slurry was filtered, collecting the filtrate in a reactor. The reactor contents were charged with 2-methyltetrahydrofuran (1.8 volumes) and the contents were adjusted to about 5 °C. Next, the reactor was charged with sodium hydroxide (2.0 equiv) in water (7.4 volumes) and the contents were agitated at about 5 °C for about 6 hours. The aqueous layer was discharged, and the reactor contents were charged with 2-methyltetrahydrofuran (3.2 volumes) and aqueous 7% formic acid solution (1.6 volumes). The reactor contents were adjusted to about 45 °C and charged with additional aqueous 7% formic acid solution (1.6 volumes) over 4 hours. The resulting mixture was cooled to about 5 °C and agitated for about 12 hours. The slurry was filtered, and the cake was washed with water (8.0 volumes). The cake was then slurried in toluene (27.0 volumes) and ethanol (3.0 volumes), filtered, and dried at about 70 °C under reduced pressure for about 30 hours to afford title compound. ¹H NMR (400 MHz, DMSO-d₆) δ 12.85 (s, 1H), 9.04 (s, 1H), 8.82 (d, J=4.1 Hz, 1H), 8.60 (t, J=7.3 Hz, 2H), 7.91 (dd, J=5.0, 2.1 Hz, 1H), 7.75 (d, J=7.5 Hz, 1H), 7.65 (dd, J=8.7, 3.7 Hz, 2H), 7.30 (d, J=8.3 Hz, 1H), 4.33-4.15 (m, 1H), 3.65 (d, J=1.7 Hz, 4H), 3.42-3.35 (m, 1H), 1.32 (s, 9H).

### Preparation of Intermediate 4

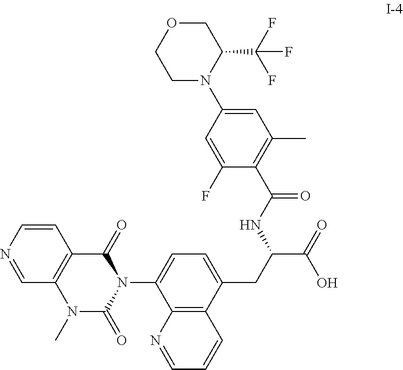

Intermediate 4 (I-4): was synthesized according to know methods, such as, those disclosed in U.S. Pat. No. 11,116,760, the contents of which are incorporated herein by reference in their entirety.

### Preparation of Intermediate 5

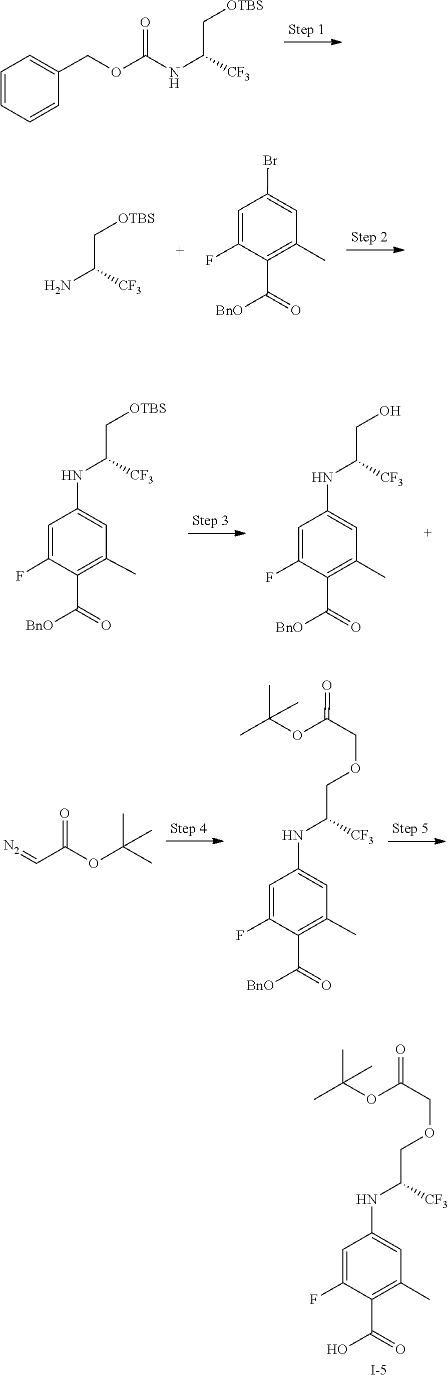

Step 1. Synthesis of (2R)-3-[tert-butyl(dimethyl)silyl]oxy-1,1,1-trifluoro-propan-2-amine: benzyl N-[(1R)-1-[[tert-butyl(dimethyl)silyl]oxymethyl]-2,2,2-trifluoro-ethyl]carbamate (270 mg, 0.69 mmol) and 10% Pd/C (wet, 73 mg, 0.07 mmol) were suspended in EtOH (6.0 mL). The headspace was then evacuated/backfilled with dry N₂ three times followed by H₂ gas. The reaction was stirred at ambient temperature under a balloon of H₂ for about 45 minutes at which point the catalyst was filtered off and washed with additional EtOH. The filtrate was evaporated to provide the title compound which was used without purification. ES/MS (m/z) 244.1 (M+H)+.

Step 2. Synthesis of benzyl 4-[[(1R)-1-[[tert-butyl(dimethyl)silyl]oxymethyl]-2,2,2-trifluoro-ethyl]amino]-2-fluoro-6-methyl-benzoate: To a vial were charged (2R)-3-[tert-butyl(dimethyl)silyl]oxy-1,1,1-trifluoro-propan-2-amine (0.69 mmol), benzyl 4-bromo-2-fluoro-6-methyl-benzoate (250 mg, 0.76 mmol), XPhos Pd Gen 4 (59 mg, 0.07 mmol), and Cs₂CO₃ (560 mg, 1.7 mmol). The vial was flushed with dry N₂ and then dioxane (5.5 mL) was added. The mixture was heated to about 110 °C for about 20 hours at which point it was cooled, diluted with EtOAc, and filtered through a pad of Celite. The filtrate was then evaporated to provide crude material which was purified via flash chromatography (100% hex→60% EtOAc/Hex) to afford title compound. ES/MS (m/z) 486.1 (M+H)+.

Step 3. Synthesis of benzyl 2-fluoro-6-methyl-4-[[(1R)-2,2,2-trifluoro-1-(hydroxymethyl)ethyl]amino]benzoate: benzyl 4-[[(1R)-1-[[tert-butyl(dimethyl)silyl]oxymethyl]-2,2,2-trifluoro-ethyl]amino]-2-fluoro-6-methyl-benzoate (110 mg, 0.22 mmol) was flushed with dry N₂, dissolved in THF (5.0 mL), and the solution stirred at ambient temp. TBAF (1.0 M, 0.44 mL) was then added and the mixture stirred for about 1 hour. At this point, water and 10% aq. KHSO₄ were added, and the mixture extracted 3× with EtOAc. The combined organic extracts were washed with brine, dried over MgSO₄, filtered, and evaporated. The crude residue was purified via flash chromatography (100% hex→80% EtOAc/Hex) to afford title compound. ES/MS (m/z) 372.0 (M+H)+.

Step 4. Synthesis of benzyl 4-[[(1R)-1-[(2-tert-butoxy-2-oxo-ethoxy)methyl]-2,2,2-trifluoro-ethyl]amino]-2-fluoro-6-methyl-benzoate: A vial was charged with benzyl 2-fluoro-6-methyl-4-[[(1R)-2,2,2-trifluoro-1-(hydroxymethyl)ethyl]amino]benzoate (82 mg, 0.21 mmol) and Rh₂(OAc)₄ (9.3 mg, 0.02 mmol) and purged with dry N₂. DCM (1.5 mL) was added and the mixture stirred in an ice water bath. tert-Butyl Diazoacetate (35 μL, 0.25 mmol) was added and the reaction maintained in the cooling bath. After about 1 hour, the mixture was loaded directly onto a silica gel loading cartridge and purified via flash chromatography (100% hex→60% EtOAc/Hex) to afford title compound. ES/MS (m/z) 486.1 (M+H)+.

Step 5. Synthesis of 4-[[(1R)-1-[(2-tert-butoxy-2-oxo-ethoxy)methyl]-2,2,2-trifluoro-ethyl]amino]-2-fluoro-6-methyl-benzoic acid (I-5): A vial was charged with benzyl 4-[[(1R)-1-[(2-tert-butoxy-2-oxo-ethoxy)methyl]-2,2,2-trifluoro-ethyl]amino]-2-fluoro-6-methyl-benzoate (67 mg, 0.14 mmol), 10% Pd/C (wet, 15 mg, 0.01 mmol), and EtOH (1.5 mL). The mixture was then bubbled with dry N₂ followed by H₂. The reaction was stirred for about 1 hour at ambient temperature under a balloon of H₂ at which time the catalyst was filtered off. The filtrate was evaporated to provide title compound which was used without purification. ES/MS (m/z) 396.0 (M+H)+.

### EXAMPLES

### Methods

Compounds of Formula I were synthesized according to know methods, such as, those disclosed in U.S. Pat. No. 11,116,760, the contents of which are incorporated herein by reference in their entirety.

### X-Ray Powder Diffraction (XRPD)

Some XRPD patterns were collected with a an PANanalytical XPERT-PRO MPD diffractometer using an incident beam of Cu Ku radiation produced using a long, fine-focus source and a nickel filter. The diffractometer was configured using the symmetric Bragg-Brentano geometry. Prior to the analysis, a silicon specimen (NIST SRM 640e, Malvern Panalytical Inc., Westborough, Massachusetts, USA) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcel1D-Medipix3 PASS (programmable anti-scatter slit), located 240 mm from the sample and Data Collector software v. 2.2b.

Other XRPD patterns were collected with a PANalytical Empyrean diffractometer using an incident beam of Cu Ku radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcel1D-Medipix3 PASS (programmable anti-scatter slit), located 240 mm from the sample and Data Collector software v. 7.2b.

### Differential Scanning Calorimetry (DSC)

Differential Scanning Calorimetry (DSC) data were collected using a TA Instruments Q2000 differential scanning calorimeter. Temperature calibration was performed using NIST-traceable indium metal. The sample was placed into a T zero aluminum DSC pan, covered with a lid pierced using a needle. The weight was then accurately recorded. A weighed aluminum pan configured as the sample pan was placed on the reference side of the cell. The sample was heated from 20 °C to 300 °C at 10 °C/minute.

### Thermo-Gravimetric Analysis

Thermogravimetric Analysis (TGA) data were collected using a TA Instruments Q5000 thermogravimetric analyzer. Temperature calibration was performed using nickel and Alumel™. Each sample was placed in an aluminum pan and inserted into the TG furnace. The furnace was heated under a nitrogen purge. The sample was heated from ambient to 300 °C at 10 °C/minute.

### Compounds of Formula I

### Formula I Freebase Form I

Formula I freebase Form I was prepared from amorphous Formula I freebase with chemical purity greater than 98% assay by weight. About 50 mg of the amorphous solid was suspended in 0.25 mL of ethanol. A slurry was obtained after one day of stirring at about 20 °C. The solids were isolated by centrifugation and dried in the vacuum oven at about 50 °C for about 3 days.

Formula I freebase Form I was also obtained using about 50 mg of amorphous GS-1427 freebase in about 0.4 mL of several other organic solvents, including acetonitrile, methanol, acetone, methyl ethyl ketone, methyl isobutyl ketone, tetrahydrofuran, 2-methyl tetrahydrofuran, ethyl acetate, isopropyl acetate, and toluene. In some cases, seeding of the amorphous solids with small amount of GS-1427 freebase Form I crystals was used.

XRPD analysis was conducted. FIG. 1 shows and XRPD pattern of the Formula I freebase Form I compound. XRPD peaks were identified and are included in Table 1 below.

TABLE 1

| Pos. [°2Th.] | Rel. Int. [%] |
|---|---|
| 6.4 | 12 |
| 8.5 | 5 |
| 9.5 | 85 |
| 9.8 | 31 |
| 10.5 | 49 |
| 11.1 | 40 |
| 11.9 | 8 |
| 12.8 | 6 |
| 13.8 | 48 |
| 14.0 | 66 |
| 14.5 | 28 |
| 16.3 | 36 |
| 16.7 | 99 |
| 16.9 | 46 |
| 18.0 | 33 |
| 18.9 | 40 |
| 19.1 | 14 |
| 19.5 | 37 |
| 19.6 | 35 |
| 20.4 | 19 |
| 21.0 | 100 |
| 22.0 | 55 |
| 22.3 | 14 |
| 23.3 | 44 |
| 24.2 | 12 |
| 25.2 | 11 |
| 25.7 | 61 |
| 26.2 | 18 |
| 27.5 | 24 |
| 28.8 | 18 |
| 29.1 | 19 |
| 30.6 | 6 |
| 31.7 | 12 |
| 32.3 | 11 |
| 32.9 | 13 |
| 33.3 | 9 |
| 34.0 | 7 |
| 35.3 | 8 |
| 36.4 | 7 |
| 37.6 | 8 |
| 38.3 | 6 |
| 39.0 | 12 |

A diffractogram, was indexed and the unit cell dimensions were calculated. Results are reported in Table 2 below. Data collection was performed on a Rigaku SuperNova diffractometer, equipped with a copper anode microfocus sealed X-ray tube (Cu Ka 1=1.54184 Å) and a Dectris Pilatus3 R 200K hybrid pixel array detector. Cell constants and an orientation matrix for data collection were obtained from least-squares refinement using the setting angles of 11405 reflections in the range 4.2540°<q<75.5640°. The space group was determined by the program CRYSALISPRO [CrysAlisPro 1.171.41.93a (Rigaku Oxford Diffraction, 2020)] to be P2₁2₁2₁ (international tables no. 19).

The data were collected to a maximum diffraction angle (2θ) of 151.704° at room temperature.

TABLE 2

| Space group | P2₁2₁2₁ | Volume (Å³) | 3410.22(7) |
|---|---|---|---|
| Unit cell parameters | | | |
| a (Å) | 8.85800(10) | α (°) | =90 |
| b (Å) | 18.5742(2) | β (°) | =90 |
| c (Å) | 20.7270(3) | γ (°) | =90 |

Single Crystal X-ray Crystallography for freebase Form I was conducted, and the data showed the crystal system for Form I is anhydrous.

DSC analysis was performed. FIG. 2 shows a DSC thermogram of the compound of Formula I freebase Form 1. The DSC analysis revealed an endothermic transition with onset at about 177 °C.

TGA analysis was performed. FIG. 3 shows a TGA thermogram of the compound of Formula I freebase Form I. The TGA analysis indicates that the phase loses about 0.6% of its weight before melting.

### Formula I Freebase Acetophenone Solvate

Formula I freebase acetophenone solvate was prepared by stirring Formula I amorphous freebase in acetophenone. Heptane was added. The wet solids were isolated and dried in the vacuum oven at 50 °C.

XRPD analysis was conducted. FIG. 4 shows and XRPD pattern of the Formula I freebase acetophenone solvate compound. XRPD peaks were identified and are included in Table 3 below.

TABLE 3

| Pos. [°2Th.] | Rel. Int. [%] |
|---|---|
| 3.8 | 11 |
| 8.3 | 22 |
| 11.5 | 100 |
| 11.9 | 64 |
| 12.0 | 24 |
| 13.2 | 18 |
| 14.2 | 6 |
| 14.7 | 13 |
| 15.3 | 43 |
| 16.4 | 33 |
| 16.6 | 20 |
| 17.4 | 7 |
| 18.0 | 13 |
| 18.3 | 24 |
| 19.2 | 18 |
| 19.5 | 10 |
| 20.0 | 13 |
| 20.5 | 9 |
| 21.0 | 18 |
| 21.3 | 15 |
| 21.7 | 6 |
| 22.1 | 7 |
| 22.6 | 9 |
| 23.6 | 7 |
| 24.5 | 14 |
| 25.1 | 8 |
| 25.7 | 12 |
| 26.3 | 14 |
| 26.9 | 7 |
| 27.6 | 9 |
| 29.1 | 9 |
| 29.6 | 6 |
| 30.9 | 10 |
| 32.5 | 11 |
| 33.5 | 7 |

DSC analysis was performed. FIG. 5 shows a DSC thermogram of the compound of Formula I freebase acetophenone solvate. The DSC analysis revealed an endothermic transition with onset at about 164 °C.

TGA analysis was performed. FIG. 6 shows a TGA thermogram of the compound of Formula I acetophenone solvate. The TGA analysis indicates that the phase loses about 4.9% of its weight during the melt.

### Formula I Freebase Anisole Solvate

Formula I freebase anisole solvate was prepared by stirring Formula I amorphous freebase in anisole. An equal amount of methyl-t-butyl ether to the amount of anisole was added. The mixture was seeded with a small amount of Formula I freebase acetophenone solvate. The wet solids were isolated and dried in the vacuum oven at 50 °C.

XRPD analysis was conducted. FIG. 7 shows and XRPD pattern of the Formula I freebase anisole solvate compound. XRPD peaks were identified and are included in Table 4 below.

TABLE 4

| Pos. [°2Th.] | Rel. Int. [ % ] |
|---|---|
| 3.8 | 4 |
| 8.3 | 8 |
| 8.7 | 6 |
| 11.5 | 100 |
| 11.9 | 85 |
| 13.2 | 12 |
| 13.6 | 10 |
| 14.7 | 9 |
| 15.4 | 42 |
| 16.8 | 32 |
| 17.6 | 16 |
| 18.3 | 35 |
| 19.2 | 25 |
| 20.1 | 12 |
| 20.7 | 19 |
| 21.4 | 26 |
| 23.6 | 13 |
| 23.7 | 9 |
| 24.6 | 24 |
| 25.2 | 19 |
| 25.8 | 26 |
| 26.3 | 26 |
| 27.0 | 13 |
| 27.9 | 15 |
| 29.2 | 14 |
| 30.1 | 9 |
| 30.9 | 13 |
| 32.5 | 12 |
| 34.5 | 7 |
| 35.9 | 8 |
| 38.9 | 8 |

DSC analysis was performed. FIG. 8 shows a DSC thermogram of the compound of Formula I freebase anisole solvate. The DSC analysis revealed an endothermic transition with onset at about 157 °C.

TGA analysis was performed. FIG. 9 shows a TGA thermogram of the compound of Formula I anisole solvate. The TGA analysis indicates that the phase loses about 0.6% of its weight before the melt and about 4.7% during the melt.

### Formula I Gentisate Form I

Formula I gentisate Form I was prepared by stirring about 100 mg of amorphous Formula I freebase in 0.6 mL of acetonitrile. About one molar equivalent of gentisic acid was added, and the mixture was stirred at about 20 °C for about 3 days. A slurry was obtained. The solids were isolated by centrifugation and air-dried for about a day.

Formula I gentisate Form I can also be prepared using the same procedure with 1.5 equivalents of gentisic acid.

XRPD analysis was conducted. FIG. 10 shows and XRPD pattern of the Formula I gentisate Form I compound. XRPD peaks were identified and are included in Table 5 below.

TABLE 5

| Pos. [°2Th.] | Rel. Int. [ % ] |
|---|---|
| 4.8 | 26 |
| 5.9 | 100 |
| 9.1 | 13 |
| 9.6 | 15 |
| 10.8 | 8 |
| 11.7 | 35 |
| 13.3 | 13 |
| 13.8 | 44 |
| 15.3 | 7 |
| 16.4 | 49 |
| 17.1 | 18 |
| 17.9 | 16 |
| 18.2 | 11 |
| 19.1 | 3 |
| 20.6 | 15 |
| 21.8 | 19 |
| 24.0 | 12 |
| 25.0 | 17 |
| 25.6 | 16 |
| 27.4 | 8 |
| 28.8 | 5 |
| 30.0 | 8 |
| 30.8 | 4 |

DSC analysis was performed. FIG. 11 shows a DSC thermogram of the compound of Formula I freebase gentistate solvate. The DSC analysis revealed an endothermic transition with onset at about 147 °C. This transition is preceded by a broad endothermic event which is likely due to solvent loss.

TGA analysis was performed. FIG. 12 shows a TGA thermogram of the compound of Formula I gentisate solvate. The TGA analysis indicates that the phase loses about 4.1% of its weight before the endothermic event at 147 °C.

### Formula I Mono-Malonate Monohydrate Acetonitrile Solvate

Formula I mono-malonate monohydrate acetonitrile solvate was prepared by dissolving about 500 mg of amorphous Formula I freebase in 4 mL of acetonitrile. About one molar equivalent of malonic acid was added. A slurry was obtained. The mixture was stirred at about 20 °C for two days. The solids were isolated by vacuum filtration and the wet solids were dried in the vacuum oven at about 50 °C for about 3 days.

Formula I mono-malonate monohydrate acetonitrile solvate was also prepared by adding about 1.2 equivalents of malonic acid to a solution of Formula I freebase in a mixture of acetonitrile (4.5 volumes), methyl-t-butyl ether (4.5 volumes) and water (0.07 volumes).

Formula I mono-malonate monohydrate acetonitrile solvate was also prepared by reslurrying about 40 mg of Formula I bis-malonate in about 0.3 mL of acetonitrile for about 2 days.

In the above two preparations, Formula I mono-malonate monohydrate acetonitrile solvate can be isolated by deliquoring the solids by centrifugation or vacuum filtration to obtain a wet solid. Formula I mono-malonate monohydrate acetonitrile solvate can also be obtained by air-drying the wet solids or drying the wet solid in a vacuum oven, until the residual acetonitrile content in the solids is greater than or approximately 5% by weight.

XRPD analysis was conducted. FIG. 13 shows and XRPD pattern of the Formula I mono-malonate monohydrate acetonitrile Form I compound. XRPD peaks were identified and are included in Table 6 below.

TABLE 6

| Pos. [°2Th.] | Rel. Int. [ % ] |
|---|---|
| 5.4 | 5 |
| 7.5 | 100 |
| 7.8 | 48 |
| 11.5 | 22 |
| 12.1 | 24 |
| 13.2 | 19 |
| 13.8 | 41 |
| 15.7 | 31 |
| 16.0 | 41 |
| 16.2 | 33 |
| 17.5 | 32 |
| 17.7 | 28 |
| 18.6 | 11 |
| 19.0 | 20 |
| 19.2 | 31 |
| 19.5 | 27 |
| 19.6 | 25 |
| 20.2 | 24 |
| 20.5 | 11 |
| 21.1 | 37 |
| 22.1 | 22 |
| 22.3 | 25 |
| 22.7 | 11 |
| 23.2 | 21 |
| 23.8 | 10 |
| 24.9 | 54 |
| 27.3 | 8 |
| 28.8 | 4 |
| 29.2 | 4 |
| 29.9 | 9 |
| 30.1 | 11 |
| 32.7 | 8 |
| 35.6 | 4 |
| 36.7 | 4 |
| 37.8 | 2 |

A diffractogram, was indexed and the unit cell dimensions were calculated. Results are reported in Table 7 below. Data collection was performed on a Rigaku SuperNova diffractometer, equipped with a copper anode microfocus sealed X-ray tube (Cu Ka 1=1.54184 Å) and a Dectris Pilatus3 R 200K hybrid pixel array detector. Cell constants and an orientation matrix for data collection were obtained from least-squares refinement using the setting angles of 11386 reflections in the range 3.9290°<q<75.2020°. The space group was determined by the program CRYSALISPRO [CrysAlisPro 1.171.41.93a (Rigaku Oxford Diffraction, 2020)] to be P2₁2₁2₁ (international tables no. 19).

The data were collected to a maximum diffraction angle (2θ) of 151.564° at room temperature.

TABLE 7

| Space group | P2₁2₁2₁ | Volume (Å³) | 4314.21(11) |
|---|---|---|---|
| Unit cell parameters | | | |
| a (Å) | 8.18250(10) | α (°) | =90 |
| b (Å) | 22.4846(4) | β (°) | =90 |
| c (Å) | 23.4493(3) | γ (°) | =90 |

Single Crystal X-ray Crystallography for Formula I mono-malonate monohydrate acetonitrile solvate was conducted, and the data confirms the stoichiometry and solvation state.

DSC analysis was performed. FIG. 14 shows a DSC thermogram of the compound of Formula I mono-malonate monohydrate acetonitrile solvate. The DSC analysis revealed an endothermic transition with onset at about 80 °C and a second endothermic transition with an onset at about 126 °C.

TGA analysis was performed. FIG. 15 shows a TGA thermogram of the compound of Formula I mono-malonate monohydrate acetonitrile solvate. The TGA analysis indicates that the phase loses about 5% of its weight before 126 °C and a 13% weight loss between 126 °C and 250 °C.

### Formula I Mono-Malonate Form I

Formula I mono-malonate Form I was prepared by dissolving about 500 mg of amorphous Formula I freebase in 4 mL of acetonitrile. About one molar equivalent of malonic acid was added. A slurry was obtained. The mixture was stirred at about 20 °C for two days. The solids were isolated by vacuum filtration and the wet solids were dried in the vacuum oven at about 50 °C for about 8 days.

Formula I mono-malonate Form I can also be prepared by drying Formula I mono-malonate monohydrate acetonitrile solvate in the vacuum oven at 50 °C until all the solvent has been removed.

Formula I mono-malonate Form I can also be prepared by drying solvates prepared from the following solvents: acetonitrile, acetone, methyl ethyl ketone, methylene chloride, tetrahydrofuran and 2-methyltetrahydrofuran.

XRPD analysis was conducted. FIG. 16 shows and XRPD pattern of the Formula I mono-malonate Form I compound. XRPD peaks were identified and are included in Table 8 below.

TABLE 8

| Pos. [°2Th.] | Rel. Int. [ % ] |
|---|---|
| 5.6 | 43 |
| 8.1 | 100 |
| 8.9 | 15 |
| 12.4 | 50 |
| 13.6 | 5 |
| 14.3 | 24 |
| 15.9 | 12 |
| 16.3 | 37 |
| 16.7 | 57 |
| 17.0 | 79 |
| 17.8 | 10 |
| 18.2 | 36 |
| 19.7 | 17 |
| 20.0 | 39 |
| 21.4 | 7 |
| 22.7 | 15 |
| 23.1 | 27 |
| 23.8 | 36 |
| 24.1 | 35 |
| 25.1 | 8 |
| 25.8 | 12 |
| 26.6 | 18 |
| 28.1 | 4 |
| 28.9 | 4 |
| 30.2 | 4 |
| 34.2 | 3 |

DSC analysis was performed. FIG. 17 shows a DSC thermogram of the compound of Formula I mono-malonate Form I. The DSC analysis revealed an endothermic transition with onset at about 125 °C.

TGA analysis was performed. FIG. 18 shows a TGA thermogram of the compound of Formula I mono-malonate Form I. The TGA analysis indicates that the phase loses about 0.3% of its weight before 110 °C.

### Formula I Mono-Malonate Form II

Formula I mono-malonate Form II was prepared by suspending about 40 mg of Formula I amorphous freebase in 0.4 mL of acetonitrile. Approximately one molar equivalent of malonic acid was added to the resulting solution and the mixture was stirred at ambient temperature for about 11 days. A slightly cloudy solution was obtained. The acetonitrile was removed by rotary evaporation and 0.4 mL of methanol was added. No slurry was obtained. The methanol was removed by rotary evaporation and 0.3 mL of ethyl acetate was added. After about one day a slurry was obtained. The solids were isolated by centrifugation and dried in a vacuum oven at about 50 °C for about a day.

XRPD analysis was conducted. FIG. 19 shows and XRPD pattern of the Formula I mono-malonate Form II compound. XRPD peaks were identified and are included in Table 9 below.

TABLE 9

| Pos. [°2Th.] | Rel. Int. [ % ] |
|---|---|
| 7.9 | 37 |
| 11.6 | 59 |
| 12.5 | 100 |
| 13.1 | 46 |
| 14.6 | 46 |
| 15.5 | 44 |
| 16.2 | 40 |
| 17.4 | 39 |
| 19.4 | 12 |
| 20.0 | 6 |
| 21.7 | 12 |
| 23.0 | 28 |
| 25.0 | 25 |
| 25.9 | 25 |
| 27.4 | 19 |

DSC analysis was performed. FIG. 20 shows a DSC thermogram of the compound of Formula I mono-malonate Form II. The DSC analysis revealed an endothermic transition with onset at about 128 °C.

TGA analysis was performed. FIG. 21 shows a TGA thermogram of the compound of Formula I mono-malonate Form II. The TGA analysis indicates that the phase loses about 0.7% before degrading.

### Formula I Mono-Malonate Acetone Solvate

Formula I mono-malonate acetone solvate was prepared by dissolving about 10 gr of amorphous Formula I freebase in 40 mL of acetone. About 6 mL of water was added, resulting in homogeneous solution with agitation. About one molar equivalent of malonic acid was added as a solution of malonic acid in acetone. A slurry was obtained. About 34 mL of water were charged into the reactor and the contents were agitated for about a day. The slurry was sampled, and centrifuge filtered to obtain GS-1427 mono-malonate acetone solvate.

The bulk slurry was then filtered under vacuum and the filter cake was washed with a mixture of acetone and water. The wet solids were dried in the vacuum oven at about 40 °C for about 7 days to obtain GS-1427 mono-malonate Form I.

XRPD analysis was conducted. FIG. 22 shows and XRPD pattern of the Formula I mono-malonate acetone solvate compound. XRPD peaks were identified and are included in Table 10 below.

TABLE 10

| Pos. [°2Th.] | Rel. Int. [ % ] |
|---|---|
| 5.4 | 2 |
| 7.5 | 15 |
| 7.8 | 73 |
| 11.5 | 2 |
| 12.1 | 2 |
| 12.3 | 3 |
| 13.8 | 4 |
| 13.9 | 6 |
| 15.6 | 100 |
| 16.0 | 10 |
| 17.5 | 7 |
| 17.7 | 8 |
| 18.5 | 2 |
| 19.1 | 11 |
| 19.4 | 15 |
| 19.9 | 5 |
| 20.4 | 4 |
| 21.0 | 97 |
| 21.7 | 7 |
| 21.9 | 5 |
| 22.2 | 15 |
| 22.6 | 9 |
| 23.1 | 6 |
| 23.6 | 7 |
| 23.8 | 12 |
| 24.3 | 4 |
| 24.7 | 66 |
| 26.2 | 2 |
| 27.1 | 4 |
| 28.6 | 26 |
| 29.4 | 3 |
| 29.6 | 6 |
| 29.9 | 14 |
| 30.6 | 4 |
| 31.6 | 6 |
| 32.4 | 25 |
| 33.4 | 6 |
| 34.3 | 3 |
| 35.3 | 4 |
| 36.4 | 13 |
| 36.8 | 4 |
| 37.5 | 3 |
| 39.1 | 7 |

### Formula I Mono-Malonate 2-Methyltetrahydrofuran Solvate

Formula I mono-malonate 2-methyltetrahydrofuran solvate was prepared by adding around 0.4 mL of 2-methyltetrahydrofuran to about 50 mg of Formula I mono-malonate Form II. The mixture was stirred at ambient for about 19 days and the slurry was filtered with centrifugation to obtain Formula I mono-malonate 2-methyltetrahydrofuran solvate.

XRPD analysis was conducted. FIG. 23 shows an XRPD pattern of the Formula I mono-malonate 2-methyltetrahydrofuran solvate compound. XRPD peaks were identified and are included in Table 11 below.

TABLE 11

| Pos. [°2Th.] | Rel. Int. [ % ] |
|---|---|
| 5.4 | 8 |
| 7.4 | 96 |
| 7.7 | 19 |
| 8.3 | 7 |
| 11.3 | 16 |
| 12.0 | 20 |
| 13.0 | 10 |
| 13.6 | 55 |
| 14.8 | 9 |
| 15.2 | 13 |
| 15.5 | 68 |
| 15.9 | 86 |
| 16.3 | 12 |
| 17.3 | 83 |
| 17.5 | 43 |
| 18.3 | 38 |
| 18.8 | 37 |
| 19.3 | 71 |
| 19.8 | 56 |
| 20.1 | 31 |
| 20.3 | 22 |
| 20.8 | 100 |
| 21.7 | 29 |
| 22.0 | 54 |
| 22.2 | 36 |
| 22.8 | 61 |
| 23.2 | 19 |
| 23.5 | 22 |
| 23.7 | 17 |
| 24.6 | 56 |
| 25.9 | 16 |
| 26.5 | 25 |
| 26.9 | 28 |
| 27.4 | 20 |
| 28.4 | 23 |
| 28.8 | 14 |
| 29.7 | 28 |
| 30.5 | 15 |
| 31.3 | 13 |
| 32.0 | 15 |
| 32.3 | 37 |
| 33.1 | 14 |
| 35.4 | 12 |
| 36.2 | 16 |
| 37.3 | 15 |
| 38.9 | 14 |

### Formula I Mono-Malonate Tetrahydrofuran Solvate

Formula I mono-malonate tetrahydrofuran solvate was prepared by dissolving about 10 gr of amorphous Formula I freebase in 30 mL of tetrahydrofuran at ambient temperature. About 5 mL of water was charged, resulting in homogeneous solution upon agitation. About one molar equivalent of malonic acid was added as a solution of malonic acid in tetrahydrofuran. The solution was seeded with Formula I mono-malonate Form I. A slurry was obtained. About 45 mL of water were charged into the reactor and the contents were agitated for about a day. The slurry was sampled, and centrifuge filtered to obtain Formula I mono-malonate tetrahydrofuran solvate.

The bulk slurry was then filtered under vacuum and the filter cake was washed with a mixture of tetrahydrofuran and water. The wet solids were dried in the vacuum oven at about 40 °C for about 7 days to obtain Formula I mono-malonate tetrahydrofuran solvate.

XRPD analysis was conducted. FIG. 24 shows and XRPD pattern of the Formula I mono-malonate tetrahydrofuran solvate compound. XRPD peaks were identified and are included in Table 12 below.

TABLE 12

| Pos. [°2Th.] | Rel. Int. [ % ] |
|---|---|
| 5.5 | 1 |
| 7.5 | 6 |
| 7.9 | 37 |
| 12.4 | 1 |
| 13.7 | 2 |
| 13.9 | 2 |
| 15.7 | 100 |
| 16.0 | 6 |
| 16.2 | 3 |
| 17.4 | 3 |
| 17.6 | 4 |
| 19.0 | 4 |
| 19.4 | 8 |
| 20.0 | 8 |
| 20.5 | 4 |
| 21.1 | 74 |
| 21.7 | 4 |
| 22.2 | 8 |
| 22.7 | 7 |
| 23.7 | 4 |
| 24.0 | 15 |
| 24.8 | 53 |
| 26.3 | 3 |
| 26.8 | 2 |
| 27.1 | 4 |
| 28.7 | 27 |
| 30.0 | 10 |
| 30.7 | 4 |
| 31.9 | 3 |
| 32.6 | 20 |
| 33.6 | 4 |
| 34.5 | 3 |
| 35.5 | 2 |
| 36.6 | 6 |
| 37.6 | 3 |
| 39.2 | 4 |

### Formula I Xinafoate Form I

Formula I xinafoate Form I was prepared by adding 1 mL of methanol to about 80 mg of Formula I amorphous freebase. About one equivalent of 1-hydroxy-2-naphtoic acid was added. The mixture was stirred at ambient for about a day. A slurry was obtained. Formula I xinafoate Form I was obtained by isolating the solids by centrifugation.

XRPD analysis was conducted. FIG. 25 shows and XRPD pattern of the Formula I xinafoate Form I compound. XRPD peaks were identified and are included in Table 13 below.

TABLE 13

| Pos. [°2Th.] | Rel. Int. [ % ] |
|---|---|
| 4.3 | 59 |
| 6.7 | 28 |
| 8.9 | 48 |
| 12.4 | 28 |
| 12.7 | 16 |
| 13.2 | 33 |
| 15.5 | 100 |
| 15.8 | 33 |
| 16.8 | 17 |
| 17.9 | 29 |
| 18.4 | 16 |
| 19.8 | 12 |
| 20.9 | 20 |
| 22.8 | 14 |
| 24.1 | 11 |
| 24.8 | 18 |
| 25.7 | 9 |
| 26.5 | 9 |
| 27.8 | 7 |
| 28.5 | 8 |

### Formula I Xinafoate Form II

Formula I xinafoate Form I was dried in the vacuum oven at 50 °C for about 3 days to obtain Formula I xinafoate Form II.

XRPD analysis was conducted. FIG. 26 shows and XRPD pattern of the Formula I xinafoate Form II compound. XRPD peaks were identified and are included in Table 14 below.

TABLE 14

| Pos. [°2Th.] | Rel. Int. [ % ] |
|---|---|
| 4.6 | 23 |
| 6.6 | 24 |
| 9.1 | 33 |
| 9.4 | 79 |
| 12.3 | 15 |
| 12.8 | 32 |
| 13.2 | 56 |
| 13.5 | 81 |
| 15.3 | 97 |
| 15.5 | 52 |
| 15.9 | 24 |
| 16.6 | 17 |
| 16.9 | 39 |
| 18.3 | 44 |
| 18.9 | 100 |
| 19.8 | 13 |
| 21.2 | 44 |
| 21.8 | 21 |
| 22.8 | 10 |
| 23.4 | 6 |
| 23.8 | 7 |
| 24.7 | 9 |
| 25.8 | 15 |
| 26.5 | 22 |
| 27.2 | 14 |
| 32.2 | 4 |
| 33.7 | 5 |
| 38.7 | 4 |

DSC analysis was performed. FIG. 27 shows a DSC thermogram of the compound of Formula I xinafoate Form II. The DSC analysis revealed an endothermic transition with onset at about 45 °C and a second endothermic transition with an onset at about 124 °C.

TGA analysis was performed. FIG. 28 shows a TGA thermogram of the compound of Formula I xinafoate Form II. The TGA analysis indicates that the phase loses about 2.1% before degrading.

### Formula I Oxalate

Formula I oxalate was prepared by adding about 3.5 mL of acetonitrile to about 360 mg of Formula I amorphous freebase. About one molar equivalent of oxalic acid was added and the mixture was stirred at ambient for about a day. A slurry was obtained. About 2 mL of methyl-t-butyl ether was added. The slurry was stirred for a day and the solids were isolated by filtration. The wet solids were dried in the vacuum oven at about 40 °C for about 4 hours.

XRPD analysis was conducted. FIG. 29 shows and XRPD pattern of the Formula I oxalate. XRPD peaks were identified and are included in Table 15 below.

TABLE 15

| Pos. [°2Th.] | Rel. Int. [ % ] |
|---|---|
| 4.4 | 63 |
| 6.5 | 8 |
| 6.8 | 9 |
| 8.0 | 31 |
| 8.4 | 27 |
| 8.9 | 20 |
| 10.2 | 18 |
| 12.8 | 18 |
| 14.8 | 3 |
| 16.5 | 100 |
| 17.4 | 36 |
| 17.9 | 11 |
| 20.6 | 9 |
| 21.2 | 7 |
| 21.8 | 10 |
| 24.0 | 6 |
| 25.0 | 17 |
| 25.6 | 9 |
| 27.8 | 9 |
| 35.2 | 2 |

DSC analysis was performed. FIG. 30 shows a DSC thermogram of the compound of Formula I oxalate. The DSC analysis revealed an endothermic transition with onset at about 155 °C.

TGA analysis was performed. FIG. 31 shows a TGA thermogram of the compound of Formula I oxalate. The TGA analysis indicates two weight loss events about 4.6% degrading and 3.7% before degrading.

### Formula I Fumarate Form I

Formula I fumarate Form I was prepared by dissolving about 50 mg of Formula I amorphous freebase in 0.5 mL of methanol. About 4 molar equivalents of fumaric acid was added. The mixture was stirred at about 20 °C for about 6 days. The solid were isolated by centrifugation and dried in the vacuum oven at 50 °C for about a day.

XRPD analysis was conducted. FIG. 32 shows and XRPD pattern of the Formula I fumarate Form I compound. XRPD peaks were identified and are included in Table 16 below.

TABLE 16

| Pos. [°2Th.] | Rel. Int. [ % ] |
|---|---|
| 2.8 | 5 |
| 5.2 | 8 |
| 5.7 | 78 |
| 8.6 | 4 |
| 10.0 | 5 |
| 11.4 | 27 |
| 13.0 | 21 |
| 13.9 | 11 |
| 14.4 | 100 |
| 15.3 | 20 |
| 16.5 | 11 |
| 17.0 | 11 |
| 18.0 | 10 |
| 18.4 | 15 |
| 19.0 | 9 |
| 19.9 | 12 |
| 20.9 | 13 |
| 21.4 | 21 |
| 21.9 | 7 |
| 22.8 | 19 |
| 24.6 | 14 |
| 25.5 | 16 |
| 26.2 | 27 |
| 27.2 | 10 |
| 28.8 | 27 |
| 29.4 | 10 |
| 37.8 | 3 |

DSC analysis was performed. FIG. 33 shows a DSC thermogram of the compound of Formula I fumarate Form I. The DSC analysis revealed an endothermic transition with onset at about 31 °C and a second endothermic transition with an onset at about 145 °C.

TGA analysis was performed. FIG. 34 shows a TGA thermogram of the compound of Formula I fumarate Form I. The TGA analysis indicates two weight loss events about 1.1% before the second endothermic transition.

### Formula I Fumarate Form II

Formula I fumarate Form II was prepared by stirring about 100 mg of Formula I fumarate Form I in about 0.8 mL of ethyl acetate and stirring for about 3 days at about 22 °C. The solids were isolated by centrifugation and air-dried.

XRPD analysis was conducted. FIG. 35 shows and XRPD pattern of the Formula I fumarate Form II compound. XRPD peaks were identified and are included in Table 17 below.

TABLE 17

| Pos. [°2Th.] | Rel. Int. [ % ] |
|---|---|
| 5.6 | 24 |
| 6.1 | 56 |
| 10.7 | 12 |
| 11.1 | 21 |
| 12.3 | 20 |
| 12.7 | 37 |
| 13.4 | 55 |
| 13.6 | 61 |
| 15.6 | 65 |
| 16.2 | 79 |
| 16.5 | 100 |
| 18.4 | 47 |
| 19.0 | 20 |
| 20.0 | 35 |
| 20.9 | 25 |
| 21.4 | 35 |
| 22.5 | 29 |
| 24.7 | 88 |
| 25.2 | 65 |
| 26.3 | 52 |
| 27.5 | 31 |
| 28.3 | 31 |
| 30.0 | 18 |
| 30.7 | 13 |
| 37.0 | 21 |

DSC analysis was performed. FIG. 36 shows a DSC thermogram of the compound of Formula I fumarate Form II. The DSC analysis revealed an endothermic transition with an onset at about 25 °C and a second endothermic transition, which is likely a melting event, with an onset at about 162 °C. The two endothermic transitions are likely followed be a degradation event.

TGA analysis was performed. FIG. 37 shows a TGA thermogram of the compound of Formula I fumarate Form II. The TGA analysis indicates two weight loss events of about 1.2% before the second endothermic transition.

### Formula I Fumarate Form III

Formula I fumarate Form III was prepared by dissolving about 50 mg of Formula I amorphous freebase in 0.5 mL of methanol. About 3 molar equivalents of fumaric acid were added. The mixture was stirred at about 20 °C for about 13 days. The solids were isolated by centrifugation and dried in the vacuum oven at 50 °C for about 3 days.

XRPD analysis was conducted. FIG. 38 shows and XRPD pattern of the Formula I fumarate Form III compound. XRPD peaks were identified and are included in Table 18 below.

TABLE 18

| Pos. [°2Th.] | Rel. Int. [ % ] |
|---|---|
| 5.5 | 41 |
| 6.4 | 100 |
| 10.6 | 15 |
| 10.9 | 97 |
| 12.5 | 12 |
| 12.9 | 25 |
| 14.4 | 42 |
| 15.0 | 6 |
| 15.5 | 75 |
| 16.6 | 61 |
| 17.9 | 18 |
| 18.2 | 85 |
| 19.2 | 10 |
| 20.0 | 12 |
| 21.0 | 7 |
| 22.0 | 35 |
| 22.7 | 31 |
| 23.1 | 14 |
| 23.7 | 5 |
| 24.3 | 21 |
| 25.2 | 18 |
| 26.3 | 19 |
| 27.0 | 26 |
| 27.5 | 10 |
| 28.8 | 5 |
| 29.6 | 7 |
| 31.2 | 5 |
| 32.3 | 8 |
| 34.3 | 3 |
| 35.1 | 4 |
| 36.8 | 5 |
| 39.1 | 5 |

### Formula I Bis-Malonate

Formula I bis-malonate was prepared by dissolving about 500 mg of Formula I amorphous freebase in 5 mL of ethyl acetate. About 3 molar equivalents of malonic acid were added. The mixture was stirred for about a day. A slurry was obtained. The solids were isolated by filtration and dried in the vacuum oven at about 50 °C for about 3 days.

Formula I bis-malonate was also prepared by dissolving about 100 mg of Formula I amorphous freebase in about 0.8 mL of ethyl acetate, followed by adding 2, 3, 4, 5 or 10 molar equivalents of malonic acid. The mixtures were stirred at ambient for about 4 days. The wet cakes were isolated by centrifugation and dried in the vacuum oven at about 50 °C.

XRPD analysis was conducted. FIG. 39 shows and XRPD pattern of the Formula I bis-malonate compound. XRPD peaks were identified and are included in Table 19 below.

TABLE 19

| Pos. [°2Th.] | Rel. Int. [%] |
|---|---|
| 6.9 | 54 |
| 8.2 | 19 |
| 10.3 | 4 |
| 11.2 | 27 |
| 11.6 | 58 |
| 12.5 | 23 |
| 12.9 | 63 |
| 13.5 | 100 |
| 14.0 | 19 |
| 15.0 | 39 |
| 15.7 | 13 |
| 16.2 | 40 |
| 17.1 | 31 |
| 18.4 | 60 |
| 20.4 | 63 |
| 21.2 | 35 |
| 22.6 | 26 |
| 23.6 | 45 |
| 25.1 | 38 |
| 27.1 | 22 |
| 28.0 | 15 |
| 29.8 | 15 |
| 31.0 | 5 |
| 35.4 | 4 |

DSC analysis was performed. FIG. 40 shows a DSC thermogram of the compound of Formula I bis-malonate. The DSC analysis revealed an endothermic transition with onset at about 114 °C.

TGA analysis was performed. FIG. 41 shows a TGA thermogram of the compound of Formula I bis-malonate. The TGA analysis indicates a weight loss event of about 1.6% before degrading.

### Formula I L-Tartrate

Formula I L-tartrate was prepared by suspending about 40 mg of Formula I amorphous freebase in 0.4 mL of acetonitrile. Approximately one molar equivalent of L-tartaric acid was added, and the mixture was stirred at ambient temperature for about 11 days. A solution was obtained. The acetonitrile was removed by rotary evaporation and 0.4 mL of methanol was added. No slurry was obtained. The methanol was removed by rotary evaporation and 0.3 mL of ethyl acetate was added. After about five days a thick slurry was obtained. The solids were isolated by centrifugation and dried in a vacuum oven at about 50 °C for about a day.

XRPD analysis was conducted. FIG. 42 shows and XRPD pattern of the Formula I L-tartrate compound. XRPD peaks were identified and are included in Table 20 below.

TABLE 20

| Pos. [°2Th.] | Rel. Int. [%] |
|---|---|
| 4.7 | 26 |
| 6.4 | 100 |
| 9.6 | 14 |
| 13.2 | 64 |
| 13.8 | 61 |
| 15.6 | 59 |
| 16.4 | 39 |
| 17.5 | 44 |
| 18.8 | 64 |
| 20.8 | 84 |
| 21.8 | 43 |
| 22.7 | 40 |
| 25.1 | 42 |
| 36.8 | 30 |

DSC analysis was performed. FIG. 43 shows a DSC thermogram of the compound of Formula I L-tartrate. The DSC analysis revealed a broad endothermic transition followed by a second endothermic transition with onset at about 156 °C.

TGA analysis was performed. FIG. 44 shows a TGA thermogram of the compound of Formula I L-tartrate. The TGA analysis indicates a weight loss event of about 1.6% before degrading.

### Formula I Mono-Malonate Dichloromethane Solvate

Formula I mono-malonate dichloromethane solvate was prepared by adding around 0.4 mL of dichloromethane to about 50 mg of GS-1427 mono-malonate Form II. The mixture was stirred at ambient for about 3 days and the slurry was filtered with centrifugation to obtain GS-1427 mono-malonate dichloromethane solvate.

XRPD analysis was conducted. FIG. 52 shows and XRPD pattern of the Formula I mono-malonate dichloromethane solvate compound. XRPD peaks were identified and are included in Table 21 below.

TABLE 21

| Pos. [°2Th.] | Rel. Int. [%] |
|---|---|
| 7.5 | 99 |
| 7.8 | 45 |
| 11.5 | 23 |
| 12.2 | 24 |
| 13.3 | 12 |
| 13.8 | 64 |
| 15.7 | 70 |
| 16.0 | 65 |
| 17.5 | 47 |
| 19.1 | 44 |
| 19.5 | 47 |
| 20.2 | 42 |
| 21.0 | 82 |
| 22.3 | 45 |
| 23.2 | 38 |
| 24.8 | 100 |
| 27.2 | 10 |
| 28.7 | 20 |
| 29.9 | 15 |
| 32.6 | 18 |

### Formula I Mono-Malonate Methyl Ethyl Ketone Solvate

Formula I mono-malonate methyl ethyl ketone solvate was prepared by adding around 0.4 mL of methyl ethyl ketone to about 50 mg of GS-1427 mono-malonate Form II. The mixture was stirred at ambient for about 19 days and the slurry was filtered with centrifugation to obtain GS-1427 mono-malonate methyl ethyl ketone solvate.

XRPD analysis was conducted. FIG. 52 shows and XRPD pattern of the Formula I mono-malonate methyl ethyl ketone solvate compound. XRPD peaks were identified and are included in Table 22 below.

TABLE 22

| Pos. [°2Th.] | Rel. Int. [%] |
|---|---|
| 5.4 | 5 |
| 7.4 | 100 |
| 7.9 | 22 |
| 8.4 | 2 |
| 11.4 | 12 |
| 12.1 | 15 |
| 13.0 | 7 |
| 13.6 | 38 |
| 14.8 | 5 |
| 15.5 | 16 |
| 15.7 | 41 |
| 16.0 | 62 |
| 17.4 | 49 |
| 17.6 | 24 |
| 18.3 | 18 |
| 18.9 | 36 |
| 19.4 | 52 |
| 19.9 | 29 |
| 20.2 | 25 |
| 20.5 | 12 |
| 21.0 | 66 |
| 21.8 | 23 |
| 22.1 | 38 |
| 22.3 | 23 |
| 22.6 | 20 |
| 22.9 | 42 |
| 23.3 | 12 |
| 23.7 | 20 |
| 24.2 | 14 |
| 24.7 | 46 |
| 25.3 | 11 |
| 26.0 | 11 |
| 26.3 | 7 |
| 26.7 | 14 |
| 27.1 | 16 |
| 27.3 | 8 |
| 27.5 | 10 |
| 28.3 | 8 |
| 28.6 | 14 |
| 30.0 | 16 |
| 30.7 | 13 |
| 32.5 | 22 |
| 33.5 | 9 |
| 35.5 | 8 |
| 36.2 | 9 |
| 37.4 | 9 |
| 39.1 | 12 |

### Compounds of Formula II

### Formula II Freebase Form I

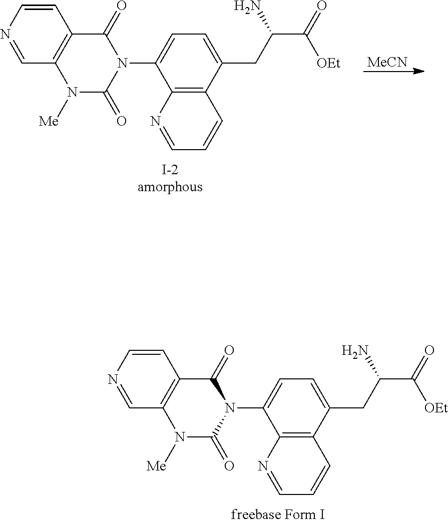

Amorphous I-2 was stirred in about 5 volumes of acetonitrile at about 20 °C for about 3 hours. The slurry was filtered under vacuum to afford Formula II freebase Form I.

XRPD analysis was conducted. FIG. 43 shows and XRPD pattern of the Formula II freebase Form I. XRPD peaks were identified and are included in Table 23 below.

TABLE 23

| Pos. [°2Th.] | Rel. Int. [%] |
|---|---|
| 6.8 | 25 |
| 7.2 | 100 |
| 8.0 | 28 |
| 9.9 | 39 |
| 12.5 | 16 |
| 13.6 | 7 |
| 14.7 | 9 |
| 15.3 | 10 |
| 15.9 | 1 |
| 16.4 | 9 |
| 17.6 | 16 |
| 18.0 | 11 |
| 18.4 | 3 |
| 18.8 | 3 |
| 19.5 | 2 |
| 19.8 | 13 |
| 20.3 | 2 |
| 20.6 | 11 |
| 21.6 | 7 |
| 21.9 | 9 |
| 22.6 | 9 |
| 23.7 | 8 |
| 24.2 | 3 |
| 25.5 | 30 |
| 25.9 | 20 |
| 26.2 | 19 |
| 27.2 | 4 |
| 27.5 | 4 |
| 28.1 | 9 |
| 28.8 | 2 |
| 30.8 | 4 |
| 32.1 | 3 |
| 32.6 | 2 |
| 33.5 | 2 |
| 34.4 | 2 |
| 34.7 | 2 |
| 36.5 | 4 |
| 38.0 | 4 |

### Formula I Freebase Form II

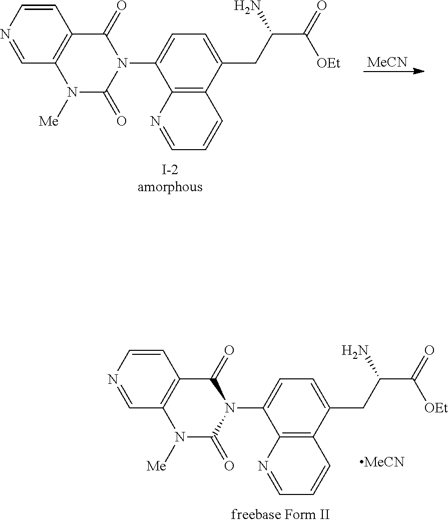

Amorphous I-2 was stirred in about 5 to 10 volumes of acetonitrile at about 20 °C. A slurry formed, and the mixture was heated to about 50 °C and then cooled to about 20 °C. The slurry was heated again to about 50 °C and held at constant temperature for around 12 hours and then cooled to about 20 °C over about 12 hours and aged at about 20 °C for about 2 days. The slurry was filtered under vacuum and washed with about 2 volumes of isopropyl acetate. The filter cake was dried at about 50 °C under vacuum for about 1 day to afford Formula II freebase Form II as a partial acetonitrile solvate.

XRPD analysis was conducted. FIG. 44 shows and XRPD pattern of the Formula II freebase Form II. XRPD peaks were identified and are included in Table 24 below.

TABLE 24

| Pos. [°2Th.] | Rel. Int. [%] |
|---|---|
| 8.6 | 35 |
| 9.8 | 1 |
| 12.2 | 5 |
| 12.6 | 7 |
| 14.0 | 61 |
| 14.8 | 80 |
| 16.4 | 11 |
| 16.7 | 23 |
| 17.2 | 13 |
| 17.8 | 26 |
| 18.4 | 20 |
| 19.7 | 45 |
| 20.4 | 20 |
| 20.9 | 39 |
| 21.4 | 37 |
| 22.0 | 6 |
| 22.3 | 28 |
| 23.1 | 2 |
| 23.8 | 8 |
| 24.2 | 100 |
| 24.5 | 34 |
| 25.2 | 9 |
| 25.9 | 4 |
| 26.5 | 11 |
| 27.6 | 32 |
| 28.2 | 8 |
| 29.7 | 30 |
| 31.2 | 4 |
| 31.9 | 5 |
| 32.2 | 4 |
| 32.8 | 4 |
| 33.3 | 8 |
| 33.5 | 10 |
| 34.1 | 5 |
| 34.9 | 1 |
| 36.0 | 3 |
| 36.3 | 4 |
| 37.1 | 1 |

DSC analysis was performed. FIG. 45 shows a DSC thermogram of the compound of Formula II freebase Form II. The DSC analysis revealed a broad endothermic transition with an onset at about 104 °C, followed by a second endothermic transition with onset at about 152 °C.

TGA analysis was performed. FIG. 46 shows a TGA thermogram of the compound of Formula II freebase Form II. The TGA analysis indicates a weight loss event of about 0.3% before the first endothermic transition, about 2.5% weight loss during the first endothermic transition and about 1.0% during the second endothermic transition.

### Formula II Freebase Form III

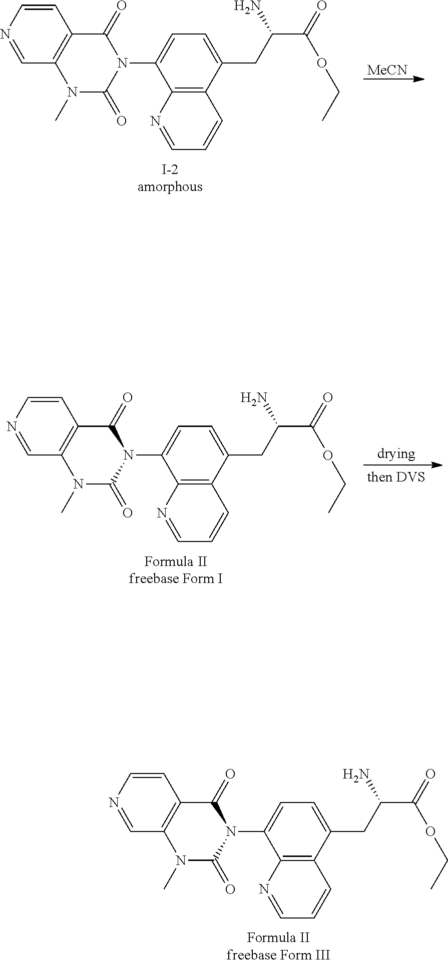

Amorphous I-2 was stirred in about 5 volumes of acetonitrile at about 20 °C for about 3 hours. The slurry was filtered under vacuum to afford Formula II freebase Form I.

The filter cake solids were dried in a vacuum oven at about 25 °C for about 3 days. A sample of the dried solids was exposed to relative humidities between 0 and 90%, using dynamic vapor sorption (DVS) analysis. The post-DVS analysis solids were analyzed with XRPD, to obtain Formula II freebase Form III.

XRPD analysis was conducted. FIG. 47 shows and XRPD pattern of the Formula II freebase Form III. XRPD peaks were identified and are included in Table 25 below.

TABLE 25

| Pos. [°2Th.] | Rel. Int. [%] |
|---|---|
| 6.7 | 5 |
| 7.3 | 100 |
| 8.1 | 16 |
| 10.0 | 14 |
| 12.5 | 8 |
| 15.2 | 4 |
| 16.2 | 25 |
| 17.9 | 22 |
| 20.0 | 6 |
| 22.4 | 5 |
| 23.9 | 4 |
| 25.7 | 16 |
| 25.9 | 17 |

### Formula II Freebase Form IV

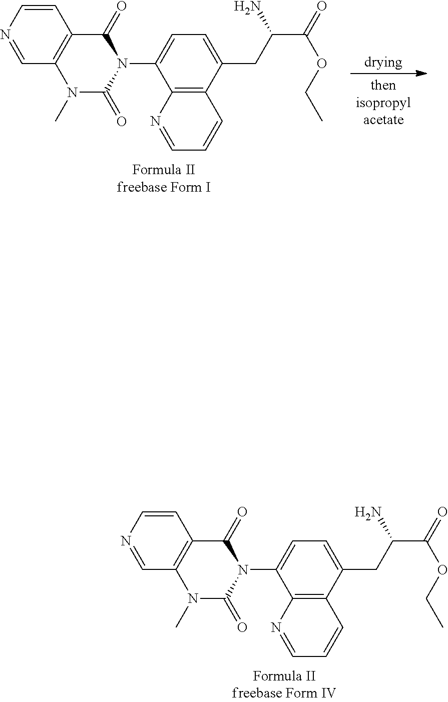

Formula II freebase Form I was dried in a vacuum oven at 25 °C for about 3 days. Solids were then stirred in isopropyl acetate at about 20 °C until a slurry formed. The slurry was stirred at about 20 °C for about 2 weeks. The slurry was sampled with XRPD to obtain Formula II freebase Form IV.

Formula II freebase Form IV can also be prepared by drying Formula II freebase Form II at about 125 °C for about 20 minutes.

Formula II freebase Form IV can also be prepared by charging Formula II freebase Form VI solids in isopropyl acetate at about 20 °C until slurry is formed. The slurry was stirred at about 20 °C for about 6 days. The slurry was sampled with XRPD to obtain Formula II freebase Form IV.

Formula II freebase Form IV can also be prepared by charging Formula II freebase Form VI solids in chlorobenzene at about 20 °C until slurry is formed. The slurry was stirred at about 20 °C for about 4 days. The slurry was sampled with XRPD to obtain Formula II freebase Form IV.

XRPD analysis was conducted. FIG. 48 shows and XRPD pattern of the Formula II freebase Form IV. XRPD peaks were identified and are included in Table 26 below.

TABLE 26

| Pos. [°2Th.] | Rel. Int. [%] |
|---|---|
| 4.7 | 41 |
| 10.2 | 3 |
| 11.0 | 61 |
| 11.3 | 26 |
| 11.5 | 32 |
| 12.5 | 100 |
| 13.9 | 8 |
| 14.1 | 6 |
| 15.9 | 8 |
| 16.4 | 66 |
| 17.8 | 8 |
| 19.5 | 7 |
| 20.1 | 7 |
| 20.5 | 3 |
| 21.2 | 7 |
| 23.2 | 7 |
| 25.2 | 6 |
| 26.5 | 4 |
| 27.8 | 7 |
| 30.0 | 2 |
| 32.0 | 2 |

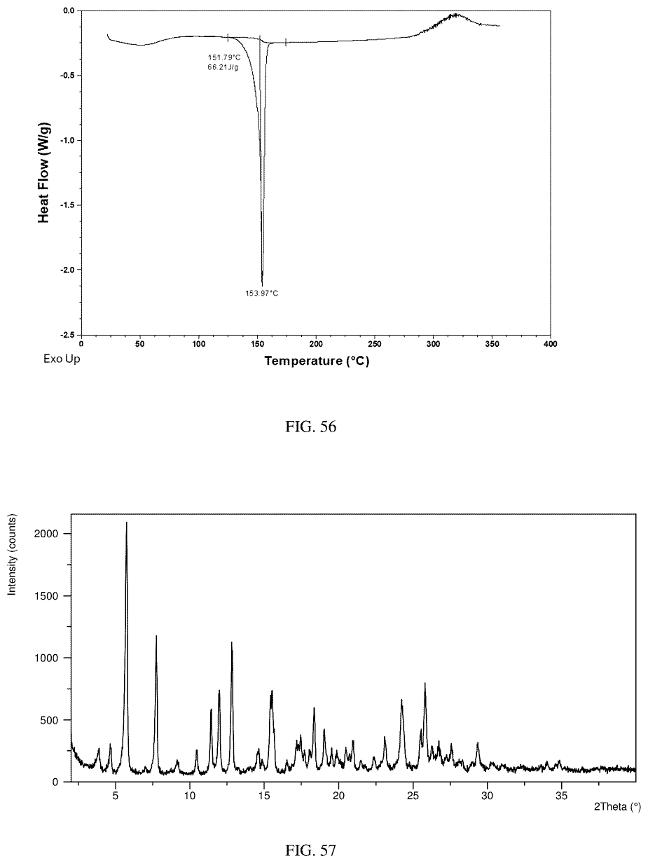

DSC analysis was performed. FIG. 56 shows a DSC thermogram of the compound of Formula II freebase Form IV. The DSC analysis revealed an endothermic transition with an onset at about 151.8 °C.

### Formula H Freebase Form V

Amorphous I-2, concentrated from the liquors of Formula II freebase Form I, was mixed in 10 volumes of ethanol and 4.0 equiv methanesulfonic acid and heated at about 70 °C for about 20 hours. Cooled to about 20 °C and diluted with 10 volumes of dichloromethane and 5 volumes of aqueous 10% ammonium hydroxide solution. Separated the layers and concentrated the organic layer to dryness. Charged 5 volumes acetonitrile and reconcentrated to dryness. Charged 5 volumes acetonitrile and solids of Form I that were dried at about 25 °C for about 3 days. Stirred at about 20 °C for about 16 hours but negligible seed bed formed. Concentrated to dryness and charged 5 volumes acetonitrile to get a thin slurry. Concentrated to dryness and diluted with 10 volumes dichloromethane and 3 volumes of aqueous 10% ammonium hydroxide solution. Separated the layers and washed the organic layer with 3 volumes of water. Concentrated the organic layer to dryness and charged 5 volumes acetonitrile to form a slurry. The slurry was next stirred at about 20 °C for about 20 hours. The slurry was heated to about 50 °C and then cooled to about 20 °C three times over about 2 days. The slurry was filtered under vacuum and washed with about 2 volumes of isopropyl acetate. The filter cake was dried at about 50 °C under vacuum for about 1 day to afford Formula II freebase Form V.

XRPD analysis was conducted. FIG. 49 shows an XRPD pattern of the Formula II freebase Form V. XRPD peaks were identified and are included in Table 27 below.

TABLE 27

| Pos. [°2Th.] | Rel. Int. [%] |
|---|---|
| 8.6 | 50 |
| 9.9 | 5 |
| 12.4 | 14 |
| 14.3 | 40 |
| 15.0 | 52 |
| 16.7 | 38 |
| 17.2 | 23 |
| 17.9 | 39 |
| 18.5 | 23 |
| 19.8 | 64 |
| 20.7 | 20 |
| 21.3 | 34 |
| 21.6 | 63 |
| 22.6 | 23 |
| 23.8 | 12 |
| 24.5 | 100 |
| 25.2 | 15 |
| 25.9 | 7 |
| 26.8 | 15 |
| 27.9 | 30 |
| 28.7 | 10 |
| 30.2 | 22 |
| 31.8 | 8 |
| 32.4 | 8 |
| 33.3 | 18 |
| 34.1 | 11 |
| 37.1 | 6 |

### Formula II Freebase Form VI

Formula II freebase Form II was dried in a vacuum oven at 70 °C for about 11 days to obtain Formula II freebase Form VI.

XRPD analysis was conducted. FIG. 57. shows an XRPD pattern of the Formula II freebase Form VI. XRPD peaks were identified and are included in Table 28 below.

TABLE 28

| Pos. [°2Th.] | Rel. Int. [%] |
|---|---|
| 3.9 | 8 |
| 4.7 | 9 |
| 5.8 | 100 |
| 7.0 | 2 |
| 7.8 | 51 |
| 9.2 | 5 |
| 10.5 | 9 |
| 11.4 | 27 |
| 12.0 | 34 |
| 12.8 | 51 |
| 14.6 | 9 |
| 14.9 | 5 |
| 15.4 | 31 |
| 15.5 | 34 |
| 16.5 | 4 |
| 17.1 | 11 |
| 17.5 | 15 |
| 18.1 | 9 |
| 18.4 | 27 |
| 19.0 | 18 |
| 19.5 | 10 |
| 19.9 | 8 |
| 20.5 | 10 |
| 21.0 | 13 |
| 21.5 | 5 |
| 22.4 | 6 |
| 23.1 | 14 |
| 24.2 | 28 |
| 25.5 | 17 |
| 25.8 | 36 |
| 26.3 | 11 |
| 26.7 | 10 |
| 27.6 | 11 |
| 28.3 | 4 |
| 29.3 | 11 |
| 30.3 | 3 |
| 31.0 | 3 |
| 32.3 | 2 |
| 34.0 | 3 |
| 34.8 | 4 |

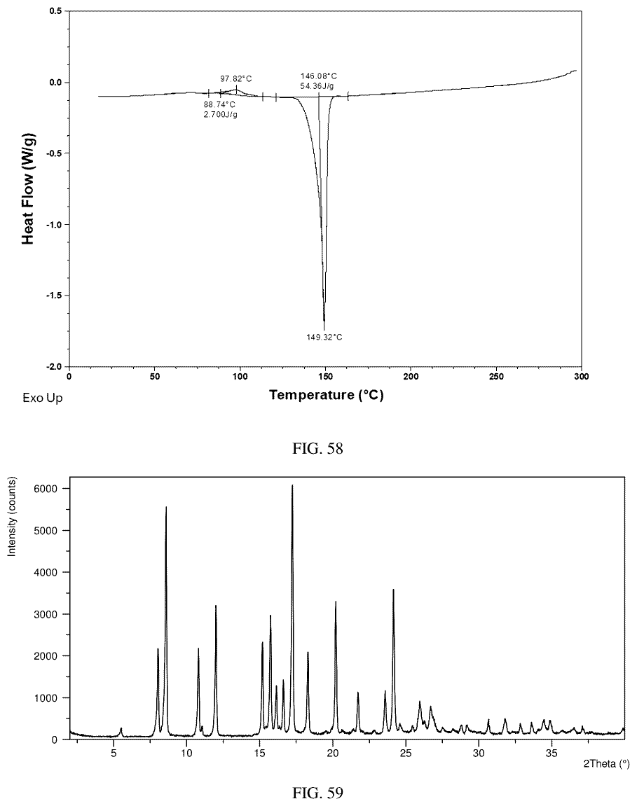

DSC analysis was performed. FIG. 58 shows a DSC thermogram of the compound of Formula II freebase Form VI. The DSC analysis revealed an endothermic transition with an onset at about 88.7 °C and a second endothermic transition with an onset at about 146.1 °C.

### Formula II Freebase Form VII

Formula II freebase Form VI solids were stirred in anisole at about 20 °C until a slurry formed. The slurry was stirred at about 20 °C for about 4 days. The slurry was sampled with XRPD to obtain Formula II freebase Form VII.

XRPD analysis was conducted. FIG. 59. shows an XRPD pattern of the Formula II freebase Form VII. XRPD peaks were identified and are included in Table 29 below.

TABLE 29

| Pos. [°2Th.] | Rel. Int. [%] |
|---|---|
| 5.5 | 3 |
| 8.0 | 34 |
| 8.6 | 87 |
| 10.8 | 34 |
| 11.1 | 4 |
| 12.0 | 51 |
| 15.2 | 37 |
| 15.7 | 47 |
| 16.1 | 19 |
| 16.6 | 22 |
| 17.2 | 100 |
| 18.3 | 32 |
| 19.5 | 2 |
| 20.2 | 52 |
| 21.7 | 17 |
| 22.9 | 2 |
| 23.6 | 17 |
| 24.2 | 57 |
| 24.6 | 4 |
| 25.5 | 4 |
| 26.0 | 13 |
| 26.3 | 5 |
| 26.7 | 11 |
| 27.5 | 3 |
| 28.3 | 2 |
| 28.8 | 4 |
| 29.2 | 4 |
| 30.6 | 5 |
| 31.8 | 6 |
| 32.8 | 4 |
| 33.6 | 5 |
| 34.4 | 6 |
| 34.9 | 6 |
| 35.7 | 1 |
| 36.5 | 2 |
| 37.1 | 3 |

### Formula II Freebase Form VIII

Formula II freebase Form VI solids were stirred in ethanol at about 20 °C until a slurry formed. The slurry was stirred at about 20 °C for about 6 days. The slurry was sampled with XRPD to obtain Formula II freebase Form VIII.

XRPD analysis was conducted. FIG. 60. shows an XRPD pattern of the Formula II freebase Form VIII. XRPD peaks were identified and are included in Table 30 below.

TABLE 30

| Pos. [°2Th.] | Rel. Int. [%] |
|---|---|
| 7.0 | 98 |
| 7.4 | 100 |
| 9.1 | 10 |
| 9.9 | 23 |
| 10.4 | 16 |
| 13.1 | 3 |
| 14.8 | 16 |
| 15.2 | 19 |
| 16.1 | 20 |
| 16.5 | 26 |
| 17.7 | 10 |
| 18.3 | 9 |
| 19.7 | 5 |
| 20.8 | 10 |
| 21.2 | 11 |
| 22.5 | 13 |
| 24.4 | 11 |
| 25.7 | 18 |
| 26.8 | 13 |
| 35.0 | 2 |

### Formula II Freebase Form IX

Formula II freebase Form VI solids were charged in THF, MeTHF, or MEK at about 20 °C until a slurry formed. The slurry was stirred at about 20 °C for about 6 days. The slurry was sampled with XRPD to obtain Formula II freebase Form IX.

XRPD analysis was conducted. FIG. 61. shows an XRPD pattern of the Formula II freebase Form IX. XRPD peaks were identified and are included in Table 31 below.

TABEL 31

| Pos. [°2Th.] | Rel. Int. [%] |
|---|---|
| 6.6 | 18 |
| 7.2 | 100 |
| 7.9 | 28 |
| 9.8 | 33 |
| 12.3 | 13 |
| 13.4 | 22 |
| 14.6 | 21 |
| 14.8 | 16 |
| 15.2 | 19 |
| 16.3 | 23 |
| 17.5 | 32 |
| 17.9 | 36 |
| 18.6 | 4 |
| 19.7 | 22 |
| 20.0 | 3 |
| 20.5 | 21 |
| 21.7 | 5 |
| 21.9 | 14 |
| 22.4 | 9 |
| 22.7 | 4 |
| 23.6 | 17 |
| 24.0 | 6 |
| 25.6 | 46 |
| 25.7 | 42 |
| 26.1 | 74 |
| 26.9 | 5 |
| 27.3 | 16 |
| 27.9 | 26 |
| 28.9 | 3 |
| 30.1 | 4 |
| 30.7 | 8 |
| 32.1 | 4 |
| 32.7 | 3 |
| 33.6 | 3 |
| 34.4 | 6 |
| 36.3 | 6 |
| 37.3 | 3 |
| 38.0 | 8 |

### Formula I Freebase Form X

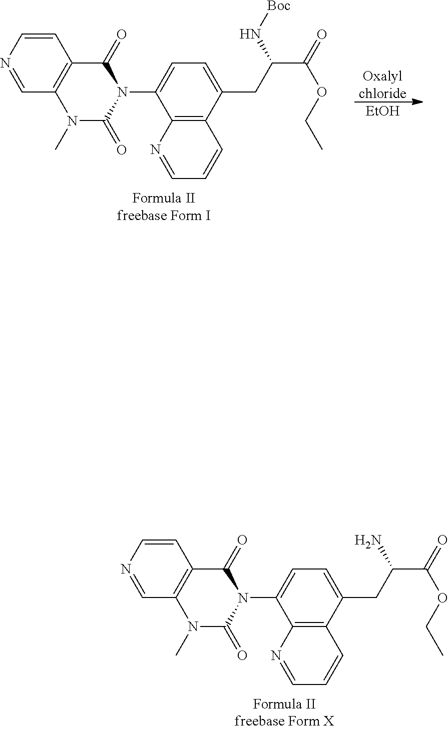

Formula II freebase Form I was mixed in 10 volumes of ethanol at about 20 °C and 3 equiv of oxalyl chloride were charged dropwise. After about 4 days, the contents were concentrated to around 4 volumes, cooled to about 20 °C, diluted with dichloromethane and aqueous ammonium hydroxide solution. The layers were separated, and the organic layer was concentrated to about 4 volumes. The organic layer was charged with 8.5 volumes of acetonitrile and reconcentrated to 4 volumes. Charged again with 8.5 volumes of acetonitrile and reconcentrated to 4 volumes. Finally, the resulting solution was charged with 6 volumes of acetonitrile. An aliquot was removed from the solution and crystallized overnight at ambient temperature. Single-crystal structure determination was performed, confirming a stoichiometry of Formula II freebase Form X.

A diffractogram, was indexed and the unit cell dimensions were calculated. Results are reported in Table 32 below. Data collection was performed on a Rigaku SuperNova diffractometer, equipped with a copper anode microfocus sealed X-ray tube (Cu Ka 1=1.54184 Å) and a Dectris Pilatus3 R 200K hybrid pixel array detector. Cell constants and an orientation matrix for data collection were obtained from least-squares refinement using the setting angles of 6348 reflections in the range 3.9410°<q<75.5860°. The space group was determined by the program CRYSALISPRO [CrysAlisPro 1.171.41.93a (Rigaku Oxford Diffraction, 2020)] to be P21212 (international tables no. 18).

The data were collected to a maximum diffraction angle (2θ) of 151.986° at a temperature of 150K.

TABLE 32

| Space group | P2₁2₁2 | Volume (Å³) | 2155.86(10) |
|---|---|---|---|
| Unit cell parameters | | | |
| a (Å) | 6.6364(2) | α (°) | = 90 |
| b (Å) | 26.2974(6) | β (°) | = 90 |
| c (Å) | 12.3531(3) | γ (°) | = 90 |

XRPD was generated from a single crystal structure using Mercury, FIG. 62. shows an XRPD pattern of the Formula II freebase Form X. XRPD peaks were identified and are included in Table 33 below.

TABLE 33

| Pos. [°2Th.] | Rel. Int. [%] |
|---|---|
| 6.7 | 24 |
| 7.2 | 100 |
| 7.9 | 41 |
| 9.8 | 33 |
| 12.4 | 15 |
| 13.5 | 6 |
| 13.8 | 5 |
| 14.7 | 8 |
| 14.9 | 23 |
| 15.3 | 13 |
| 15.5 | 13 |
| 15.8 | 3 |
| 16.6 | 18 |
| 16.7 | 9 |
| 17.6 | 21 |
| 18.2 | 27 |
| 19.0 | 5 |
| 19.6 | 3 |
| 19.9 | 21 |
| 20.3 | 3 |
| 20.8 | 20 |
| 21.6 | 5 |
| 21.8 | 7 |
| 22.1 | 1 |
| 22.7 | 13 |
| 23.9 | 17 |
| 24.3 | 4 |
| 25.4 | 3 |
| 25.7 | 38 |
| 26.0 | 34 |
| 26.3 | 3 |
| 26.8 | 67 |
| 27.3 | 4 |
| 27.4 | 4 |
| 27.8 | 6 |
| 28.0 | 16 |
| 28.6 | 26 |
| 28.9 | 4 |
| 29.7 | 3 |
| 30.3 | 1 |
| 30.8 | 5 |
| 30.9 | 3 |
| 31.3 | 5 |
| 32.0 | 3 |
| 32.7 | 5 |
| 33.6 | 3 |
| 33.9 | 1 |
| 34.3 | 1 |
| 34.9 | 9 |
| 35.5 | 1 |
| 36.2 | 3 |
| 36.3 | 3 |
| 36.6 | 5 |
| 37.5 | 3 |
| 37.8 | 2 |
| 38.1 | 6 |
| 38.3 | 1 |
| 39.0 | 1 |
| 40.0 | 1 |
| 40.4 | 1 |
| 40.7 | 2 |
| 41.3 | 1 |
| 42.4 | 2 |
| 42.8 | 5 |
| 43.0 | 1 |
| 43.6 | 2 |
| 43.8 | 2 |
| 44.1 | 2 |
| 44.5 | 1 |
| 45.1 | 1 |
| 45.3 | 2 |
| 45.7 | 3 |
| 45.9 | 5 |
| 46.5 | 1 |
| 46.7 | 2 |
| 47.0 | 2 |
| 47.4 | 2 |
| 47.7 | 1 |
| 48.2 | 1 |
| 49.0 | 0 |
| 49.4 | 1 |

### Formula III Compound

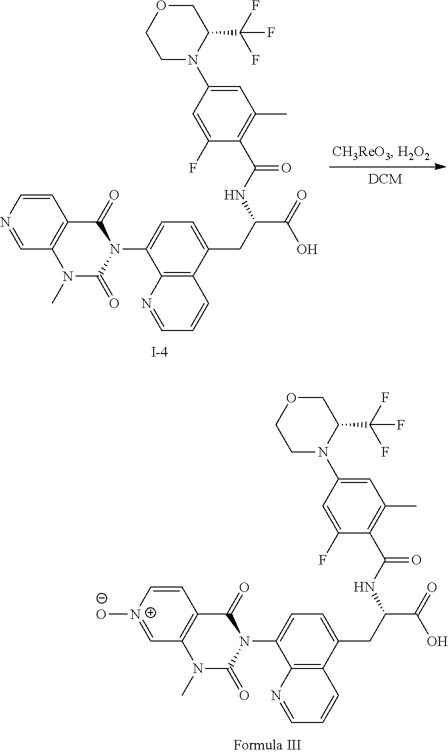

Synthesis of (2S)-2-[[2-fluoro-6-methyl-4-[(3R)-3-(trifluoromethyl)morpholin-4-yl]benzoyl]amino]-3-[8-[(3S)-1-methyl-7-oxido-2,4-dioxo-pyrido[3,4-d]pyrimidin-7-ium-3-yl]-5-quinolyl]propanoic acid (Formula III): To a vial containing (2S)-2-(2-fluoro-6-methyl-4-((R)-3-(trifluoromethyl)morpholino)benzamido)-3-(8-(1-methyl-2,4-dioxo-1,4-dihydropyrido[3,4-d]pyrimidin-3(2H)-yl)quinolin-5-yl)propanoic acid, I-4 (56.0 mg, 0.082 mmol) and DCM (2.0 mL) was added methyltrioxorhenium (3.0 mg, 0.0085 mmol) and the mixture was cooled in an ice bath. To the cooled mixture was slowly added a 50 w % aqueous solution of H₂O₂ (0.411 mmol, 42 μL) and the mixture was allowed to warm to room temperature and stirred for about 72 hrs. The solvent was removed in vacuo and the crude product was dissolved in DMSO and purified by preparatory HPLC to afford the title compounds, Formula III ES/MS (m/z) 697.2 (M+H)+. 1H NMR (400 MHz, DMSO-d6) δ 8.88-8.83 (m, 2H), 8.67 (d, J=4.5 Hz, 2H), 8.13 (d, J=6.6 Hz, 1H), 7.91 (d, J=6.7 Hz, 1H), 7.74 (s, 1H), 7.69-7.65 (m, 2H), 6.71-6.63 (m, 2H), 4.88-4.70 (m, 2H), 4.14 (d, J=12.6 Hz, 3H), 3.94 (dd, J=12.8, 1.9 Hz, 2H), 3.72 (d, J=11.3 Hz, 3H), 3.55 (d, J=8.6 Hz, 1H), 3.51 (s, 3H), 3.49-3.41 (m, 1H), 3.35 (d, J=12.0 Hz, 1H), 3.26 (d, J=17.8 Hz, 1H), 2.02 (s, 3H).

### Formula IV Compound

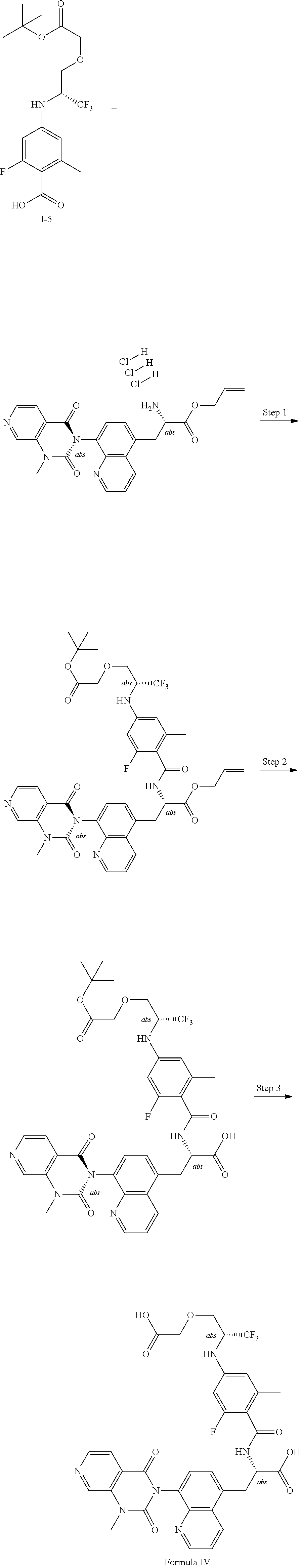

Step 1. Synthesis of allyl (2S)-2-[[4-[[(1R)-1-[(2-tert-butoxy-2-oxo-ethoxy)methyl]-2,2,2-trifluoro-ethyl]amino]-2-fluoro-6-methyl-benzoyl]amino]-3-[8-(1-methyl-2,4-dioxo-pyrido[3,4-d]pyrimidin-3-yl)-5-quinolyl]propanoate: To a solution of 4-[[(1R)-1-[(2-tert-butoxy-2-oxo-ethoxy)methyl]-2,2,2-trifluoro-ethyl]amino]-2-fluoro-6-methyl-benzoic acid (I-5): (130 mg, 0.32 mmol) and DIPEA (0.55 mL, 3.2 mmol) in DCM (5.0 mL) was added allyl (2S)-2-amino-3-[8-(1-methyl-2,4-dioxo-pyrido[3,4-d]pyrimidin-3-yl)-5-quinolyl]propanoate trihydrochloride (190 mg, 0.35 mmol) and HATU (130 mg, 0.55 mmol). The solution was stirred at ambient temp for about 1.5 hours at which point it was quenched with 10% aq. KHSO₄ and extracted 3× with DCM. The combined extracts were dried over Na₂SO₄, filtered, and evaporated to give crude residue. This was purified via flash chromatography (100% heptanes→100% [3:1 EtOAc:EtOH]) to afford title compound. ES/MS (m/z) 809.3 (M+H)+.

Step 2. Synthesis of (2S)-2-[[4-[[(1R)-1-[(2-tert-butoxy-2-oxo-ethoxy)methyl]-2,2,2-trifluoro-ethyl]amino]-2-fluoro-6-methyl-benzoyl]amino]-3-[8-(1-methyl-2,4-dioxo-pyrido[3,4-d]pyrimidin-3-yl)-5-quinolyl]propanoic acid: To a solution of allyl (2S)-2-[[4-[[(1R)-1-[(2-tert-butoxy-2-oxo-ethoxy)methyl]-2,2,2-trifluoro-ethyl]amino]-2-fluoro-6-methyl-benzoyl]amino]-3-[8-(1-methyl-2,4-dioxo-pyrido[3,4-d]pyrimidin-3-yl)-5-quinolyl]propanoate (260 mg, 0.30 mmol) in THF (2.9 mL) was added morpholine (0.13 mL, 1.5 mmol) and the solution stirred at ambient temp. Pd(PPh₃)₄ (18 mg, 0.02 mmol) was then added and the solution was stirred for about 45 minutes at which point the solution was evaporated. The residue was purified via flash chromatography (100% DCM 450% MeOH/DCM) to afford title compound. ES/MS (m/z) 769.3 (M+H)+.

Step 3. Synthesis of (2S)-2-[[4-[[(1R)-1-(carboxymethoxymethyl)-2,2,2-trifluoro-ethyl]amino]-2-fluoro-6-methyl-benzoyl]amino]-3-[8-(1-methyl-2,4-dioxo-pyrido[3,4-d]pyrimidin-3-yl)-5-quinolyl]propanoic acid (Formula IV): (2S)-2-[[4-[[(1R)-1-[(2-tert-butoxy-2-oxo-ethoxy)methyl]-2,2,2-trifluoro-ethyl]amino]-2-fluoro-6-methyl-benzoyl]amino]-3-[8-(1-methyl-2,4-dioxo-pyrido[3,4-d]pyrimidin-3-yl)-5-quinolyl]propanoic acid (190 mg, 0.25 mmol) was flushed with dry N₂ and taken up in DCM (1.0 mL). The solution was stirred at ambient temperature and treated with HCl (4 N in dioxane, 0.31 mL). The reaction was stirred for about 30 minutes at which point it was briefly sonicated to break up the formed precipitate. After about an hour, the solution was diluted with diethyl ether and the precipitated solids collected via filtration and washed with additional ether. The crude product was dissolved in DMSO and purified by preparatory HPLC to afford the title compound. ES/MS (m/z) 713.2 (M+H)+. 1H NMR (400 MHz, DMSO-d6) δ 9.05 (s, 1H), 8.86 (d, J=4.1 Hz, 1H), 8.76 (d, J=8.0 Hz, 1H), 8.73-8.67 (m, 1H), 8.60 (dd, J=5.0, 1.4 Hz, 1H), 7.92 (t, J=5.1 Hz, 1H), 7.77 (d, J=7.5 Hz, 1H), 7.73-7.66 (m, 2H), 6.49-6.37 (m, 3H), 4.81-4.70 (m, 1H), 4.66-4.52 (m, 1H), 4.17-4.04 (m, 2H), 3.85-3.68 (m, 3H), 3.66 (d, J=3.0 Hz, 3H), 3.45 (dd, J=14.6, 10.5 Hz, 1H), 1.98 (s, 3H).

### Comparative Example 1 (CE-1)

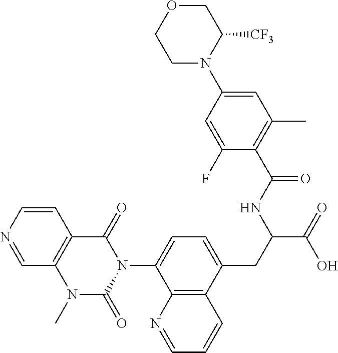

2-(2-fluoro-6-methyl-4-(R)-3-(trifluoromethyl)moropholino)benzamide)-3-(8-(1-methyl-2,4-dioxo-1,4-dihydropyrido[3,4-d]pyrimidin-3(2H)-yl)quinolin-5-yl)propanoic acid (CE-1): was synthesized according to know methods, such as, those disclosed in U.S. Pat. No. 11,116,760, the contents of which are incorporated herein by reference in their entirety.

### Comparative Example 2 (CE-2)

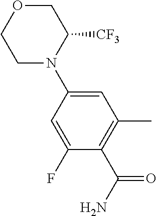 [SMILES: Cc1cc(N2CCOC[C@@H]2C(F)(F)F)cc(F)c1C(N)=O]

Step1: synthesis of 2-fluoro-N,N-bis[(4-methoxyphenyl)methyl]-6-methyl-4-[(3R)-3-(trifluoromethyl)morpholin-4-yl]benzamide: N-[(Dimethylamino)-1H-1,2,3-triazolo-[4,5-b]pyridin-1-ylmethylene]-N-methylmethanaminium hexafluorophosphate N-oxide (HATU, 1.85 g, 4.39 mmol), triethylamine (1.36 mL, 9.8 mmol), and 1-(4-methoxyphenyl)-N-[(4-methoxyphenyl)methyl]methanamine (921 mg, 3.58 mmol) was added to a flask containing 2-fluoro-6-methyl-4-[(3R)-3-(trifluoromethyl)morpholin-4-yl]benzoic acid (lg, 3.25 mmol) in dichloromethane (5 L). The resulting mixture was allowed to stir for about one hour. Next, the mixture was directly loaded onto silica gel and chromatographed using eluting with a gradient of hexanes and ethyl acetate to yield 1.4 g (2.56 mmol, 79%) of the titled compound.

Step 2: synthesis of 2-fluoro-6-methyl-4-[(3R)-3-(trifluoromethyl)morpholin-4-yl]benzamide (CE-2): To a tube was added 2-fluoro-N,N-bis[(4-methoxyphenyl)methyl]-6-methyl-4-[(3R)-3-(trifluoromethyl)morpholin-4-yl]benzamide (1.4 g, 2.56 mmol), and trifluoroacetic acid (25 mL). The tube was sealed and heated at 65 °C for about 66 hours. The volatile components were removed on a rotary evaporator and the residue as taken up in dichloromethane and chromatographed on silica gel eluting with a gradient of ethyl acetate and hexanes to provide CE-2 (626 mg, 2 mmol, 80% [M+H]=306.99)

### Receptor Occupancy (RO) Assay

The pharmacodynamics (PD) of compounds of Formula's III and IV were evaluated in healthy volunteers by measuring the receptor occupancy (RO) on α4β7 receptors in human whole blood CD4+ T_{memory} and CD8+ T_{memory} cells.

A whole blood RO assay that measures free α4β7 receptors on β7+CD4+CD45RO+ memory T cells and β7+CD8+CD45RO+ memory T-cells in comparison with a compound having high α4β7 receptor occupancy (CE-1), and a compound having low α4β7 receptor occupancy (CE-2) was utilized. Fresh whole blood B7+CD4+CD45RO+ memory T-cells and B7+CD8+CD45RO+ memory T-cells were identified using a staining panel comprised of fluorescently labelled anti-CD3, -CD4, -CD8, -CD45RO, and -B7 antibodies (Biolegend) (Table 34). Biotinylated human MAdCAM-1 protein, the natural ligand for α4β7, is mixed with streptavidin PE dextramer (Immudex) at a ratio of 10.4:1 (MDX) and added to the whole blood in the presence of the divalent cation manganese (as MnCl₂). MDX and the metabolite compete for binding to the α4β7 integrin receptor. Sample data was acquired immediately on a BD LSRFortessa X-20 Cell Analyzer flow cytometer using a α4β7 RO cell acquisition template and application settings. The gating strategy is as follows: lymphocytes are gated based on size (FSC-A) and complexity (SSC-A). Doublets are removed using FSC-A/FSC-H. CD3+ lymphocytes are further differentiated into CD4+ and CD8+ T cells. CD4+CD45RO+ positive or CD8+CD45RO+ T cells are gated on P7 integrin and MDX positivity. The fluorescent intensities of PE-MDX β7+CD4+CD45RO+ memory T cells or PE-MDX β7+CD8+CD45RO+ memory T cells in whole blood are used to calculate the % RO. The lower level of quantification (LLOQ) of the assay was estimated to be ~40% for a single measurement; similarly, the α4β7 RO assay was fit-for purpose validated to precisely quantify % RO up to 95% RO. Actual measured values <40% RO and ≥95% RO are shown but represent best estimates.

TABLE 34
Cell surface markers and antibodies α4β7 RO assay

| Fluorochrome | AF488 | BV510 | PerCP Cy5.5 | BV421 | APC | PE |
|---|---|---|---|---|---|---|
| Marker | CD3 | CD4 | CD8 | CD45RO | β7 | Biotinylated |
| | | | | | | MAdCAM-1 + SA-PE |

### Detailed Procedure

MDX solution: To a 1.5 mL microtube add 6.6 μL of 22.2 μM human biotinylated MAdCAM-1 and 87.84 μL of 160 nM SA-dextramer-PE and mix well. The solution is incubated for 30 minutes at room temperature in the dark. Upon completion, the solution is stored at 4 °C in the dark. When ready to use, add 1822.04 μl of cell staining buffer and mix, bringing total volume to 1916.48 μl.

Linage Antibody Master Mixture: To a 50 mL conical tube containing 23.68 ml of cell staining buffer add 384 μL anti-CD45RO BV421, 384 μL anti-CD3 A488, 384 μL anti-CD8 PerCPCy5.5, 384 μL anti-CD4 BV510, 384 μL anti-integrin P7 APC, mix on a vortex mixer.

To a 96 well U-bottom plate add 100 μL of blood per well. Next, equilibrate the blood in a 37 °C cell culture incubator (5% CO₂) for 30 minutes. Once incubation is completed remove from incubator and add CE-1, CE-2, Formula III and Formula IV compounds (final dosage range of 0.0007 nM to 10 μM) using a Tecan or comparable dispenser, mix well and incubate for 1 hr. in 37 °C incubator. Upon completion, add 5 μL of MDX solution followed by 5 μL of 44 mM MnCL₂ per well, mix well and incubate plate at room temperature for 30 minutes in the dark. Once the incubation was completed, the samples were transferred to a deep well 96 well plate containing 1.7 mL/well of 1×FACS lysing solution and mix well and then incubate for 10 minutes in the dark. Upon completion of the incubation, the plate was then centrifuged at 500 g for 5 minutes at room temperature and washed one time with 1.7 ml cell staining buffer. Next, the supernatants were discarded, and the cell pellets were resuspended in 200 μL of cell staining buffer, then transfer to samples to 96 well V-bottom plate. The 96 well V-bottom plate was then centrifuged at 500 g for 5 minutes at room temperature. The supernatants were discarded, and the cell pellets were resuspended in 100 μL of the lineage antibody master mixture. The Samples were then incubated at room temperature for 30 minutes in the dark. Next, 100 μL of cell staining buffer was added to each well. The 96 well V-bottom plate was centrifuged at 500 g for 5 minutes at room temperature. The supernatants were discarded, and the cell pellets were resuspended in 200 μL of cell staining buffer. The plates were then centrifuged at 500 g for 5 min. at room temperature. The supernatant was discarded, and the cell pellets were resuspended in 150 μL of cell staining buffer. The samples were then analyzed in the 96 well V-bottom plate on a LSRFortessa X-20 Cell Analyzer flow cytometer.

### Results

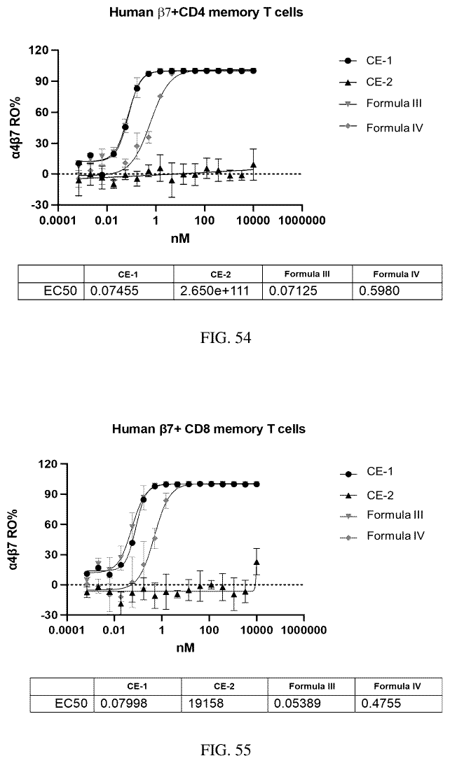

α4β7% RO were compared following the administration of dose titration of from about 0.0007 nM to about 10 μM for both Human β7+CD4 T_{memory} cells and Human β7+CD8 T_{memory} cells, FIGS. 54 and 55. Formula III has similar binding potency to CE-1 with an EC₅₀ of about 0.071 nM compared to 0.075 nM with CE-1 in β7+CD4 T_{memory} cells. Formula IV has about an 8-fold lower potency, 0.6 nM, in β7+CD4 T_{memory} cells when compared to CE-1. In β7+CD8 T_{memory} cells Formula III again has a slightly higher binding potency than CE-1 with an EC₅₀ of about 0.054 nM compared to 0.079 nM for CE-1. Formula IV has about a 6-fold lower potency, 0.48 nM, in β7+CD8 T_{memory} cells when compared to CE-1. The assay indicates that both Formula III and Formula IV are both modulators of α4β7 with Formula III being more potency than Formula IV.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A solid form which is:
(1) a solid form of a compound of Formula I: 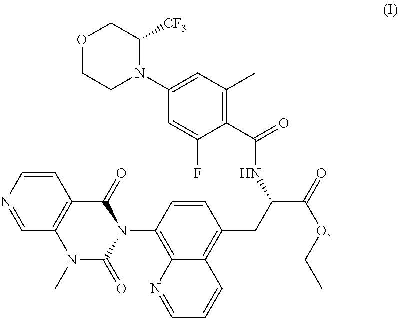
wherein the solid form is:
(a) Formula I freebase Form I, which is characterized by an X-Ray diffraction pattern having 2θ-reflections at 9.5, 21.0, and 25.7 degrees 2θ, plus or minus 0.2 degrees 2θ; an X-Ray diffraction pattern having 2θ-reflections at 10.5, 16.7, and 22.0 degrees 2θ, plus or minus 0.2 degrees 2θ; an X-Ray diffraction pattern having 2θ-reflections at 11.1, 13.8, and 23.3 degrees 2θ, plus or minus 0.2 degrees 2θ; an X-Ray diffraction substantially as shown in FIG. 1; a differential scanning calorimetry thermogram comprising an endothermic transition with an onset at about 177 °C; a differential scanning calorimetry thermogram substantially as shown in FIG. 2; and/or a thermogravimetric analysis substantially as shown in FIG. 3;
(b) Formula I freebase acetophenone solvate, which is characterized by an X-Ray diffraction pattern having 2θ-reflections at 8.3, 11.5, and 15.3 degrees 2θ, plus or minus 0.2 degrees 2θ; an X-Ray diffraction pattern having 2θ-reflections at 11.9, 16.4, and 18.3 degrees 2θ, plus or minus 0.2 degrees 2θ; an X-Ray diffraction pattern having 2θ-reflections at 12.0, 16.6, and 21.0 degrees 2θ, plus or minus 0.2 degrees 2θ; an X-Ray diffraction substantially as shown in FIG. 4; a differential scanning calorimetry thermogram with an endothermic transition with an onset at about 164 °C; a differential scanning calorimetry thermogram substantially as shown in FIG. 5; and/or a thermogravimetric analysis substantially as shown in FIG. 6; or
(c) Formula I freebase anisole solvate, which is characterized by an X-Ray diffraction pattern having 2θ-reflections at 11.5, 15.4, and 18.3 degrees 2θ, plus or minus 0.2 degrees 2θ; an X-Ray diffraction pattern having 2θ-reflections at 11.9, 16.8, and 21.4 degrees 2θ, plus or minus 0.2 degrees 2θ; an X-Ray diffraction pattern having 2θ-reflections at 1.6, 19.2, and 24.6 degrees 2θ, plus or minus 0.2 degrees 2θ; an X-Ray diffraction substantially as shown in FIG. 7; a differential scanning calorimetry thermogram with an endothermic transition with an onset at about 157 °C; a differential scanning calorimetry thermogram substantially as shown in FIG. 8; and/or a thermogravimetric analysis substantially as shown in FIG. 9;
(2) a solid form of a compound of the following formula: 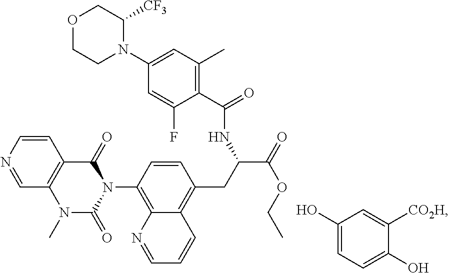
wherein the solid form is Formula I gentisate Form I, which is characterized by an X-Ray diffraction pattern having 2θ-reflections at 5.9, 16.4, and 21.8 degrees 2θ, plus or minus 0.2 degrees 2θ; an X-Ray diffraction pattern having 2θ-reflections at 4.8, 13.8, and 17.1 degrees 2θ, plus or minus 0.2 degrees 2θ; an X-Ray diffraction pattern having 2θ-reflections at 9.6, 11.7, and 25.0 degrees 2θ, plus or minus 0.2 degrees 2θ; an X-Ray diffraction substantially as shown in FIG. 10; a differential scanning calorimetry thermogram with an endothermic transition with an onset at about 147 °C; a differential scanning calorimetry thermogram substantially as shown in FIG. 11; and/or a thermogravimetric analysis substantially as shown in FIG. 12;
(3) a solid form of a compound of the following formula: 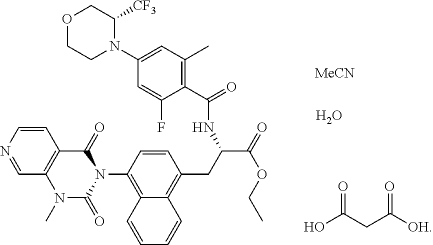
wherein the solid form is Formula I mono-malonate monohydrate acetonitrile solvate, which is characterized by an X-Ray diffraction pattern having 2θ-reflections at 7.5, 13.8, and 16.0 degrees 2θ, plus or minus 0.2 degrees 2θ; an X-Ray diffraction pattern having 2θ-reflections at 7.8, 12.1, and 19.2 degrees 2θ, plus or minus 0.2 degrees 2θ; an X-Ray diffraction pattern having 2θ-reflections at 11.5, 17.5, and 21.1 degrees 2θ, plus or minus 0.2 degrees 2θ; an X-Ray diffraction substantially as shown in FIG. 13; a differential scanning calorimetry thermogram with an endothermic transition with an onset at about 80 °C, and a second endothermic transition with an onset at about 126 °C; a differential scanning calorimetry thermogram substantially as shown in FIG. 14; and/or a thermogravimetric analysis substantially as shown in FIG. 15;
(4) a solid form of a compound of the following formula: 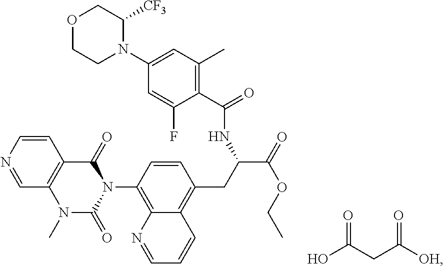
wherein the solid form is:
(a) Formula I mono-malonate Form I, which is characterized by an X-Ray diffraction pattern having 2θ-reflections at 8.1, 12.4, and 17.0 degrees 2θ, plus or minus 0.2 degrees 2θ; an X-Ray diffraction pattern having 2θ-reflections at 5.6, 16.7, and 20.0 degrees 2θ, plus or minus 0.2 degrees 2θ; an X-Ray diffraction pattern having 2θ-reflections at 8.9, 16.3, and 23.8 degrees 2θ, plus or minus 0.2 degrees 2θ; an X-Ray diffraction substantially as shown in FIG. 16; a differential scanning calorimetry thermogram with an endothermic transition with an onset at about 125 °C; a differential scanning calorimetry thermogram substantially as shown in FIG. 17; and/or a thermogravimetric analysis substantially as shown in FIG. 18; or
(b) Formula I mono-malonate Form II, which is characterized by an X-Ray diffraction pattern having 2θ-reflections at 12.5, 13.1, and 15.5 degrees 2θ, plus or minus 0.2 degrees 2θ; an X-Ray diffraction pattern having 2θ-reflections at 11.6, 14.6, and 16.2 degrees 2θ, plus or minus 0.2 degrees 2θ; an X-Ray diffraction pattern having 2θ-reflections at 7.9, 17.4, and 23.0 degrees 2θ, plus or minus 0.2 degrees 2θ; an X-Ray diffraction substantially as shown in FIG. 19; a differential scanning calorimetry thermogram with an endothermic transition with an onset at about 128 °C: a differential scanning calorimetry thermogram substantially as shown in FIG. 20; and/or a thermogravimetric analysis substantially as shown in FIG. 21;
(5) a solid form of a compound of the following formula: 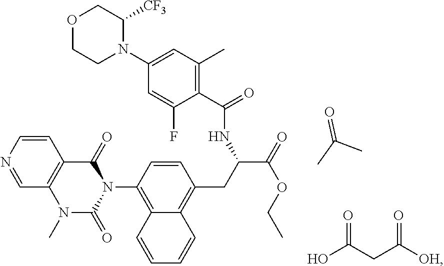
wherein the solid form is Formula I mono-malonate acetone solvate, which is characterized by an X-Ray diffraction pattern having 2θ-reflections at 7.5, 7.8, and 15.6 degrees 2θ, plus or minus 0.2 degrees 2θ; an X-Ray diffraction pattern having 2θ-reflections at 12.3, 13.9, and 16.0 degrees 2θ, plus or minus 0.2 degrees 2θ; an X-Ray diffraction pattern having 2θ-reflections at 17.7, 19.4, and 21.0 degrees 2θ, plus or minus 0.2 degrees 2θ; and/or an X-Ray diffraction substantially as shown in FIG. 22;
(6) a solid form of a compound of the following formula: 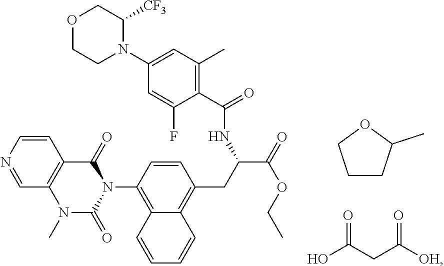
wherein the solid form is Formula I mono-malonate 2-methyltetrahydrofuran solvate, which is characterized by an X-Ray diffraction pattern having 2θ-reflections at 7.4, 12.0, and 13.6 degrees 2θ, plus or minus 0.2 degrees 2θ; an X-Ray diffraction pattern having 2θ-reflections at 7.7, 11.3, and 15.5 degrees 2θ, plus or minus 0.2 degrees 2θ; an X-Ray diffraction pattern having 2θ-reflections at 15.9, 18.3, and 20.8 degrees 2θ, plus or minus 0.2 degrees 2θ; and/or an X-Ray diffraction substantially as shown in FIG. 23;
(7) a solid form of a compound of the following formula: 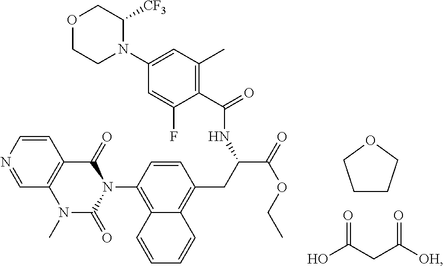
wherein the solid form is Formula I mono-malonate tetrahydrofuran solvate, which is characterized by an X-Ray diffraction pattern having 2θ-reflections at 7.9, 15.7, and 21.1 degrees 2θ, plus or minus 0.2 degrees 2θ; an X-Ray diffraction pattern having 2θ-reflections at 7.5, 13.9, and 19.4 degrees 2θ, plus or minus 0.2 degrees 2θ; an X-Ray diffraction pattern having 2θ-reflections at 13.7, 17.4, and 20.0 degrees 2θ, plus or minus 0.2 degrees 2θ; and/or an X-Ray diffraction substantially as shown in FIG. 24;
(8) a solid form of a compound of the following formula: 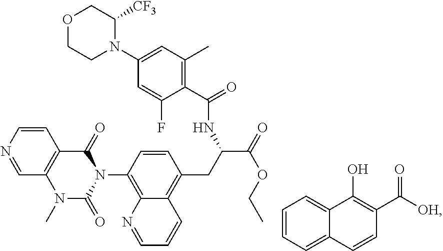
wherein the solid form is:
(a) Formula I xinafoate Form I, which is characterized by an X-Ray diffraction pattern having 2θ-reflections at 4.3, 6.7, and 15.5 degrees 2θ, plus or minus 0.2 degrees 2θ; an X-Ray diffraction pattern having 2θ-reflections at 8.9, 13.2, and 15.8 degrees 2θ, plus or minus 0.2 degrees 2θ; an X-Ray diffraction pattern having 2θ-reflections at 17.9, 20.9, and 24.8 degrees 2θ, plus or minus 0.2 degrees 2θ; and/or an X-Ray diffraction substantially as shown in FIG. 25; or
(b) Formula I xinafoate Form II, which is characterized by an X-Ray diffraction pattern having 2θ-reflections at 9.4, 15.3, and 18.9 degrees 2θ, plus or minus 0.2 degrees 2θ; an X-Ray diffraction pattern having 2θ-reflections at 4.6, 13.5, and 16.9 degrees 2θ, plus or minus 0.2 degrees 2θ; an X-Ray diffraction pattern having 2θ-reflections at 6.6, 13.2, and 21.2 degrees 2θ, plus or minus 0.2 degrees 2θ; an X-Ray diffraction substantially as shown in FIG. 26; a differential scanning calorimetry thermogram with an endothermic transition with an onset at about 45 °C, and a second endothermic transition with an onset at about 124 °C; a differential scanning calorimetry thermogram substantially as shown in FIG. 27; and/or a thermogravimetric analysis substantially as shown in FIG. 28;
(9) a solid form of a compound of the following formula: 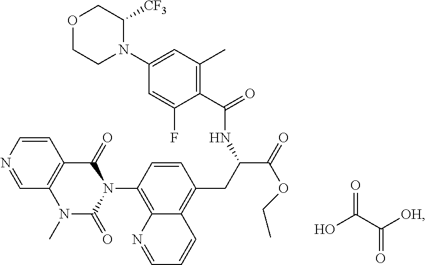
wherein the solid form is Formula I oxalate, which is characterized by an X-Ray diffraction pattern having 2θ-reflections at 4.4, 8.0, and 16.5 degrees 2θ, plus or minus 0.2 degrees 2θ; an X-Ray diffraction pattern having 2θ-reflections at 6.8, 8.4, and 10.2 degrees 2θ, plus or minus 0.2 degrees 2θ; an X-Ray diffraction pattern having 2θ-reflections at 6.5, 8.9, and 12.8 degrees 2θ, plus or minus 0.2 degrees 2θ; an X-Ray diffraction substantially as shown in FIG. 29; a differential scanning calorimetry thermogram with an endothermic transition with an onset at about 155 °C; a differential scanning calorimetry thermogram substantially as shown in FIG. 30; and/or a thermogravimetric analysis substantially as shown in FIG. 31;
(10) a solid form of a compound of the following formula: 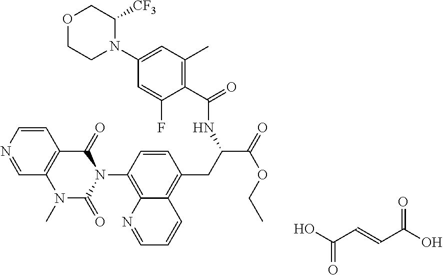
wherein the solid form is:
(a) Formula I fumarate Form I, which is characterized by an X-Ray diffraction pattern having 2θ-reflections at 5.7, 11.4, and 14.4 degrees 2θ, plus or minus 0.2 degrees 2θ; an X-Ray diffraction pattern having 2θ-reflections at 13.0, 21.4, and 26.2 degrees 2θ, plus or minus 0.2 degrees 2θ; an X-Ray diffraction pattern having 2θ-reflections at 15.3, 18.4, and 28.8 degrees 2θ, plus or minus 0.2 degrees 2θ; an X-Ray diffraction substantially as shown in FIG. 32; a differential scanning calorimetry thermogram with an endothermic transition with an onset at about 31 °C and a second endothermic transition with an onset at about 145 °C; a differential scanning calorimetry thermogram substantially as shown in FIG. 33; and/or a thermogravimetric analysis substantially as shown in FIG. 34;
(b) Formula I fumarate Form II, which is characterized by an X-Ray diffraction pattern having 2θ-reflections at 5.6, 6.1, and 16.5 degrees 2θ, plus or minus 0.2 degrees 2θ; an X-Ray diffraction pattern having 2θ-reflections at 13.6, 16.2, and 24.7 degrees 2θ, plus or minus 0.2 degrees 2θ; an X-Ray diffraction pattern having 2θ-reflections at 15.6, 18.4, and 25.2 degrees 2θ, plus or minus 0.2 degrees 2θ; an X-Ray diffraction substantially as shown in FIG. 35; a differential scanning calorimetry thermogram with a broad endothermic transition with an onset at about 25 °C and a second endothermic transition with an onset at about 162 °C; a differential scanning calorimetry thermogram substantially as shown in FIG. 36; and/or a thermogravimetric analysis substantially as shown in FIG. 37; or
(c) Formula I fumarate Form III, which is characterized by an X-Ray diffraction pattern having 2θ-reflections at 6.4, 10.9, and 15.5 degrees 2θ, plus or minus 0.2 degrees 2θ; an X-Ray diffraction pattern having 2θ-reflections at 5.5, 14.4, and 18.2 degrees 2θ, plus or minus 0.2 degrees 2θ; an X-Ray diffraction pattern having 2θ-reflections at 10.6, 16.6, and 22.0 degrees 2θ, plus or minus 0.2 degrees 2θ; and/or an X-Ray diffraction substantially as shown in FIG. 38;
(11) a solid form of a compound of the following formula: 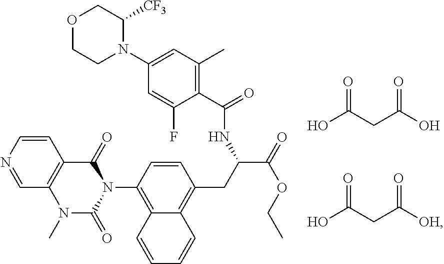
wherein the solid form is Formula I bis-malonate Form I, which is characterized by an X-Ray diffraction pattern having 2θ-reflections at 6.9, 13.5, and 20.4 degrees 2θ, plus or minus 0.2 degrees 2θ; an X-Ray diffraction pattern having 2θ-reflections at 11.6, 18.4, and 23.6 degrees 2θ, plus or minus 0.2 degrees 2θ; an X-Ray diffraction pattern having 2θ-reflections at 11.2, 12.9, and 16.2 degrees 2θ, plus or minus 0.2 degrees 2θ; an X-Ray diffraction substantially as shown in FIG. 39; a differential scanning calorimetry thermogram with an endothermic transition with an onset at about 114 °C; a differential scanning calorimetry thermogram substantially as shown in FIG. 40; and/or a thermogravimetric analysis substantially as shown in FIG. 41;
(12) a solid form of a compound of the following formula: 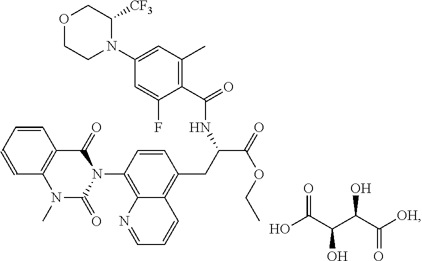
wherein the solid form is Formula I L-tartrate, which is characterized by an X-Ray diffraction pattern having 2θ-reflections at 4.7, 6.4, and 13.8 degrees 2θ, plus or minus 0.2 degrees 2θ; an X-Ray diffraction pattern having 2θ-reflections at 9.6, 13.2, and 18.8 degrees 2θ, plus or minus 0.2 degrees 2θ; an X-Ray diffraction pattern having 2θ-reflections at 15.6, 16.4, and 17.5 degrees 2θ, plus or minus 0.2 degrees 2θ; an X-Ray diffraction substantially as shown in FIG. 42; a differential scanning calorimetry thermogram with a broad endothermic transition followed by a second endothermic transition with an onset at about 156 °C; a differential scanning calorimetry thermogram substantially as shown in FIG. 43; and/or a thermogravimetric analysis substantially as shown in FIG. 44;
(13) a solid form of a compound of the following formula: 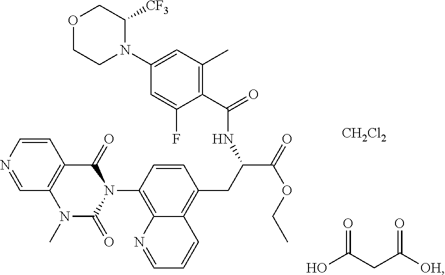
wherein the solid form is Formula I mono-malonate dichloromethane solvate, which is characterized by an X-Ray diffraction pattern having 2θ-reflections at 7.5, 13.8, and 15.7 degrees 2θ, plus or minus 0.2 degrees 2θ; an X-Ray diffraction pattern having 2θ-reflections at 7.8, 11.5, and 16.0 degrees 2θ, plus or minus 0.2 degrees 2θ; an X-Ray diffraction pattern having 2θ-reflections at 12.2, 17.5, and 21.0 degrees 2θ, plus or minus 0.2 degrees 2θ; and/or an X-Ray diffraction substantially as shown in FIG. 52; or
(14) a solid form of a compound of the following formula: 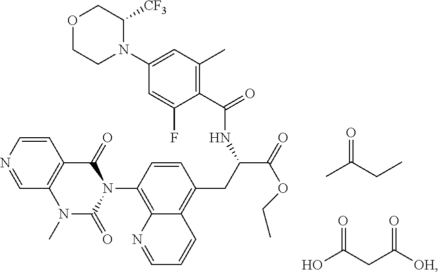
wherein the solid form is Formula I mono-malonate methyl ketone solvate, which is characterized by an X-Ray diffraction pattern having 2θ-reflections at 7.4, 13.6, and 16.0 degrees 2θ, plus or minus 0.2 degrees 2θ; an X-Ray diffraction pattern having 2θ-reflections at 7.9, 12.1, and 15.7 degrees 2θ, plus or minus 0.2 degrees 2θ; an X-Ray diffraction pattern having 2θ-reflections at 11.4, 17.4, and 19.4 degrees 2θ, plus or minus 0.2 degrees 2θ; and/or an X-Ray diffraction substantially as shown in FIG. 53.

2. The solid form of claim 1, wherein the solid form is Formula I freebase Form I, which is characterized by an X-Ray diffraction pattern having 2θ-reflections at 9.5, 21.0, and 25.7 degrees 2θ, plus or minus 0.2 degrees 2θ; an X-Ray diffraction pattern having 2θ-reflections at 10.5, 16.7, and 22.0 degrees 2θ, plus or minus 0.2 degrees 2θ; an X-Ray diffraction pattern having 2θ-reflections at 11.1, 13.8, and 23.3 degrees 2θ, plus or minus 0.2 degrees 2θ; an X-Ray diffraction substantially as shown in FIG. 1; a differential scanning calorimetry thermogram comprising an endothermic transition with an onset at about 177 °C; a differential scanning calorimetry thermogram substantially as shown in FIG. 2; and/or a thermogravimetric analysis substantially as shown in FIG. 3.

3-35. (canceled)

36. The solid form of claim 1, wherein the solid form is Formula I mono-malonate monohydrate acetonitrile solvate, which is characterized by an X-Ray diffraction pattern having 2θ-reflections at 7.5, 13.8, and 16.0 degrees 2θ, plus or minus 0.2 degrees 2θ; an X-Ray diffraction pattern having 2θ-reflections at 7.8, 12.1, and 19.2 degrees 2θ, plus or minus 0.2 degrees 2θ; an X-Ray diffraction pattern having 2θ-reflections at 11.5, 17.5, and 21.1 degrees 2θ, plus or minus 0.2 degrees 2θ; an X-Ray diffraction substantially as shown in FIG. 13; a differential scanning calorimetry thermogram with an endothermic transition with an onset at about 80 °C, and a second endothermic transition with an onset at about 126 °C; a differential scanning calorimetry thermogram substantially as shown in FIG. 14; and/or a thermogravimetric analysis substantially as shown in FIG. 15.

37.-44. (canceled)

45. The solid form of claim 1, wherein the solid form is Formula I mono-malonate Form I, which is characterized by an X-Ray diffraction pattern having 2θ-reflections at 8.1, 12.4, and 17.0 degrees 2θ, plus or minus 0.2 degrees 2θ; an X-Ray diffraction pattern having 2θ-reflections at 5.6, 16.7, and 20.0 degrees 2θ, plus or minus 0.2 degrees 2θ; an X-Ray diffraction pattern having 2θ-reflections at 8.9, 16.3, and 23.8 degrees 2θ, plus or minus 0.2 degrees 2θ; an X-Ray diffraction substantially as shown in FIG. 16; a differential scanning calorimetry thermogram with an endothermic transition with an onset at about 125 °C; a differential scanning calorimetry thermogram substantially as shown in FIG. 17; and/or a thermogravimetric analysis substantially as shown in FIG. 18.

46.-141. (canceled)

142. A solid form of a compound of Formula II: 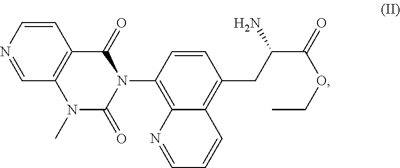
wherein the solid form is:
(1) Formula II Form I, which is characterized by an X-Ray diffraction pattern having 2θ-reflections at 7.2, 9.9, and 12.5 degrees 2θ, plus or minus 0.2 degrees 2θ; an X-Ray diffraction pattern having 2θ-reflections at 6.8, 8.0, and 15.3 degrees 2θ, plus or minus 0.2 degrees 2θ; an X-Ray diffraction pattern having 2θ-reflections at 14.7, 17.6, and 19.8 degrees 2θ, plus or minus 0.2 degrees 2θ; and/or an X-Ray diffraction substantially as shown in FIG. 45;
(2) Formula II Form II, which is characterized by an X-Ray diffraction pattern having 2θ-reflections at 8.6, 14.0, and 16.7 degrees 2θ, plus or minus 0.2 degrees 2θ; an X-Ray diffraction pattern having 2θ-reflections at 12.6, 14.8, and 17.8 degrees 2θ, plus or minus 0.2 degrees 2θ; an X-Ray diffraction pattern having 2θ-reflections at 16.4, 18.4, and 19.7 degrees 2θ, plus or minus 0.2 degrees 2θ; an X-Ray diffraction substantially as shown in FIG. 46; a differential scanning calorimetry thermogram with an endothermic transition with an onset at about 104 °C followed by a second endothermic transition with an onset at about 152 °C; a differential scanning calorimetry thermogram substantially as shown in FIG. 47; and/or a thermogravimetric analysis substantially as shown in FIG. 48;
(3) Formula II Form III, which is characterized by an X-Ray diffraction pattern having 2θ-reflections at 7.3, 10.0, and 16.2 degrees 2θ, plus or minus 0.2 degrees 2θ; an X-Ray diffraction pattern having 2θ-reflections at 8.1, 12.5, and 17.9 degrees 2θ, plus or minus 0.2 degrees 2θ; an X-Ray diffraction pattern having 2θ-reflections at 6.7, 15.2, and 25.9 degrees 2θ, plus or minus 0.2 degrees 2θ; and/or an X-Ray diffraction substantially as shown in FIG. 49;
(4) Formula II Form IV, which is characterized by an X-Ray diffraction pattern having 2θ-reflections at 4.7, 11.0, and 12.5 degrees 2θ, plus or minus 0.2 degrees 2θ; an X-Ray diffraction pattern having 2θ-reflections at 11.3, 11.5, and 16.4 degrees 2θ, plus or minus 0.2 degrees 2θ; an X-Ray diffraction pattern having 2θ-reflections at 13.9, 14.1, and 17.8 degrees 2θ, plus or minus 0.2 degrees 2θ; an X-Ray diffraction substantially as shown in FIG. 50; and/or a differential scanning calorimetry thermogram substantially as shown in FIG. 56;
(5) Formula II Form V, which is characterized by an X-Ray diffraction pattern having 2θ-reflections at 8.6, 12.4, and 15.0 degrees 2θ, plus or minus 0.2 degrees 2θ; an X-Ray diffraction pattern having 2θ-reflections at 14.3, 16.7, and 17.9 degrees 2θ, plus or minus 0.2 degrees 2θ; an X-Ray diffraction pattern having 2θ-reflections at 17.2, 18.5, and 19.8 degrees 2θ, plus or minus 0.2 degrees 2θ; and/or an X-Ray diffraction substantially as shown in FIG. 51;
(6) Formula II Form VI, which is characterized by an X-Ray diffraction pattern having 2θ-reflections at 5.8, 12.8, and 7.8 degrees 2θ, plus or minus 0.2 degrees 2θ; an X-Ray diffraction pattern having 2θ-reflections at 12.0, 18.4, and 24.2 degrees 2θ, plus or minus 0.2 degrees 2θ; an X-Ray diffraction pattern having 2θ-reflections at 11.4, 15.5, and 19.0 degrees 2θ, plus or minus 0.2 degrees 2θ; an X-Ray diffraction substantially as shown in FIG. 57; and/or a differential scanning calorimetry thermogram substantially as shown in FIG. 58;
(7) Formula II Form VII, which is characterized by an X-Ray diffraction pattern having 2θ-reflections at 8.6, 10.8, and 17.2 degrees 2θ, plus or minus 0.2 degrees 2θ; an X-Ray diffraction pattern having 2θ-reflections at 8.0, 12.0, and 15.7 degrees 2θ, plus or minus 0.2 degrees 2θ; an X-Ray diffraction pattern having 2θ-reflections at 15.2, 20.2, and 24.2 degrees 2θ, plus or minus 0.2 degrees 2θ; and/or an X-Ray diffraction substantially as shown in FIG. 59;
(8) Formula II Form VIII, which is characterized by an X-Ray diffraction pattern having 2θ-reflections at 7.4, 9.9, and 16.5 degrees 2θ, plus or minus 0.2 degrees 2θ; an X-Ray diffraction pattern having 2θ-reflections at 7.0, 10.4, and 22.5 degrees 2θ, plus or minus 0.2 degrees 2θ; an X-Ray diffraction pattern having 2θ-reflections at 9.1, 15.2, and 24.4 degrees 2θ, plus or minus 0.2 degrees 2θ; and/or an X-Ray diffraction substantially as shown in FIG. 60;
(9) Formula II Form IX, which is characterized by an X-Ray diffraction pattern having 2θ-reflections at 7.2, 9.8, and 13.4 degrees 2θ, plus or minus 0.2 degrees 2θ; an X-Ray diffraction pattern having 2θ-reflections at 7.9, 15.2, and 17.9 degrees 2θ, plus or minus 0.2 degrees 2θ; an X-Ray diffraction pattern having 2θ-reflections at 6.6, 12.3, and 16.3 degrees 2θ, plus or minus 0.2 degrees 2θ; and/or an X-Ray diffraction substantially as shown in FIG. 61; or
(10) Formula II Form X, which is characterized by an X-Ray diffraction pattern having 2θ-reflections at 7.2, 9.8, and 12.4 degrees 2θ, plus or minus 0.2 degrees 2θ; an X-Ray diffraction pattern having 2θ-reflections at 7.9, 17.6, and 20.8 degrees 2θ, plus or minus 0.2 degrees 2θ; an X-Ray diffraction pattern having 2θ-reflections at 6.7, 18.2, and 26.8 degrees 2θ, plus or minus 0.2 degrees 2θ; and/or an X-Ray diffraction substantially as shown in FIG. 62.

143-147. (canceled)

148. The solid form of claim 142, wherein the solid form is Formula II Form II, which is characterized by an X-Ray diffraction pattern having 2θ-reflections at 8.6, 14.0, and 16.7 degrees 2θ, plus or minus 0.2 degrees 2θ; an X-Ray diffraction pattern having 2θ-reflections at 12.6, 14.8, and 17.8 degrees 2θ, plus or minus 0.2 degrees 2θ; an X-Ray diffraction pattern having 2θ-reflections at 16.4, 18.4, and 19.7 degrees 2θ, plus or minus 0.2 degrees 2θ; an X-Ray diffraction substantially as shown in FIG. 46; a differential scanning calorimetry thermogram with an endothermic transition with an onset at about 104 °C followed by a second endothermic transition with an onset at about 152 °C; a differential scanning calorimetry thermogram substantially as shown in FIG. 47; and/or a thermogravimetric analysis substantially as shown in FIG. 48.

149.-171. (canceled)

172. A compound, or pharmaceutically acceptable salt thereof which is

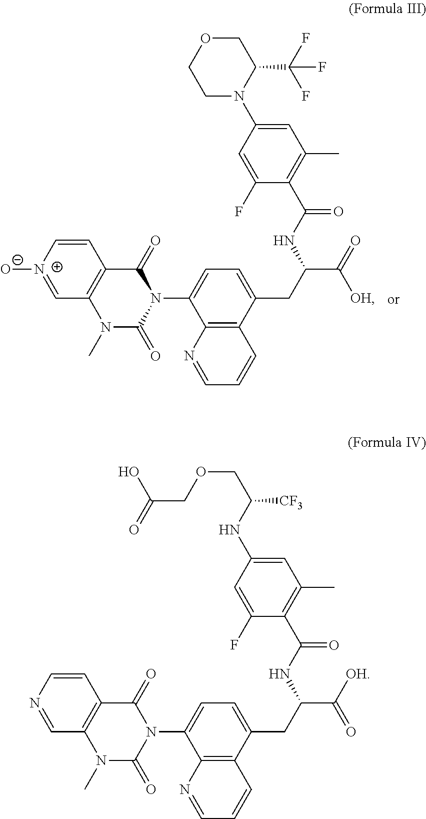

173. A pharmaceutical composition comprising a compound of claim 172 or a pharmaceutically acceptable salt thereof, and a pharmaceutically acceptable excipient.

174. The pharmaceutical composition of claim 173, further comprising one or more additional therapeutic agents.

175. (canceled)

176. A method for treating a disease or condition associated with α4β7 integrin comprising administrating to a subject an effective amount of the compound of claim 172.

177. The method of claim 176, wherein the disease or condition is Inflammatory bowel disease.

178. The method of claim 177, wherein the Inflammatory bowel disease is Ulcerative colitis.

179. The method of claim 177, wherein the Inflammatory bowel disease is Crohn's disease.

180. The method of claim 176, further comprising administering an additional therapeutic compound.

181.-218. (canceled)
